(19) United States
(12) Patent Application Publication
(10) Pub. No.: US 2008/0118576 A1
(43) Pub. Date: May 22, 2008

(54) PREDICTION OF AN AGENT'S OR AGENTS' ACTIVITY ACROSS DIFFERENT CELLS AND TISSUE TYPES

(76) Inventors: Dan Theodorescu, Charlottesville, VA (US); Jae Kyun Lee, Charlottesville, VA (US)

Correspondence Address: FELDMANGALE, P.A., ONE BISCAYNE TOWER , 30TH FLOOR , 2 SOUTH, BISCAYNE BOULEVARD, MIAMI, FL, 33131-4332, US

(21) Appl. No.: 11/846,340

(22) Filed: Aug. 28, 2007

Related U.S. Application Data
(60) Provisional application No. 60/840,644, filed on Aug. 28, 2006.
(60) Provisional application No. 60/840,834, filed on Aug. 28, 2006.

(51) Int. Cl. C40B 30/02; A61K 31/7072; A61K 33/26; A61K 31/337; A61P 35/00

(52) U.S. Cl. 424/649; 506/8; 514/449; 514/49

(57) ABSTRACT

The present invention relates to a novel algorithm that uses molecular profile signatures to extrapolate the physiological processes of one type of cell set (e.g., cell line, tissue, normal or diseased) to predict the activity of an agent or agents against another type of cell set that has never been exposed to the agent in question (drug efficacy prediction). The novel algorithm also allows one to predict the therapeutic response of a patient to a therapeutic regimen even though the patient (or patients) may have never been exposed to that agent before, thereby allowing for selecting a therapeutic agent or combination of agents that would best suit the patient (i.e., personalized medicine). The present invention also relates to methods of using the agents identified by the novel algorithm to treat a variety of diseases, including cancer.

### CROSS-REFERENCE TO RELATED APPLICATIONS

The present application claims priority benefit under 35 U.S.C. § 119(e) of U.S. Provisional Patent Application Ser. No. 60/840,644 filed Aug. 28, 2006 and U.S. Provisional Patent Application Ser. No. 60/840,834 filed Nov. 22, 2006. The disclosures of these applications are incorporated herein by reference.

### FIELD OF THE INVENTION

The present invention relates to a novel algorithm that uses molecular profile signatures to extrapolate the physiological processes of one type of cell set (e.g., cell line, tissue, normal or diseased) to predict the activity of an agent or agents against another type of cell set that has never been exposed to the agent in question (drug efficacy prediction). The novel algorithm also allows one to predict the therapeutic response of a patient(s) to a therapeutic regimen even though the patient(s) may have never been exposed to that agent before, thereby allowing for selecting a therapeutic agent or combination of agents that would best suit the patient(s) (i.e., personalized medicine). The present invention also relates to methods of using the agents identified by the novel algorithm to treat a variety of diseases, including cancer.

### BACKGROUND OF THE INVENTION

Tumors have traditionally been classified by descriptive characteristics such as organ of origin, histology, aggressiveness, and extent of spread. That empirical rubric is being challenged, however, as molecular-level classifications, made possible by microarrays and other high-throughput profiling technologies, become increasingly common and persuasive. The reductionist program would suggest that, eventually, all differences among traditional tumor types will be reduced to statements about molecules in the tumors and about the interactions among those molecules. It might then be possible to study physiological processes in one type of cancer and extrapolate the results to predict another type through commonalities in their molecular constitutions. This concept forms the basis for the claimed invention.

The NCI-60 cell line screen, which has been used by the Developmental Therapeutics Program (DTP) of the U.S. National Cancer Institute (NCI) to screen >100,000 chemically defined compounds plus a large number of natural product extracts for anticancer activity since 1990. The NCI-60 panel comprises 60 diverse human cancers, including leukemias, melanomas, and cancers of renal, ovarian, lung, colon, breast, prostate, and central nervous system origin. The NCI-60 have been comprehensively profiled at the DNA, RNA, protein, and functional levels, and the resulting information on molecular characteristics and their relationship to patterns of drug activity have proven fruitful for studies of drug mechanisms of action, resistance, and modulation.

Unfortunately, it was not feasible to include all important tumor types in the NCI-60. For example, there are no lymphomas, sarcomas, head and neck tumors, squamous cell carcinomas, small cell lung cancers, pancreatic cancers, or urothelial bladder cancers. Even if cancer cells of the additional histological types were added to the panel now, all compounds screened in the past 16 years would have to be tested again against the updated panel to gain the full predictive power of the database for the legacy compounds. Thus, it would be highly beneficial to discover a method of evaluating the activity of compounds in a computational, rather than experimental model in order to gather information on the drug sensitivity of these other tumors. A solution to this problem is provided by the claimed invention.

We are awash in novel anticancer agents. With a few notable exceptions, however, clinical successes have not followed proportionately with these discoveries. A fundamental reason for this problem is the lack of good predictive ability of early in vitro or xenograft based testing of new agents or combinations thereof to subsequent clinical responses in patients. The choice of therapy for metastatic cancer is thus largely empiric because of a lack of chemosensitivity prediction for available combination chemotherapeutic regimens. It is, therefore, highly desirable to discover methods of predicting the activity of agents in a manner that is predictive of both in vitro or xenograft activity and in vivo (human patient) activity. In addition to cancer, it is also desirable to discover methods of predicting the activity of agents against other disease targets (e.g., diabetes) without having to experimentally test each agent.

Most patients with epithelial cancers requiring systemic treatment undergo combination chemotherapy. However, a major challenge in these patients has been the prediction of chemotherapeutic efficacy of combination therapy. There are several reasons for this: First, it is difficult to select the most effective combination chemotherapy for each cancer patient when thousands of anticancer agents are only tested individually on cancer cells. Their effectiveness is not tested in combination on cancer cells due to the enormous undertaking this would pose. For example, if there are 10 candidate single agents for combination chemotherapy, we would have 45 doublet combinations, 120 triplets, and 210 quadruple combinations. Second, very few of these combinations are eventually tested in cancer patients. Third, there is the lack of good predictive ability of single-agent chemosensitivity in patients from in vitro or xenograft data. Fourth, there is the lack of good predictive ability of combination-agent chemosensitivity in patients from in vitro or xenograft data. It is, therefore, highly desirable to discover methods of predicting the activity of combinations of agents in a patient without having to experimentally test the activity of each combination in the patient. In addition to cancer, it is also desirable to discover methods of predicting the activity of combinations agents in a patent against other disease targets (e.g., diabetes) without having to experimentally test the activity of each combination in the patient.

### SUMMARY OF THE INVENTION

The present invention provides novel methods for predicting the activity of at least one agent or combination of agents on cell lines or animal tumors, tissues, or organs either syngeneic or xenograft without the cell lines or animal tumors, tissues, or organs either syngeneic or xenograft ever having been exposed to the agent—the predicting being based on the sensitivity of other cell lines or animal tumors, tissues, or organs either syngeneic or xenograft to the agent.

The present invention also provides novel methods of predicting the therapeutic effectiveness of an agent or combination of agents in a human patient without that patient's tumor/organ/tissue ever having been exposed to the agent—the predicting being based on the sensitivity of other human patient/patient's tumor/organ/tissue to said agent. For example, one benefit of the present invention is the ability to predict a patient's response to an agent without having testing that agent on that patient or even a test set of patients.

The present invention also provides novel methods of predicting which cell lines or animal tumors, tissues, or organs either syngeneic or xenograft or human tumors that are sensitive to a specific therapeutic agents-thereby allowing for personalized therapy.

The present invention also provides a set of genes, the expression of which is important for the prediction of treatment responses for any cancer (e.g., cancers of the bladder and breast) to any agent with activity in cell lines, animal tumors, tissues, or organs either syngeneic or xenograft or human tumors.

The present invention also provides a set of agents that have been found through use of the present invention to be effective in several human cancers including bladder, breast, prostate, pancreatic, and melanoma.

The present invention further provides methods of treating diseases with the agent(s) identified herein.

These and other aspects of the present invention were discovered through the creation of the algorithm described herein.

### BRIEF DESCRIPTION OF THE DRAWINGS

FIG. 1: Application of the gene co-expression extrapolation signature (COXEN) to the BLA-40 bladder cell lines: (A) Summary schematic diagram for chemosensitivity prediction model development and model validation. (B) Direct comparison between the standardized MiPP prediction scores and the standardized log(GI50) values on the BLA-40 for cisplatin. The sensitive (and resistant cell lines) are ordered based on their log(GI50) values (x-axis), which were obtained from in vitro chemosensitivity experiments. The standardized predicted MIPP scores are also depicted next to the standardized log(GI50) values of corresponding cell lines. The standardized scores were obtained by subtracting the overall mean divided by the standard deviation of the MiPP scores and log(GI50) values on the BLA-40. Statistical significance was determined using the Spearman correlation coefficient with p-value=0.016. (C) Direct comparison between the standardized MiPP prediction scores and the standardized log(GI50) values on the BLA-40 for paclitaxel. Statistical significance was determined using the Spearman correlation coefficient with p-value=0.006. (D) Receiver-operator characteristic (ROC) analysis. ROC curves were drawn for (1) the full COXEN algorithm and those obtained by leaving out either (2) the drug chemosensitivity signature step (Step 3, p-value=0.0053) or (3) the co-occurrence step (Step 5, p-value=0.0059). The Wilcoxon rank-sum tests were performed to obtain the statistical significance between different ROC curves. The comparison test between (2) and (3) ROC curves was insignificant (p-value=0.792).

FIG. 2: (A) Schematic illustration of co-expression extrapolation. In this artificial five-probe example, Probes 1 and 3 in Cell Set 1 (e.g., the NCI-60) show essentially the same patterns of co-expression correlation with other probes as do Probes 1 and 3 in Cell Set 2 (e.g., the BLA-40). Probes 2, 4, and 5 show different patterns of co-expression correlation in the two Cell Sets. Therefore, Probes 1 and 3 (but not 2, 4, and 5) might be selected by the “co-expression extrapolation” algorithm (Step 5) for inclusion in the prediction signature for Step 6. Note: The co-expression correlations here are those calculated across cell types for a given pair of probes. (Step 5). (B-E) Co-clustering Cluster Image Maps (CIMs) or heatmaps for chemosensitivity genes and for COXEN signature genes: (B) Co-clustering CIM between the NCI-60 and the BLA-40 cell lines using first 50 genes of the entire differentially expressed chemosensitivity probe sets of cisplatin. The red and green colors of the heatmap represent high and low expressions, respectively while intermediate expression is black. Bright red and blue bar (Upper panel) indicates sensitive cells and resistant cells of the NCI-60 and BLA-40 as defined in FIGS. 10A and C. Bright yellow and cyan (lower panel) indicate the NCI-60 and the BLA-40 cell lines. Most cell lines clustered based on their origins-NCI-60 and BLA-40 and the sensitive (or resistant) cell lines are not intermixed between the two cell line panels. (C) Co-clustering CIM between the NCI-60 and the BLA-40 cell lines of the final 18 COXEN genes for cisplatin (Supplemental Table S1). The sensitive (and resistant) of the NCI-60 and the BLA-40 cell lines were closely clustered, despite the differences in their tissue origins. (D) Co-clustering CIM between the NCI-60 and the BLA-40 cell lines using first 50 probes of the entire differentially expressed chemosensitivity probe sets of paclitaxel. The red and green colors of the heatmap represent high and low expressions, respectively while intermediate expression is black. Bright red and blue bar (upper panel) indicates sensitive cells and resistant cells of the NCI-60 and BLA-40 as defined in FIGS. 10B and D. Bright yellow and cyan (lower panel) indicate the NCI-60 and the BLA-40 cell lines. Most cell lines clustered based on their origins-NCI-60 and BLA-40 and the sensitive (or resistant) cell lines are not intermixed between the two cell line panels. (E) Co-clustering CIM between the NCI-60 and the BLA-40 cell lines of the final 13 COXEN probes for paclitaxel (Supplemental Table S1). The sensitives (and resistants) of the NCI-60 and the BLA-40 cell lines were closely clustered together despite of their differences in their tissue origins. (F) Significance of COXEN biomarkers for BLA-40 sensitive and resistant cell lines to cisplatin and paclitaxel, respectively.

FIG. 3: Chemotherapeutic response prediction in patients with breast cancer: (A) Schematic diagram for COXEN based chemotherapeutic response prediction model development and model validation for breast cancer patients. (B) Direct comparison between the standardized MiPP predictive scores and the standardized patients' residual tumor sizes after mathematical standardization. The standardized scores were obtained by subtracting the overall mean divided by the standard deviation of the COXEN scores and the residual tumor sizes of the DOC-24. Statistical significance was determined using the Spearman correlation coefficient (p-value=0.022). (C) Kaplan-Meier survival curves for the COXEN predicted responder and nonresponder groups on the 60 breast cancer patients in the tamoxifen trial. The predicted responder group based on the top COXEN prediction model showed a significantly longer disease-free survival time than the predicted nonresponder group (G-rho family of survival tests; p-value=0.021). (D) Significance of COXEN biomarkers on the DOC-24 clinical trial of docetaxel and on the TAM-60 trial of tamoxifen, respectively.

FIG. 4: Human bladder cancer drug discovery and validation: (A) Schematic diagram for computation drug screening of 45,545 compounds in the public NCI database available at the NCI website (dtp.nci.nih.gov). (B) Effectiveness of NSC 637993 as a function of tumor histology of cancer cell lines is shown for the BLA-40 (Four cell lines are missing from panel due to difficulty growing them in culture). NSC 637993 is more effective at a lower dose (1×10⁻⁶M) in bladder cancer than that in the nine tissue-specific cell line panels of the NCI-60 cell lines. (C) Chemical structure of the lead novel compound NSC637993 discovered by COXEN.

FIG. 5: Chemotherapeutic response prediction in the BLA-40 bladder cell lines and the patients with breast cancer: Continuous performance of top three MiPP prediction models (A) on the BLA-40 sensitive and resistant cell lines for cisplatin. (B) on the BLA-40 sensitive and resistant cell lines for paclitaxel. (C) Responder and nonresponder patients in the docetaxel trial. (D) Responder and nonresponder patients in the tamoxifen trial. In these figures, each of the top three models showed consistent prediction performance for the corresponding cell lines and patients.

FIG. 6: Figures A to D, graphically illustrate the classification of sensitive and resistant cancer cell lines to single drug chemotherapy. (A) comprising six panels, illustrates growth-inhibition dose response curves for a) SLT4 and RT4 in respond to Cisplatin (upper two graphs); b) 253-JBV and RT4 to Paclitaxel (middle two graphs); and c) SW1710 and UMUC9 to Gemcitabine (lower two graphs. The left graphs of each group representative the sensitive cells and the right graphs of each group represent the resistant cells. The percent of cell counts (divided by 100) is indicated on the Y axis. Cell lines were defined as sensitive if GI50s were below the dose indicated by the vertical criterion line (CR), whereas resistant had GI50s above this dose. Cisplatin log 10(400 ng/ml), Paclitaxel log 10(0.005 uM), and Gemcitabine log 10(0.1 uM). Each individual experiment is indicated by a dotted line. The fitted nonlinear regression line (solid curve) represents the combined estimate. Determination of sensitive (S) and resistant (R) cell lines to (B) Cisplatin, (C) Paclitaxel and (D) Gemcitabine. log 10(GI30), log 10(GI50), and log 10(GI70) of the 40 cell lines are indicated by gray, green, and red, respectively.

FIG. 7: 2D scatter plots of expression intensities (log 2 scale) of the first two genes of single-drug prediction models demonstrating their classification performance. The genes listed are described in the examples: (7A) Cisplatin. (7B) Paclitaxel (7C) Gemcitabine. Sensitive cells are indicated by blue dots () and resistant cells are indicated by red stars (*) cell lines were found to be separated by the two selected genes although some of them were still misclassified. Some of the misclassified ones were better separated by the additional genes, so the mean ERs were 0.069, 0.051, and 0.096 for Cisplatin, Paclitaxel, and Gemcitabine, respectively.

FIG. 8: The scatter plot of the percent of cell counts compared to control (no drug) versus the posterior probability of sensitivity for the 15 cell lines randomly selected for the evaluation of chemotherapeutic sensitivity prediction for the three two-drug combinations shown. The horizontal (55%) and vertical (0.75) dotted lines divided cell lines into sensitive and resistant based on the percent of cell count and the posterior probability of sensitivity, respectively. The ordinate represents percent cell count and the abscissa represent probability of drug sensitivity. Abbreviations: Cis: Cisplatin, Pac: Paclitaxel and Gem: Gemcitabine.

FIG. 9: Classification of responder and nonresponder patients in the tamoxifen trial: Patients with recurrent disease had tumor recurrences within a relatively short time (<50 months) after the tamoxifen treatment, whereas no patient with durable survival falls in this time period. Hence, the assumption was made that such early recurrence patients were tamoxifen nonresponders (16 patients). In contrast, patients with long-term survival (>130 months) were considered responders (11 patients).

FIG. 10. In vitro drug chemosensitivity of NCI-60 and BLA-40 cell lines. (A) Ordered log(GI50) values of the NCI-60 cell line responses to cisplatin. (B) Ordered log(GI50) values of the NCI-60 cell line responses to paclitaxel. (C) Ordered log(GI50) values of the BLA-40 cell line responses to cisplatin. (D) Ordered log(GI50) values of the BLA-40 cell line responses to paclitaxel.

FIG. 11: Illustrated is the top-scoring pathway as defined by the Ingenuity analysis tool. Each pathway member is depicted by a symbol. Red symbols indicate those genes with down-regulated expression, green represents the genes with increased expression in the analysis, white symbols identifies pathway members not found altered in the tumor cells. (A) Ingenuity generated interaction pathways of the identified COXEN biomarkers of response for the DOC-24 breast clinical trial of docetaxel. (B) Ingenuity generated interaction pathways of the identified COXEN biomarkers of response for the human bladder cancer cell lines (BLA-40) to paclitaxel. (C) Ingenuity generated interaction pathways of the identified COXEN biomarkers of response for the human bladder cancer cell lines (BLA-40) to cisplatinum.

FIG. 12: Shows the COXEN combination chemosensitivity prediction on 43 lymphoma patients treated with CHOP-like regimen (cyclophosphamide, doxorubicin, vincristine, and prednisone).

### DETAILED DESCRIPTION OF THE INVENTION

The present invention encompasses a novel method for identifying the activity of an agent or combination of agents. The invention is achieved by the creation and use of an algorithm termed “CO-eXpression ExtrapolatioN” (COXEN). The algorithm uses specialized molecular profile signatures for translating an agent(s) sensitivity signature from one set of cells to that of another set of cells (e.g., translating data from the NCI60 panel to a panel of cells not present in the NCI60 panel).

The present invention provides a potential solution to major problems in drug development as well as in the selection of optimal therapeutic regimens (personalized medicine). That is, while thousands of agents have been and are being synthesized, there are essentially no generally reliable ways to predict which of those agents will be active against a disease or disease model or potentially effective as a therapeutic agent. Cell and animal models have not been useful in this regard. Hence, many useful agents end up neglected (“leaky pipeline”), while others are only found to fail after expensive and time-consuming clinical trials. Together, this results in a “status quo” where long drug development timelines and huge costs are the norm.

The methods of the present invention address the above problem in drug discovery by accurate prediction of an agent(s) effectiveness in patients from in vitro sensitivity experiments on cell sets using the presently disclosed “CO-eXpression ExtrapolatioN” (COXEN) technique. For clinical trials, the present invention has at least two applications: 1) selecting the optimal lead agents for Phase I human trials; and, 2) patient selection for Phase II and III clinical trials for agents that have already passed Phase I, markedly improving odds for success of these latter trials.

The present invention addresses the need for personalized medicine (or personalized selection of medicines) by accurate prediction of a single agent or combination of agents effectiveness in specific patients from in vitro agent sensitivity experiments on cell sets. The invention addresses the problem of how to select combinations of therapeutic agents with therapeutic effectiveness, thereby allowing the medical practitioner to select a combination of agents that will provide the highest combination-agent activities to specific patients. In essence matching the patients disease/tumor etc. to the ideal treatment comprised of a combination of agents.

The COXEN method provided herein is useful for: 1) extrapolating agent sensitivity data obtained from in vitro screening of a cell set to predict the sensitivity/response of cell lines and diseases (e.g., cancers, diabetes, etc.) to agents; and, 2) testing and identifying agents for their ability to act as therapeutic agents for diseases (e.g., cancers, diabetes, etc.).

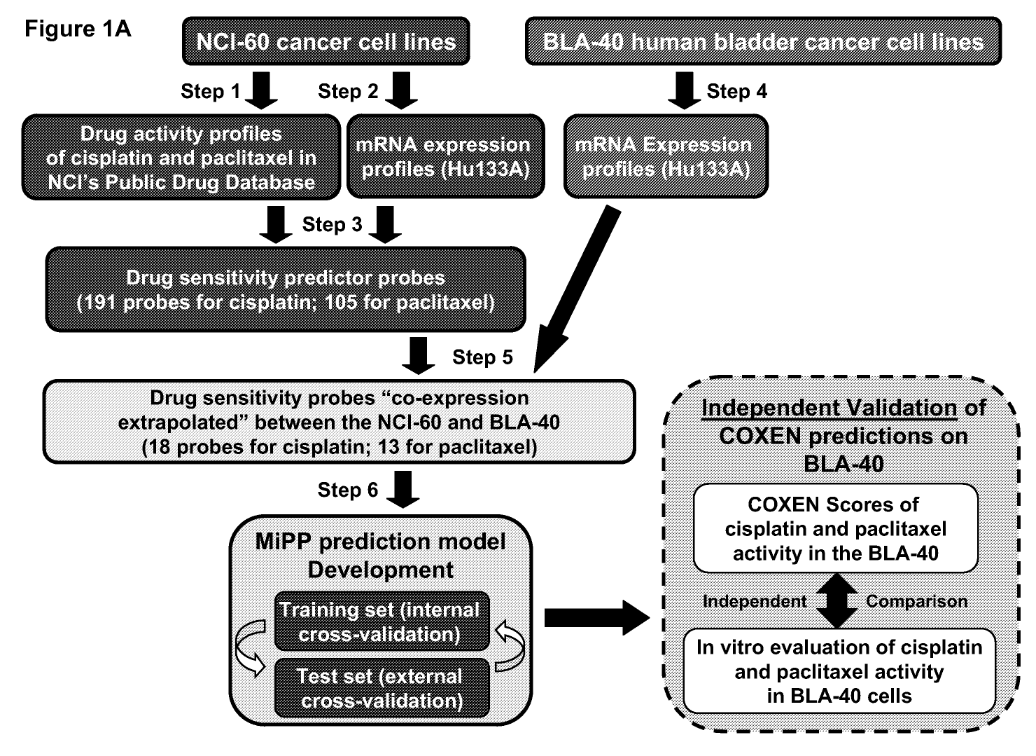

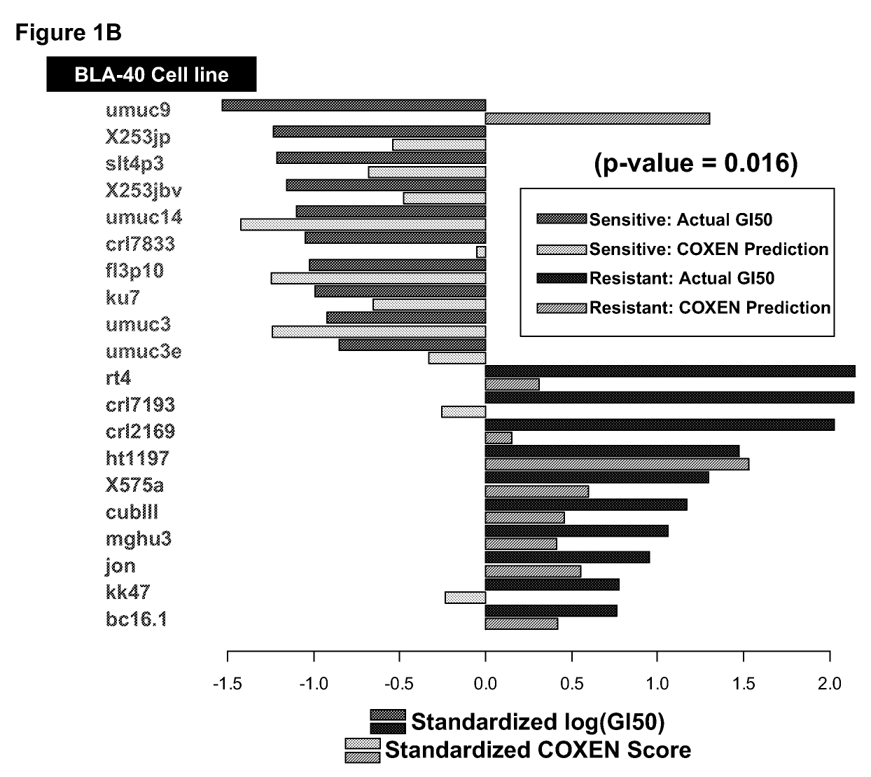

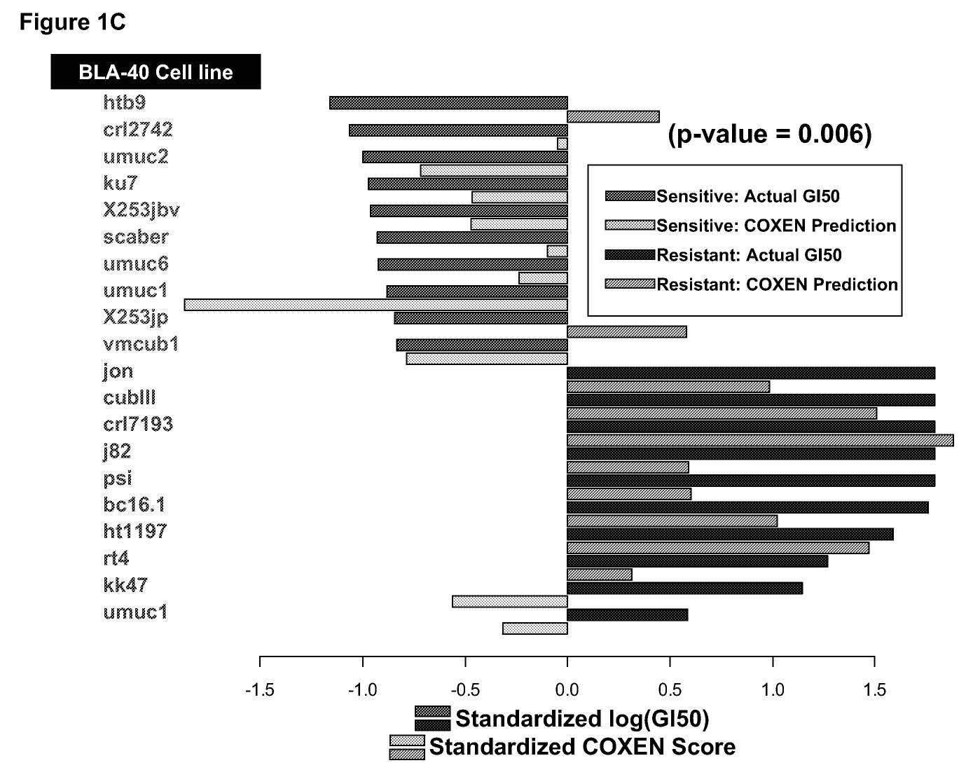

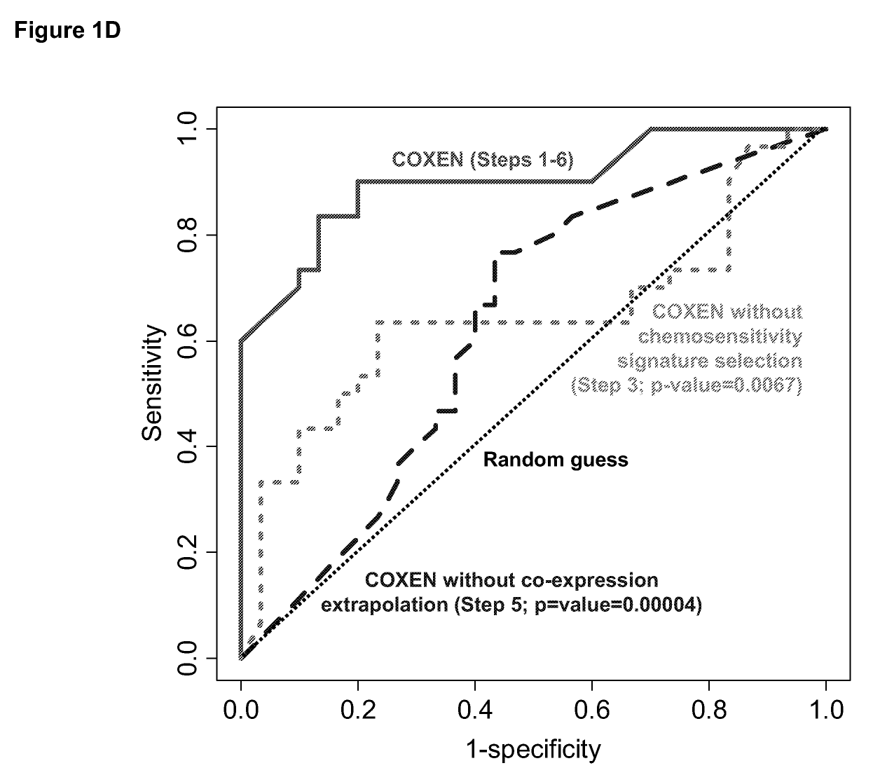

The basic protocol of the present invention is as follows (also see FIG. 1A):
(1) STEP 1: Determine an agent's pattern of activity in cells of set 1.
(2) STEP 2: Measure molecular characteristics of the cells in set 1.
(3) STEP 3: Select a subset of those molecular characteristics that most accurately predicts the agent's activity in set 1 (chemosensitivity or agent activity signature selection).
(4) STEP 4: Measure the same molecular characteristics of the cells in set 2.
(5) STEP 5: Identify a subset among the molecular characteristics selected in (3) that are concordant (i.e., show a strong pattern of “co-expression” or “co-association”) between sets 1 and 2. These molecular characteristics can be further reduced in number and data dimension by using a multivariate classification or dimension reduction algorithm.
(6) STEP 6: Use a multivariate classification algorithm to predict an agent's activity in set 2 cells using the trained classification model on the basis of the drug's activity pattern and the molecular characteristics in set 1 selected in (5) and applying the trained classification model to set 2 on the same molecular characteristics in set 2 selected in (5).
(7) Test the predictions prospectively by independent experiment (or using independent clinical response or outcome data).

The process described above can be modified (e.g., the independent testing can be omitted), and in some cases the order can be changed, without deviating from the spirit of the present invention.

The present invention provides a novel agent discovery methodology that was developed and validated in bladder cancer cells and breast cancer patients. The method is useful, for example, for virtual screening of the approximately 45,545 compounds in the NCI drug database, and providing a list of compounds for human bladder cancer with putative activity in this tumor. The method is also useful for screening other compounds and other diseases as well. Furthermore, the use of at least one of the compounds of the NCI drug database is validated herein for its effectiveness in human bladder cancer. This paradigm shifting approach will greatly accelerate anticancer drug discovery and clinical care of patients (e.g. for patients with cancer).

The utility of the present invention has been demonstrated using a series of 40 human urothelial cancer cell lines (BLA-40), measuring the growth inhibition elicited by three widely-used chemotherapeutic agents: cisplatin, paclitaxel, and gemcitabine in the BLA-40, and correlating these GI50 (50% of growth inhibition) values with quantitative measures of global gene expression on these cell lines. In silico prediction models of single-drug chemosensitivity were derived using a multivatiate classification/prediction algorithm, so-called misclassification penalized posterior (MiPP) approach. Combining these individual-drug chemosensitivity prediction models, a statistical method was then used to predict the cell lines' cellular growth responses to clinically relevant two-agent combinations. By virtue of using single drug sensitivities to mathematically predict combination effects (rather than using effects of combination directly), the present invention has the unique advantage of allowing the evaluation of any number of agents in combination and of allowing the integration of new agents into new combinations as needed.

In the present invention, at least two types of data sets are required, (a) a training set and (b) a validation set. The training set is comprised of compound activity data and molecular characteristic data from a first cell set. The activity data allows one to determine which cells (or patients) are resistant and which are sensitive to a tested agent (e.g., drug substance or compound from a library) or group of agents (e.g., all approved cancer drug substances or a compound library) and what molecular characteristics are related to this resistance and sensitivity. The validation set is comprised of molecular characteristics from a second, distinct cell set. By distinct, it is meant that the data of the validation set is derived from cells (or other sources) that may not be present in the training set (e.g., the second set is derived from a series of bladder cancer cell lines and the first set is the NCI60 panel). The validation set allows one to then select a set of molecular characteristics that are concordant to the training and validation sets. This concordant set of molecular characteristics allows one to then predict an agent's activity against the cells of the validation set.

The present invention can use a third or more cell sets to further improve predictive accuracy that an agent will be more effective in a certain situation, cell or patient. The source of the third or other additional cell sets is distinct from the first and second sets (e.g., human tissues for the third set and cell lines for the first and second cell sets). However, the disease state of the cells can be the same or different from the first and second sets (e.g., the third set can be derived from human bladder cancer tissues, the second from bladder cancer cell lines, and the first the NCI60 panel (which does not contain bladder cancer cells). For example, a set of molecular characteristics concordant to the first and third cell sets is determined (i.e., a second concordant set). A set of molecular characteristics common to the two concordant sets is then determined. This common set of molecular characteristics can then be used to predict the activity of the agents both against the second and the third cell sets without physically conducting the experiments. This dual prediction is particularly important in novel drug discovery. For example, one can determine new agent leads from a library of agents that have efficacy both on the second cell line set and the third human bladder cancer patient set. Once a lead agent is experimentally validated on the second cell line set, it has a high likelihood to be effective for the third human cancer patient set, which would not have been realized in the classical ways (current paradigms) until expensive human clinical trials has been performed. Thus, one can very efficiently discover and validate a drug or drugs that have the effectiveness against the disease of a patient, thereby significantly reducing the cost and risk of discovery of human therapeutic agents.

The present invention is useful for preparing and comparing molecular profiles for various kinds of cell sets. This information can be used in conjunction with current databases, or new databases, to predict the response of a test cell to an agent (e.g., a drug substance or a test compound).

In another embodiment, the present invention provides a novel method of treating a subject in need thereof with an agent identified by the methods of the invention.

In another embodiment, the present invention provides a novel method of predicting the effectiveness of a known agent in a patient in need of treatment. For example, a tissue sample from a cancer patient can be used in the present invention to determine what cancer agent(s) will be effective against that patient's tumor without having the patient's tumor ever exposed to the agent. In addition, the present invention can be used to determine what combination of agents will be effective against that patient's tumor without having the patient's tumor ever exposed to the agent.

In another embodiment, the present methods are useful for agent screening (e.g., cancer agent screening). Organizations such as the NCI and large pharmaceutical companies have been using the NCI-60 panel or similar panels to screen hundreds of thousands perhaps even millions of agents. This information can be used with the methods of the present invention to select top agents candidates for every single human tumor, even those tumors that are not on the specific panel used for the screen. Furthermore, the studies disclosed herein demonstrate how COXEN can be used in a screening mode and goes on to identify an agent that is potent and selective in bladder cancer.

In essence, combining the ability to predict effectiveness in patients with that of computational drug screening, will yield new agent candidates and new combinations of agent that have a high likelihood of being effective in patients with the disease studied (e.g., cancer). For example, the methods of the present invention are applicable for use in screening agent and agent combinations useful for treating any human tumor/cancer in patients.

The present invention further provides methods and compositions useful for therapeutic agent selection and discovery for patients with rare or orphan tumors. For example, most drug development and clinical trials in cancer have concentrated on common tumors. While this is understandable, many less common tumors have become “orphaned” and patients left without any guidance as to the optimal agents to use. Furthermore, few if any drug discovery efforts or clinical trials are being undertaken in these. The COXEN technique can be used to 1) generate lists of optimal agents to use in patients among agents currently FDA approved for cancer; 2) provide new agents among those where sensitivity of said agents in cell line, animal tumors, tissues or organs either syngeneic or xenograft or patient tumor responses is known; and, 3) predict which individuals will be responsive to these identified agents (i.e., personalized medicine).

In another embodiment, the present invention provides a novel method for predicting the activity of at least one agent, comprising:
(a) determining an agent's pattern of activity against a 1^{st} cell set (CS-1), wherein this activity determination shows which cells are sensitive and resistant to the agent;
(b) measuring a set of molecular characteristics (MC-1) for each cell represented in CS-1;
(c) selecting a subset of molecular characteristics (MC-2) from MC-1 for each cell represented in CS-1, each subset comprising: those molecular characteristics that most accurately predict the agent's activity against each cell represented in CS-1 (chemosensitivity or agent activity signature selection);
(d) measuring the same set of molecular characteristics (MC-3) as MC-1 for each cell represented in a 2^{nd} cell set (CS-2), wherein CS-2 contains cells that differ from those of CS-1;
(e) identifying a set of molecular characteristics (MC-4) that is a subset of MC-2 and MC-3, wherein MC-4, comprises: a set of molecular characteristics concordant to sets MC-2 and MC-3 (biomarker identification of concordantly-expressed or concordantly-associated (e.g., if SNP data is used) molecular networks between two different sets); and,
(f) predicting the agent's activity against each cell represented in CS-2, comprising: using a multivariate classification algorithm that compares the agent's determined activity against CS-1 with MC-4.

In another embodiment, the present invention provides a novel method, wherein step (f), comprises:
(f-i) prior to predicting the agent's activity against CS-2, using a multivariate algorithm to reduce the number of molecular characteristics of MC-4 to form MC-4A, comprising: evaluating different combinations and selecting the best combinations of the molecular characteristics in MC-4 with a multivariate classification algorithm for their overall prediction performance of the agent's activity against CS-1, or alternatively, combining the information in MC-4 with a multivariate dimension reduction algorithm to form MC-4A; and,
(f-ii) predicting the agent's activity against each cell represented in CS-2, comprising: using a multivariate classification algorithm that compares the agent's determined activity against CS-1 with MC-4A.

In another embodiment, the present invention provides a novel method, wherein the activity against CS-2 is estimated by observing how closely the molecular characteristics MC-4A of each cell in CS-2 match, in terms of the presence and expression levels of the same characteristics, the molecular characteristics MC-4A of the sensitive and resistant cells in CS-1.

In another embodiment, the present invention provides a novel method, wherein the method further comprises: replacing (f) with at least the following:
(g) measuring a set of molecular characteristics (MC-5) for each cell represented in a 3 cell set (CS-3), wherein CS-3 contains cells that differ from those of CS-1 and CS-2, which may differ by its source, e.g. in vitro vs. in vivo, or human patients vs. animal models; and;
(h) identifying a set of molecular characteristics (MC-6) that is a subset of MC-2 and MC-5, wherein MC-6, comprises: a set of molecular characteristics concordant to sets MC-2 and MC-5 (biomarker identification of concordantly-expressed or concordantly-associated molecular networks between MC-2 and MC-5);
(i) identifying a set of molecular characteristics (MC-7) that is a subset of concordant sets MC-4 and MC-6, wherein MC-7, comprises: a set of molecular characteristics common to sets MC-4 and MC-6 (biomarker identification of concordantly-expressed or concordantly-associated molecular networks across all three sets MC-2, MC-3 and MC-5);
(j) predicting the agent's activity against each cell represented in CS-2 and CS-3, comprising: using a multivariate classification algorithm that compares the agent's determined activity against CS-1 with MC-7.

In another embodiment, the present invention provides a novel method, wherein step (j), comprises:
(j-i) prior to predicting the agent's activity against CS-2 and CS-3, using a multivariate algorithm to reduce the number of molecular characteristics of MC-7 to form MC-7A, comprising: evaluating different combinations and selecting the best combinations of the molecular characteristics in MC-7 with a multivariate classification algorithm for their overall prediction performance of the agent's activity against CS-1, or alternatively, combining the information in MC-7 with a multivariate dimension reduction algorithm to form MC-7A; and,
(j-ii) predicting the agent's activity against each cell represented in CS-2 and CS-3, comprising: using a multivariate prediction algorithm that compares the agent's determined activity against CS-1 with MC-7A.

In another embodiment, the present invention provides a novel method, wherein the agent is from NCI-60 anticancer drug screening database.

In another embodiment, the present invention provides a novel method, wherein the activity against CS-2 and CS-3 is estimated by observing how closely the molecular characteristics MC-7A of each cell in CS-2 and CS-3 match, in terms of the presence and expression level of the same characteristics, those of sensitive and resistant cells in CS-1.

In another embodiment, the present invention provides a novel method, wherein the activity determined is the agent's cytostaticability (growth inhibition) and/or cytotoxicity (cell death) against each cell type in CS-1.

In another embodiment, the present invention provides a novel method, wherein each cell set is a cancer cell set and the activity being tested is anti-cancer activity.

In another embodiment, the present invention provides a novel method, wherein CS-1 is a panel of cancer cells.

In another embodiment, the present invention provides a novel method, wherein the panel of cancer cells is the NCI-60 panel.

In another embodiment, the present invention provides a novel method, wherein CS-2 is a set of cells derived from human laboratory cell lines.

In another embodiment, the present invention provides a novel method, wherein the human laboratory cell lines are cancer cell or endothelial cell lines.

In another embodiment, the present invention provides a novel method, wherein the type of cancer is selected from bladder, lung, brain, breast, liver, colon, rectal, melanoma, pancreatic, leukemia, non-Hodgkin lymphoma, kidney, endometrial, prostate, thyroid, meningiomas, mixed tumors of salivary glands, adenomas, carcinomas, adenocarcinomas, sarcomas, dysgerminomas, retinoblastomas, Wilms' tumors, neuroblastomas, ovarian, squamous cell carcinoma, pancreatic, and mesotheliomas.

In another embodiment, the present invention provides a novel method, wherein wherein CS-3 is a set of cells derived from human tissue samples.

In another embodiment, the present invention provides a novel method, wherein the human tissue samples were taken from cancerous tissues.

In another embodiment, the present invention provides a novel method, wherein the type of cancer is selected from bladder, lung, brain, breast, liver, colon, rectal, melanoma, pancreatic, leukemia, non-Hodgkin lymphoma, kidney, endometrial, prostate, and thyroid.

In another embodiment, the present invention provides a novel method, wherein CS-3 is a set of cancer cells derived from human tissue samples of the same type of cancer as that of CS-2.

In another embodiment, the present invention provides a novel method wherein the molecular characteristics are selected from (i) profiling of gene expression, (ii) profiling of SNPs (single nucleotide polymorphisms), (iii) profiling of protein expression

In another embodiment, the present invention provides a novel method, wherein the molecular characteristics are mRNA expression profiles.

In another embodiment, the present invention provides a novel method, wherein the agent is at least one pharmaceutically active ingredient (API), at least one cancer API, or a group of APIs corresponding to all FDA approved cancer APIs.

In another embodiment, the present invention provides a novel method, for selecting a patient-specific API, comprising:
(a) determining each API's pattern of activity against a 1^{st} cell set (CS-1), wherein this activity determination shows which cells are sensitive and resistant to the API;
(b) measuring a set of molecular characteristics (MC-1) for each cell represented in CS-1;
(c) selecting a subset of molecular characteristics (MC-2) from MC-1 for each cell represented in CS-1, each subset comprising: those molecular characteristics that most accurately predict the API's activity against each cell represented in CS-1;
(d) measuring a set of molecular characteristics (MC-3) for a patient's tissue sample (TS-1), wherein the patient is in need of therapy;
(e) identifying a set of molecular characteristics (MC-4) that is a subset of MC-2 and MC-3, wherein MC-4, comprises: a set of molecular characteristics concordant to sets MC-2 and MC-3;
(f) using a multivariate classification algorithm to reduce the number of molecular characteristics of MC-4 to form MC-4A, comprising: evaluating different combinations and selecting the best combinations of the molecular characteristics in MC-4 with a multivariate classification algorithm for their overall prediction performance of the API's activity against CS-1, or alternatively, combining the information in MC-4 with a multivariate dimension reduction algorithm to form MC-4A; and,
(g) creating prediction models, comprising: using a multivariate classification algorithm to predict each API's activity against CS-1 with MC-4A;
(h) predicting each API's activity against TS-1 using MC-4A in the prediction models.

In another embodiment, the present invention provides a novel method, wherein the activity against TS-1 is estimated by observing how closely the molecular characteristics MC-4A of each cell in TS-1 match, in terms of the presence and expression levels of the same characteristics, those of sensitive and resistant cells in CS-1.

In another embodiment, the present invention provides a novel method, wherein CS-1 corresponds to the set of NCI-60 cancer cell lines or a similar set of cancer cell line panels.

In another embodiment, the present invention provides a novel method, wherein CS-1 corresponds to a set of patients and the data for (a) and (b) are collected from the response data and patient microarray data of the patients.

In another embodiment, the present invention provides a novel method, wherein the patient response data and microarray data are from patients who have received therapy for a cancer or other disease.

In another embodiment, the present invention provides a novel method, wherein the method further comprises:
(i) repeating steps (a)-(h) for a group of APIs resulting in a data set of each API's activity against TS-1 as well as a sensitivity and resistance characteristics against CS-1;
(j) selecting first set of combinations of at least 2 APIs by comparing their predicted activities (i.e., individual predicted probabilities of sensitivity) against TS-1 with their known molecular mechanisms and toxicities to arrive at highly active combinations whose expected toxicity levels are tolerable to the patient;
(k) selecting a second set of combinations, wherein the second set if a subset of the first set of combinations, the second set being selected by choosing those combinations whose individual API sensitivity and resistance characteristics are the least correlated;
(l) predicting the combined activities of the second set of combinations of APIs in two ways, (I) assuming those APIs' activities are independent or (II) assuming their activities are correlatively additive on the basis of the sensitive and resistance characteristics on CS-1.

In another embodiment, the present invention provides a novel method, of treating cancer, comprising: administering a therapeutically effective amount of a compound of Table 3, 4, 5, 6, or 7 or a pharmaceutically acceptable salt thereof, wherein the cancer is selected from breast, bladder, prostate, melanoma, and pancreatic.

In another embodiment, the present invention provides a novel hardware device, comprising: a machine readable storage device have stored thereon a computer program, comprising: a plurality of code sections executable by a machine for performing a process as described herein.

In another embodiment, the present invention provides a novel method for predicting the activity of at least one agent, said method comprising: a hardware device having a machine readable storage, having stored thereon a computer program comprising a plurality of code sections executable by a machine, for performing the steps described herein.

In another embodiment, the methods of the present invention can be used for determining toxicity profiles of agents used or in development for human disease. For example, by applying the COXEN technology between sets of cancer cells or other cells exposed to agents in vitro and normal cells or tissues, one could predict the toxicity profile of the various compounds in patients without the use of animal models.

One of ordinary skill in the art will also appreciate that the methods of the present invention are useful for screening compounds from any source, including such sources as plants, animals, herbs, and their extracts, and libraries of compounds not disclosed herein.

The invention also encompasses the use of pharmaceutical compositions to practice the methods of the invention, the compositions comprising an appropriate compound, or an analog, derivative, or modification thereof, and a pharmaceutically-acceptable carrier.

The pharmaceutical compositions useful for practicing the invention may be administered to deliver a dose of between 1 ng/kg/day and 100 mg/kg/day.

Pharmaceutical compositions that are useful in the methods of the invention may be administered systemically in oral solid formulations, ophthalmic, suppository, aerosol, topical or other similar formulations. Such pharmaceutical compositions may contain pharmaceutically-acceptable carriers and other ingredients known to enhance and facilitate drug administration. Other possible formulations, such as nanoparticles, liposomes, resealed erythrocytes, and immunologically based systems may also be used to administer an appropriate agent according to the present invention.

Compounds that are identified using any of the methods described herein may be formulated and administered to a mammal for treatment of a disease described herein.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal, venous, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. “Unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion. An “oily” liquid is one which comprises a carbon-containing liquid molecule and which exhibits a less polar character than water.

“Parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including suspending, stabilizing, or dispersing agents. In one embodiment of a formulation for parenteral administration, the active ingredient is provided in dry (i.e. powder or granular) form for reconstitution with a suitable vehicle (e.g. sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non toxic parenterally acceptable diluent or solvent, such as water or 1,3 butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi liquid preparations such as liniments, lotions, oil in water or water in oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

Typically, dosages of the compound of the invention which may be administered to an animal, preferably a human, range in amount from 1 μg to about 100 g per kilogram of body weight of the animal. While the precise dosage administered will vary depending upon any number of factors, including the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, including the type and severity of the disease being treated, the type and age of the animal, etc.

The present invention also includes a kit comprising the composition of the invention and an instructional material which describes administering the composition to a cell or a tissue of a mammal. In another embodiment, this kit comprises a (preferably sterile) solvent suitable for dissolving or suspending the composition of the invention prior to administering the compound to the mammal.

The present invention further provides kits for use in administering or using compounds of the present invention.

The present invention may be embodied in other specific forms without departing from the spirit or essential attributes thereof. This invention encompasses all combinations of aspects of the invention noted herein. It is understood that any and all embodiments of the present invention may be taken in conjunction with any other embodiment or embodiments to describe additional embodiments. It is also to be understood that each individual element of the embodiments is intended to be taken individually as its own independent embodiment. Furthermore, any element of an embodiment is meant to be combined with any and all other elements from any embodiment to describe an additional embodiment.

The examples provided in the definitions present in this application are non-inclusive unless otherwise stated. They include but are not limited to the recited examples.

API: active pharmaceutical ingredient (aka, drug substance);

CEEC: co-expression extrapolation coefficient;

CIM: co-clustering cluster image map;

COXEN: COeXpression ExtrapolatioN;

MiPP: misclassification-penalized posterior;

ROC—receiver-operator characteristics

Examples of multivariate classification/prediction algorithms include algorithms selected from linear discriminant analysis (LDA), quadratic discriminant analysis (QDA), support vector machine (SVM), gene voting, logistic regression classification, neural network classification, CART classification, MiPP, and classical and Bayesian regression modeling, regression-tree classification, and random forest classification.

Examples of multivariate dimension reduction algorithms include algorithms selected from principal component analysis and singular value decomposition.

The articles “a” and “an” refer to one or to more than one, i.e., to at least one, of the grammatical object of the article. By way of example, “an element” means one element or more than one element.

The term “about” means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Concordant, with respect to molecular characteristics, means that a particular molecular characteristic behaves similarly in terms of association with other molecular characteristics of interest between two different cell sets.

Agent includes a pharmaceutically active ingredient (API) or drug substance (i.e., the active ingredient of drug product that has been approved for human use by an appropriate agency (e.g., the Food and Drug Administration in the United States)). Agent also includes a compound that is a potential drug substance or a potential lead compound in the search for a drug substance. Examples of APIs include cancer APIs (e.g., all FDA approved cancer APIs). Agent also includes a library of compounds (e.g., a group of compounds used to screen for research leads). A library of compounds can include 10, 100, 1,000, 10,000, or more compounds.

“Compound” refers to any type of substance that is commonly considered a chemical, biological (e.g., protein), drug, or a candidate for use as a therapeutic agent for use in a mammal (e.g., human). The source of the compound can be natural (e.g., a natural product), synthetic (e.g., a man-made API), or semi-synthetic (e.g., a modified natural product).

“Cell set” includes groups (e.g., panels) of cells and/or tissues. Thus, when cells are referred to in the claims, tissues are also included. The cells and tissues can come from a variety of sources including cell lines and tissue samples (e.g., tissues from a patient or patients). Cell set also includes a group of patients (e.g., patient set) whose molecular characteristics and sensitivity or resistance to an API have previously been determined (e.g., publicly reported).

The cell sets are typically representative of a disease state (e.g., cancer or diabetes) and can be various cells of one type of disease (e.g., various bladder cell lines) or various cells of different types of the same disease (e.g., the NCI60 panel which contains cells of a wide variety of cancer types). Cell sets also include cell lines and/or cell tissues derived from normal (i.e., non-diseased) human samples (e.g., endothelial cells, white blood cells, and other marrow components).

An example of a panel of cancer cells is the NCI60 panel. Other similar panels would also be useful in the present invention.

Molecular characteristics are measurements of molecular components expressed and the levels of expression.

Molecular characteristics include profiling of (i) gene expression, (ii) SNPs (single nucleotide polymorphisms), (iii) protein expression (i.e., proteomics and mass spectrometry), and (iv) any other genome-wide molecular characteristic(s) that can show different patterns between cells that are sensitive and resistant to an agent.

The determining of each agent's pattern of activity against a 1^{st} cell set can be accomplished experimentally or, when available, by using data from a database (e.g., selecting data from a published database). The data sought is the type that shows which cells are sensitive and resistant to the agent. When more than one agent is being tested, this activity data will need to be determined for each agent.

One of ordinary skill in the art can take advantage of published data when determining a agent's pattern of activity and measuring a set of molecular characteristics. For example, there is microarray data available for cancer patients who have received cancer therapy. This data can be used to measure molecular characteristics. There is also data available showing patient response to treatment with a drug substance. For example, there is patient response data for cancer agents. This data can be used to determine whether or not a patient is sensitive or resistant to a specific agent. Thus, there is publicly available data showing the molecular characteristics of patients that are sensitive or resistant to an agent (e.g., a cancer drug).

Chemosensitivity signature selection means selecting a subset of molecular characteristics that most accurately predict an agent's activity against each cell represented in a cell set.

Examples of agent activity signature selection involve selecting 2, 3, 4 5, 6, 7, 8, 9, 10, 20, 30, 40, 50, 100, 150, 200, 250, and 300 gene expression biomarkers.

“Cancer” is defined as proliferation of cells whose unique trait-loss of normal controls—results in unregulated growth, lack of differentiation, local tissue invasion, and metastasis.

An “effective amount” means an amount of a compound or agent sufficient to produce a selected or desired effect. The term “effective amount” is used interchangeably with “effective concentration” herein.

“Pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

“Treating” or “treatment” covers the treatment of a disease-state in a mammal, and includes: (a) preventing the disease-state from occurring in a mammal, in particular, when such mammal is predisposed to the disease-state but has not yet been diagnosed as having it; (b) inhibiting the disease-state, i.e., arresting it development; and/or (c) relieving the disease-state, i.e., causing regression of the disease state until a desired endpoint is reached. Treating also includes the amelioration of a symptom of a disease (e.g., lessen the pain or discomfort), wherein such amelioration may or may not directly affect the disease (e.g., cause, transmission, expression, etc.).

“Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 1, 2-ethanedisulfonic, 2-acetoxybenzoic, 2-hydroxyethanesulfonic, acetic, ascorbic, benzenesulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methanesulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, and toluenesulfonic.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are useful. Lists of suitable salts are found in Remington's Pharmaceutical Sciences, 18th ed., Mack Publishing Company, Easton, Pa., 1990, p 1445, the disclosure of which is hereby incorporated by reference.

“Therapeutically effective amount” includes an amount of a compound of the present invention that is effective when administered alone or in combination to treat an indication listed herein. “Therapeutically effective amount” also includes an amount of the combination of compounds claimed that is effective to treat the desired indication. The combination of compounds can be a synergistic combination. Synergy, as described, for example, by Chou and Talalay, Adv. Enzyme Regul. 1984, 22:27-55, occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased effect, or some other beneficial effect of the combination compared with the individual components.

“Instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviation the diseases or disorders in a cell or a tissue of a mammal. The instructional material of the kit of the invention may, for example, be affixed to a container which contains the peptide of the invention or be shipped together with a container which contains the peptide. Alternatively, the instructional material may be shipped separately from the container with the intention that the instructional material and the compound be used cooperatively by the recipient.

### EXAMPLES

The invention is now described with reference to the following examples. These examples are provided for the purpose of illustration only and the invention should in no way be construed as being limited to these examples, but rather should be construed to encompass any and all variations which become evident as a result of the teachings provided herein.

MATERIAL AND METHODS: Below we will provide the materials and methods for COXEN use for single and combination agents. These sections are kept separate for clarity here, but in practice, will be used in an integrated and inter related manner to provide information.

Material and Methods (Single Agents)

Drug activity and transcript expression profile data (Steps 1, 2, and 4, FIG. 1A). Publicly available drug sensitivity data, expressed in terms of 50% growth inhibition (GI50) for the NCI-60 were obtained from the NCI DTP web site (dtp.nci.nih.gov). NCI-60 transcript expression profiles were previously generated in a collaboration between the NCI Genomics & Bioinformatics Group and GeneLogic, Inc. (Gaithersburg, Md., U.S.A.) using HG-U133A GeneChip® arrays (Affymetrix, Santa Clara, Calif., USA). BLA-40 transcript expression data were obtained using the HG-U133A chips as part of the present study (Supplementary Materials and Methods). We obtained and organized publicly available gene expression profiles for the clinical breast cancers, including HG-U95Av2 GeneChip® data for the 24 docetaxel trial patients and 22,575-gene customized cDNA array data for the 60 tamoxifen trial patients. We performed quality control checks on the Affymetrix array data for the NCI-60 and breast cancer patients and then analyzed them using the RMA algorithm to obtain expression levels. We analyzed the customized cDNA array data using in-house analysis tools principally written in R and then matched the resulting gene-level data with results from the HG-U133A arrays using annotation information provided in the original study.

Identification of candidate “chemosensitivity biomarkers” in the NCI-60 panel (Step 3). For each compound in the public NCI-60 drug database, we identified the approximately 20% of the NCI-60 cells most sensitive to the compound and the 20% most resistant. Using slightly different percent cutoffs did not change the ultimate results appreciably (data not shown). For concreteness in describing the COXEN algorithm and its results, we used the examples of cisplatin and paclitaxel in the NCI-60 drug database, two drugs commonly used for clinical treatment of human bladder cancer (Calabro et al., 2002). After selection of sensitive and resistant cells, we used the “Significance Analysis of Microarrays” (“SAM”) (Tusher et al., 2001, PNAS) or two-sample t-tests, the latter effectively equivalent to the former, with false discovery rate (FDR) 0.1 to identify microarray probe sets differentially expressed between the two cell subsets. Instead of using statistical testing for differences in molecular characteristics between selected sensitive and resistant cells, chemosensitivity biomarkers can be selected by evaluating overall correlation between each molecular characteristic and agent activity values, e.g., GI50. That procedure identified 191 probe sets for cisplatin and 105 for paclitaxel. Those probe sets can be thought of as candidate “chemosensitivity biomarkers” based on the NCI-60 data.

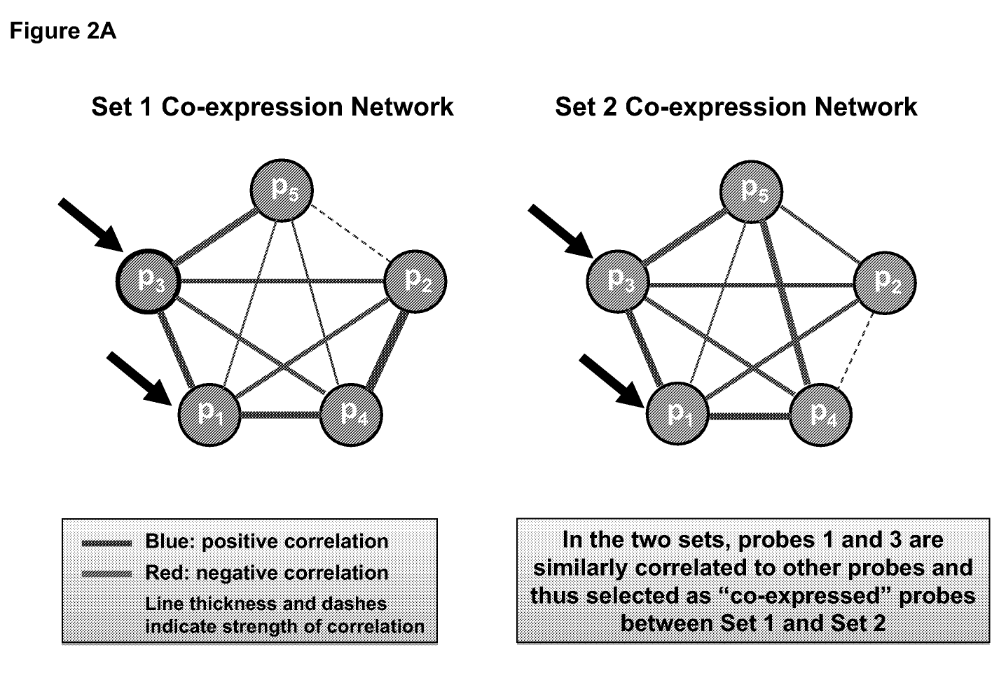

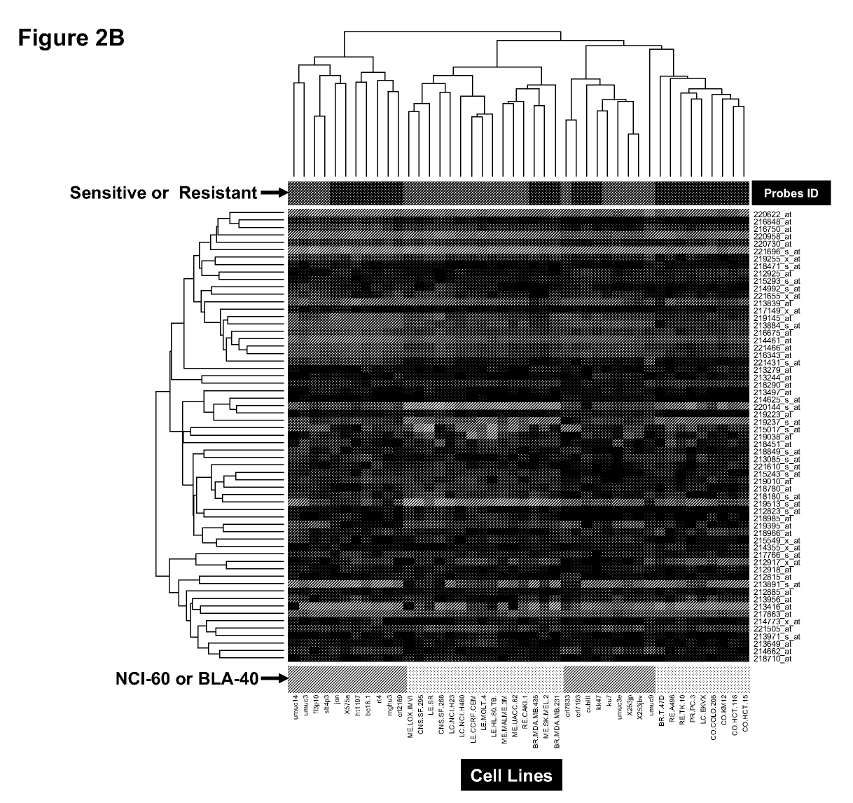

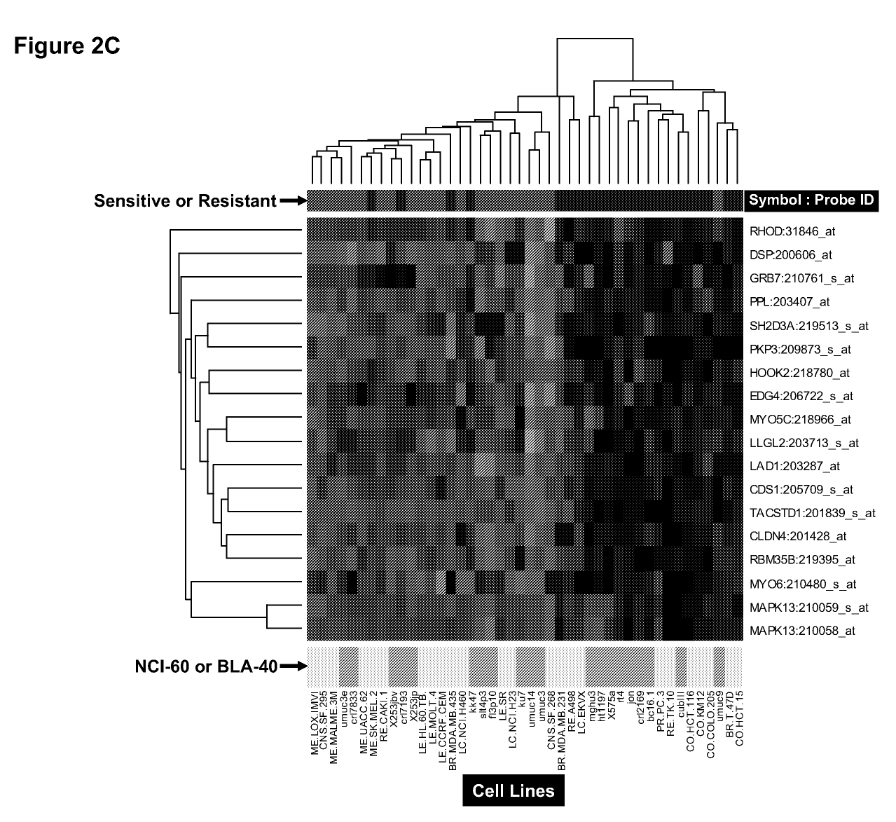

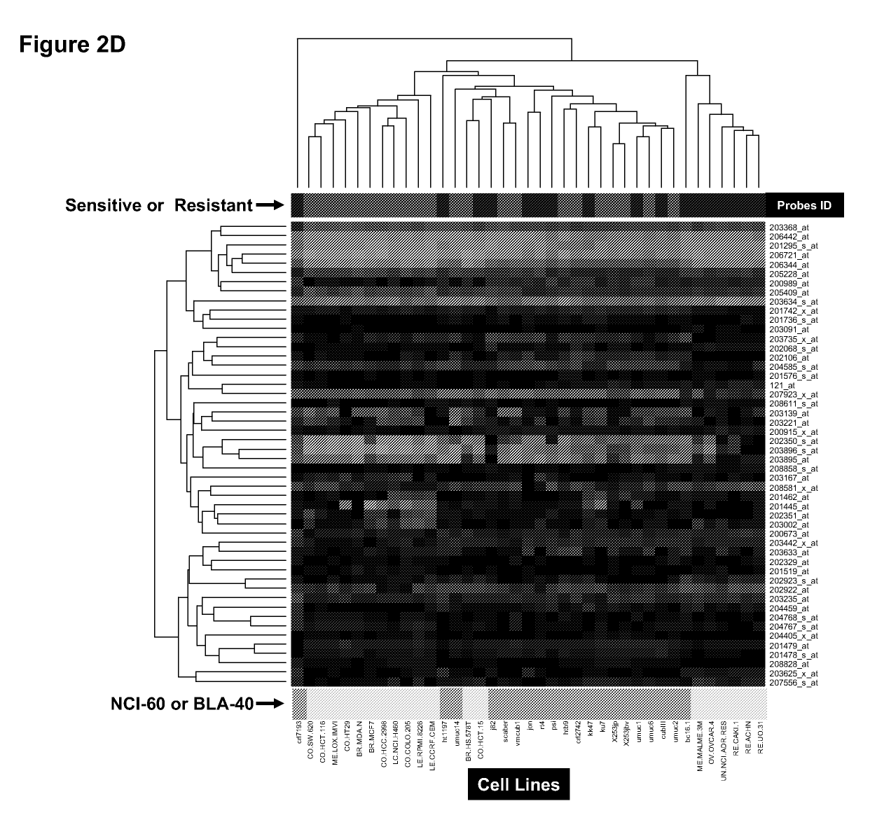

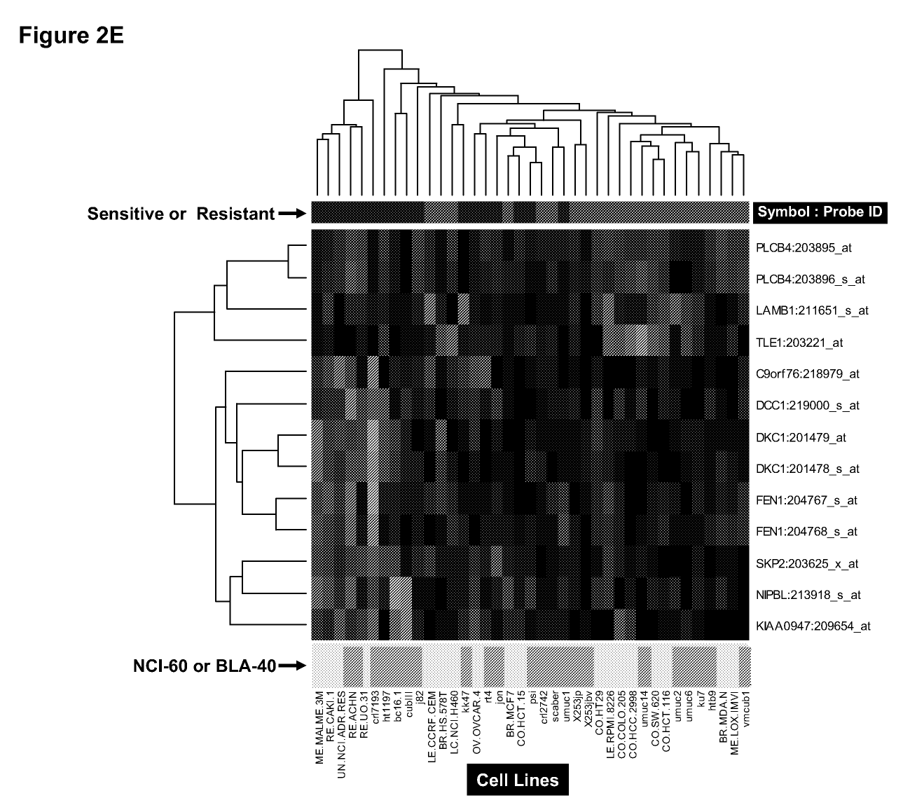

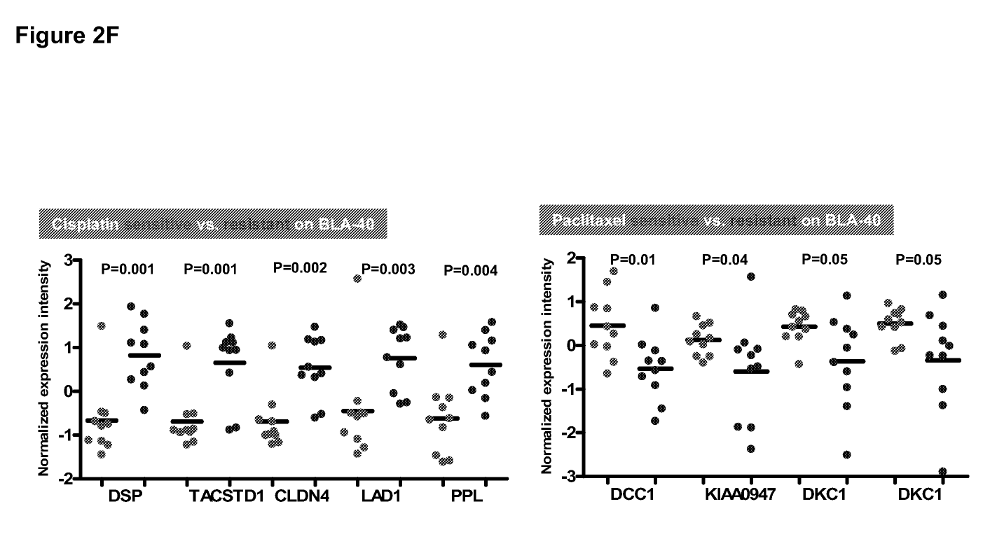

Identification of co-expression extrapolation signatures (Step 5) The co-expression extrapolation procedure is conceptually illustrated in FIG. 2A. Each gene's concordant co-expression relationships between two studies can be mathematically evaluated by co-expression extrapolation coefficient (CEEC). This CEEC will be high if a probe' co-expression network relationships with the other genes on the first set (i.e. NCI-60) are concordant with those of the second set (i.e., BLA-40). For example, applying this procedure to the 191 and 105 probe sets in FIG. 1A, 18 and 13 probe sets showed statistical significance (at p<0.02 one-tailed correlation distribution) for cisplatin and paclitaxel, respectively (Supplemental Table S1). These COXEN signatures can be further reduced in number and dimension by using multivariate classification or dimension reduction algorithms on the training set such as NCI-60.

Development of chemosensitivity prediction models for the NCI-60 panel (Step 6) We had identified candidate biomarker genes for each tested compound on the basis of significant differential expression for drug sensitivity in the NCI-60 and high CEEC between the NCI-60 and each of the target sets as described above. Next, we searched among those candidate biomarkers for ones that would form optimal parsimonious models for prediction of the compound's activity. For that purpose, we used the “Misclassification-Penalized Posterior” (MiPP) algorithm, which we introduced previously. This technique is described more in detail in Supplementary Materials and Methods.

Sensitivity of human bladder cancer cells to cisplatin, paclitaxel and NSC 637993. To test the predictive models, we performed in vitro drug response experiments, and then determined GI50 values for each bladder cell line for cisplatin, paclitaxel, and compound NSC 637993 (Supplementary Materials and Methods). Sensitivity to the agents was generated by a dose response experiments carried out on the BLA-40 cells as described for the NCI-60. The final concentrations of cisplatin used were 200, 400, 800, 1600, 3200, and 6400 ng/ml; those of Paclitaxel and NSC 637993 were 0.1, 1, 2, 5, 10, and 100 nM. In each case, the cells were plated on Day 0, exposed to drug for 48 hours) at 37° C., and then assayed. Each experiment was repeated three to five independent times, and the results were expressed as a fraction of the difference between initial cell count and untreated control. Log 10(GI50) values were then estimated from the resulting dose-response curves. Bladder cell lines were defined as sensitive or resistant as described above for the NCI-60 panel. Note that we had to use the NCI-60 activity data from another taxane, paclitaxel, rather than docetaxel itself, because complete docetaxel drug response data were not available in the NCI-60 database.

Discovery of novel candidate anticancer compounds from the NCI-60 screening data. To identify candidates in the NCI public database of 45,545 compounds that might be active against bladder cancer cells, we applied our COXEN computational screening algorithm with several additional filtering criteria. First, compounds with flat activity profiles across the NCI-60 were eliminated. Mathematically this was defined by the slope coefficient estimate from a simple linear regression for each drug compound. Second, the top and bottom 20% of cell lines were defined as “sensitives” and “resistants” of the NCI-60 panel for each compound. Third, we excluded the compounds that did not provide a good number (>10 or more) of statistically significantly (two-sample t-test FDR<0.1) differentially expressed probe sets between the resistant and sensitive cell line groups.

Material and Methods (Combination Agents)

Cell lines, Cell culture, Gene Expression Profiling and Dose Response Data Generation and Analyses for Combination Drug Prediction

The human bladder cancer cell lines and the respective growth conditions used in this study have been previously described (6, 7). Cisplatin was purchased from Sigma (St. Louis, Mo.), dissolved in Dulbecco's phosphate-buffered saline, and aliquoted in 1 mg/ml stocks. Paclitaxel was purchased from Sigma (St. Louis, Mo.), dissolved in Dimethyl Sulphoxide (DMSO), and aliquoted in 1 mM stocks. Gemcitabine was purchased from the University of Virginia Medical Center Pharmacy, dissolved in PBS, and aliquoted in 0.1 M stocks. Cell lines were maintained in appropriate media, in a humidified atmosphere containing 5% CO2 in air, except CRL2169 (SW780) which requires no CO2 for its growth. Cell lines were subcultured in an aqueous solution of 0.05% trypsin (Difco, 1:250) and 0.016% EDTA. Each cell line was used within 10 passages from its archival passage number in order to minimize any long term cell culture effects. Gene expression analysis of bladder cell lines was carried out as previously described using the HG-U133A GeneChip® array (Affymetrix®, Santa Clara, Calif., USA) (6, 7). The image file was analyzed with RMA, to obtain the expression intensity values of the microarray data (8).

Cell lines were seeded in 96-well cell culture plates (Costar) at a density of 1000 cells/well. 24 hours later, cells were exposed to the drugs diluted in RPMI-1640 medium, containing 10% FBS, concentration that is required by more than 75% of cell lines for their normal growth, at a total volume of 200 μL. Each drug dose was plated in triplicate, and the experiment was repeated four to seven times. The doses for Cisplatin were 200, 400, 800, 1600, 3200, and 6400 ng/ml; for Paclitaxel 0.0001, 0.001, 0.002, 0.005, 0.01, and 0.1 μM; for Gemcitabine 0.001, 0.01, 0.1, 1, 10, 100 μM. Plates were incubated for 72 hours with carrier or drug and growth inhibition was assessed by Alamar Blue (BioSource International, Inc Camarillo, Calif. (9, 10). Our doses for Cisplatin, Paclitaxel, and Gemcitabine were chosen to be similar to the range of doses used by NCI in their screening of the NCI-60 set of cell lines (http://dtp.nci.nih.gov).

Estimation of GI50 Values

From the dose-response data, log 10(GI50) values (log base 10 of concentration required to inhibit cell growth by 50% in comparison with untreated control) were estimated for all the cell lines by deriving log(dose) concentration curves on cell count percents as described below. To estimate the GI50 values reliably, we computed Euclidean distances among all replicated experiments, and excluded outlying experiments if they were in the top 20% among all measured distances. This percent was determined heuristically based on the general observations in experimental quality control. Furthermore, we did not see significant changes in our results by slightly changing this proportion as several replicated experiments were averaged to estimate our GI50 values (data not shown). Subsequently, the data were fitted to a sigmoidal function such as the following nonlinear regression model for estimating each cell line's dose response curve:

Percent=1−1/(1+exp(−(log 10(dose)−β)/α),

where α and β determine the shape of a fitted line.

This sigmoidal regression function was used to capture the natural shapes of drug dose responses. Thus, the estimated β is the predicted log 10(GI50) value, the expected log concentration achieving the cell count reduction of 50%. Similarly, log 10(GI30), and log 10(GI70) values, i.e. the concentrations required to inhibit cell growth by 30%, and 70% in comparison with untreated control, were also calculated.

Determination of Sensitive and Resistant Cell Lines for Single Drug Sensitivity

Cell line drug sensitivity was classified using the GI estimates and application of a criterion dose (CR) concept. We defined the CR as the minimum log 10(drug dose) among each compound's experimental dose concentrations at which at least 25% of the cell lines showed growth inhibition >50%. CRs were determined as log 10(400 ng/ml) for Cisplatin, log 10(0.005 μM) for Paclitaxel, and log 10(0.1 μM) for Gemcitabine, which provided at least 10 drug “sensitive” cell lines for each drug. Using these CR concentrations, each cell line was defined as sensitive if log 10(GI50)≦CR; strongly sensitive if log 10(GI30)≦CR, or resistant if log 10(GI70)>CR, and intermediate if log 10(GI50)>CR and log 10(GI70)<CR.

Statistical Discovery of Molecular Chemosensitivity Prediction Models for Single Drugs

For statistical discovery of prediction models, all 22,215 genes on the HG-U133A array were first evaluated for their ability to differentiate sensitive and resistant cell lines; intermediate lines were excluded from the analysis. The most significant genes were selected both by Local Pooled Error (LPE) test (11) and Significance Analysis of Microarrays (SAM) method (12). After candidate biomarker probes were identified for each tested compound on the basis of significant differential expression for drug sensitivity, we next searched among those candidate biomarkers for ones that would form optimal parsimonious models for prediction of the compound's activity. For this, we used the “Misclassification-Penalized Posterior” (MiPP) algorithm, which we introduced previously and is available at the open-source Bioconductor web site (www.bioconductor.org) (13). MiPP is based on stepwise incremental classification modeling discovery for the optimal, most parsimonious prediction models and double cross-validated evaluation for each trained prediction model. Model training can be performed from several different classification modeling techniques such as linear discriminant analysis (LDA), quadratic discriminant analysis (QDA), support vector machines (SVMs), or logistic regression; LDA was used for most application in our current study. In the double cross-validation, the first cross-validation is based on random splitting of the whole data set into a training set and an independent test set for external model validation; and the second is an n-fold cross-validation on the training set to avoid the pitfalls of a large-screening search and to obtain the most parsimonious optimal prediction models. Independent splits of the data result in multiple prediction models. MiPP generates multiple independent splits, which, in turn, results in multiple prediction models. The multiple models from different splits were re-evaluated on a large number of (e.g. 100) random splits of test and training sets to obtain their objective confidence bounds with the summary index, so-called sMiPP (standardized MiPP score), which varies between −1 to 1, from the worst to the best. From this confidence interval evaluation, mean and lower 5% sMiPP scores were obtained for each of the candidate prediction models, together with mean misclassification rates (ER). The final prediction of sensitive (or resistant) cell lines was performed by averaging its (posterior) classification probabilities of the top three prediction models exceeding 5% sMiPP>0.5. In performing MiPP analysis, we used the default values for many tuning parameters of the MiPP Bioconductor R package. For example, n.fold, p.test, n.split, and n.seq were 5, ⅓, 20, and 3, respectively. However, we pre-selected the most significant top 1% genes by LPE and SAM, and did not use the MiPP gene selection option by setting percent.cut=0.

Statistical Chemosensitivity Prediction for Combination Drug Treatments

Prediction of combination drug efficacy was obtained based on the final single-drug prediction models, directly utilizing each cell line's classification probabilities from these models. That is, assuming two different drug compounds acted independently, the combination chemosensitivity probability PAB of their combination treatment was derived as:

1−PA[resistant for drug A]×PB[resistant for drug B].

Here PA and PB are the chemosensitivity response probabilities based on the prediction models for compound A and B, respectively. Since this provides a somewhat optimistic probability evaluation of chemosensitivity, e.g., if PA=PB=0.5, then PAB=0.75, we used a strict decision criterion, PAB>=0.75 for predicting each cell line's chemosensitivity to combination treatment.

RESULTS: Below we will provide the results using COXEN for single and combination agents. These sections are kept separate for clarity here, but in practice, will be used in an integrated and inter related manner to provide information.

RESULTS (SINGLE AGENTS): To describe the use and demonstrate the capability of COXEN, three proof-of-principle test applications were addressed for single agents. First, a panel of 40 human urothelial bladder carcinomas (BLA-40) was assembled, profiled at the mRNA level as had been done with NCI-60, and the mRNA profiles of the two cell line panels were used to obtain a COXEN “Rosetta Stone” profile for prediction of drug sensitivities of the BLA-40 from those of the NCI-60. Second, response and disease-free survival data were used from clinical trials of breast cancer patients treated with docetaxel and tamoxifen to evaluate COXEN predictions independently. Third, COXEN was used to carry out in silico screening of 45,545 compounds to identify new candidate agents that might be selectively active against bladder cancer cells in the BLA-40.

In the first application of the COXEN algorithm, for example, Cell Sets 1 and 2 were the NCI-60 and BLA-40 cell panels, respectively; the Step 1 drug activities were those assessed by DTP in the NCI-60 using a 48-hour sulforhodamine B assay; the “molecular characteristic” in Steps 2 and 4 was transcript expression level, as assessed using Affymetrix HG-U133A microarrays; the algorithm in Step 3 was “Significance Analysis of Microarrays (SAM)” or two-sample t-test; Step 5 was a novel “co-expression extrapolation” algorithm; Step 6 was another novel algorithm, “Misclassification-Penalized Posterior” (MiPP), which we recently introduced for selection of the best mathematical “models” for the prediction; and applying the prediction models obtained in Step 6, independent testing of the predictions on BLA-40 cells was performed, mimicking the way the assay for the NCI-60 by DTP.

One of ordinary skill in the art will appreciate that the algorithm step in 3 can be performed with other methods instead of SAM, or a two-sample t-test, or modifications thereof, which instead can be referred to a s “statistical identification of agent activity biomarkers of interest.”

Although it may not be intuitively obvious, steps 3 and 5 cannot be omitted; the algorithm uses, not the entire molecular signature, but those aspects of the signature that most strongly predict the drug's activity and that also reflect a pattern of co-expression between the two sets of cancer cells. As will be shown below, simply using the entire molecular signature (or even the entire drug activity molecular signature portion of it) does not work well.

Predicting drug activity in bladder cancer cells Applying the particular implementation of COXEN shown in FIG. 1A and described in detail in Methods, we used the NCI-60 data to predict drug activities in the BLA-40. We then tested the predictions independently for two drugs, cisplatin and paclitaxel, that are used clinically against bladder cancer. For that test, we focused first on the ten most sensitive and ten most resistant BLA-40 lines (top and bottom 25% of the BLA-40 drug responses, see Methods). As shown in Table 1B, prediction accuracies for the top three MiPP models averaged 85% (i.e., 90% of sensitive cells and 80% of resistant cells classified correctly) for cisplatin and 78% (83% of sensitive cells and 73% of resistant cells correct) for paclitaxel. As expected, those classification accuracies were lower than the ones obtained for the NCI-60 (Table 1A) but, nonetheless, highly statistically significant (two-tailed p-value=0.002 for cisplatin and 0.012-0.042 for the three models for paclitaxel). For cisplatin, nine sensitive cell lines (all except umuc9) and eight resistant cell lines (all except crl7197 and kk47) were consistently correctly classified by the three prediction models. For paclitaxel, one sensitive (X235jp) and one resistant (umuc1) cell line were consistently misclassified by the top three models

Since the a priori decision to classify sensitive and resistant cells was heuristic and did not provide predictive results for the “in-between” cell types, we next analyzed the quantitative relationship between COXEN-predicted and actual activity values for all 40. The results for the top MiPP model, shown in FIGS. 1B and 1C for cisplatin and paclitaxel, respectively, were highly significant (Spearman correlation coefficient p-value=0.016 for cisplatin and 0.006 for paclitaxel). Note that given non-comparability of the scales for MiPP score and log(GI50) values, we focused on the rank-based Spearman correlation.

The predictive power of the algorithm can be expressed more fully in a receiver-operator characteristics (ROC) analysis. As is often useful in biomarker studies, the ROC formulation permits free choice of a set-point to use in balancing the costs of false-positive and false-negative predictions. Non-parametric tests such as Wilcoxon rank-sum test can be calculated for comparing two different ROC curves. FIG. 1D contrasts the ROC curves obtained for cisplatin from the full COXEN algorithm with those obtained by leaving out either the drug chemosensitivity signature step (Step 3) or the co-occurrence step (Step 5). Clearly, the predictions were far superior when the entire algorithm was used. Importantly, no chemosensitivity data on the BLA-40 cells were used to “tune” any part of the COXEN algorithm to obtain the results for described here or elsewhere in the study.

The Clustered Image Maps (heat maps) in FIGS. 2B-C illustrates graphically the raison d'etre for the “co-occurrence” step (Step 5) in COXEN. Without that step (FIG. 2B), the cell types tend to sort themselves out according to whether they are NCI-60 or BLA-40; with that step (FIG. 2C), the cells of the two panels tend to intermingle and (as one would wish) to cluster according to their sensitivity to the drug. FIGS. 2D and 2E show similar results for paclitaxel. In all cases, the co-occurrence step makes the difference between clustering by cell panel and clustering by sensitivity to the drug.

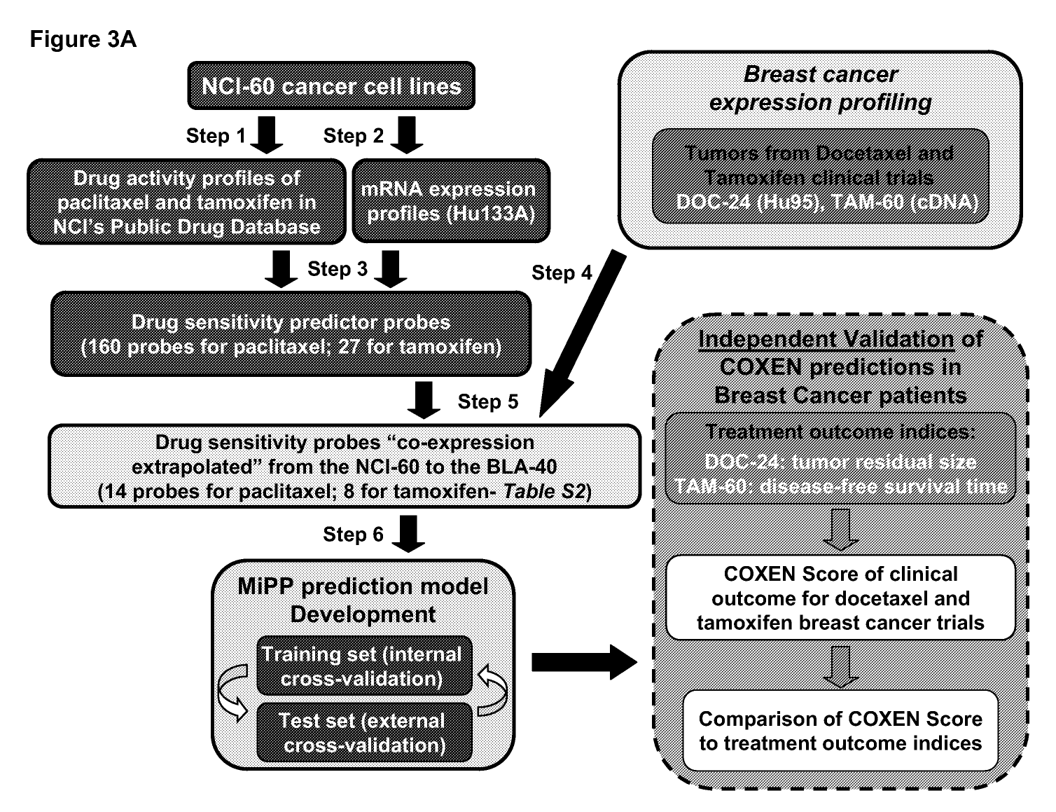

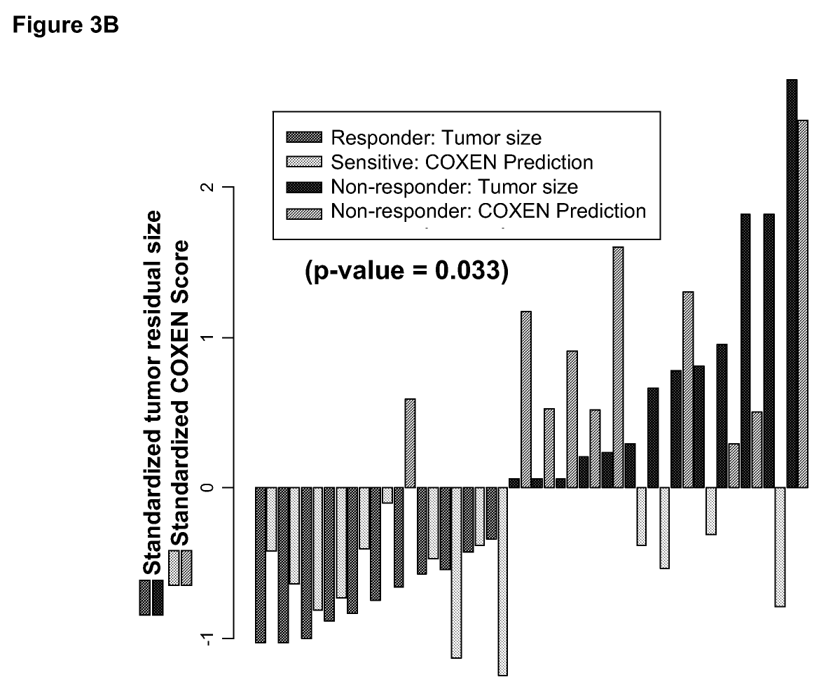

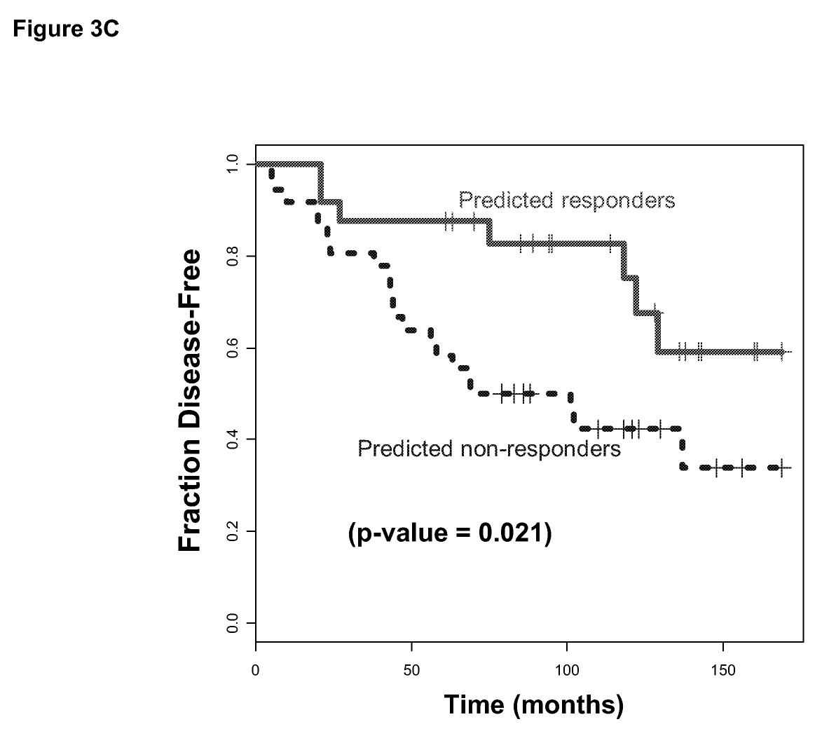

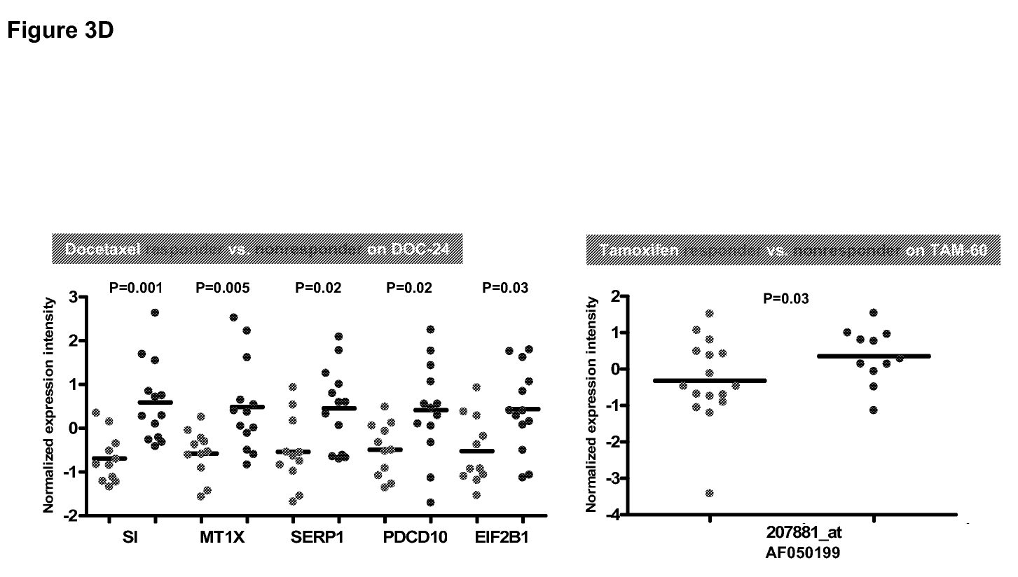

Prediction of clinical response to chemotherapeutics in human breast cancer patients Given the finding that COXEN could predict drug sensitivity, even in cell lines of histological types not included in the NCI-60 panel, we wondered whether an analogous algorithm would also have any predictive power for drug response in patients. Historically, it has proven difficult to predict drug activity in mouse xenografts from cell line data or clinical responses from mouse xenograft data. So, our hope and our hypothesis was that by eliminating the “middle-mouse,” we might be able to achieve some predictiveness for the clinic. Hence, we developed a modification of COXEN that aligns the NCI-60 gene expression data with expression data from patients' tumors, rather than cell lines. FIG. 3A shows the algorithm in schematic form. For test cases, we chose two cohort-based breast cancer clinical trials, DOC-24 (24 patients treated with docetaxel) and TAM-60 (60 patients treated with tamoxifen). Those trials satisfied several criteria for our analysis, most important among them: (1) the clinical response data were publicly available; (2) the patients' tumors had been transcript-profiled; (3) the treatment was single-agent, mirroring the single-agent treatments of the NCI-60 panel. The latter criterion was hardest to satisfy, since most clinical efficacy trials are on drug combinations.

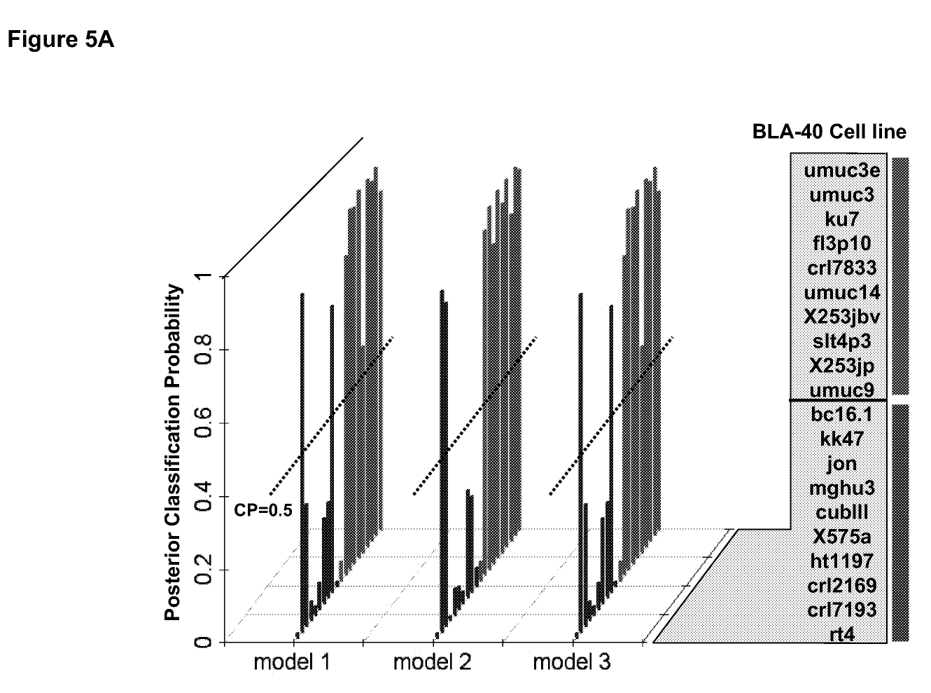

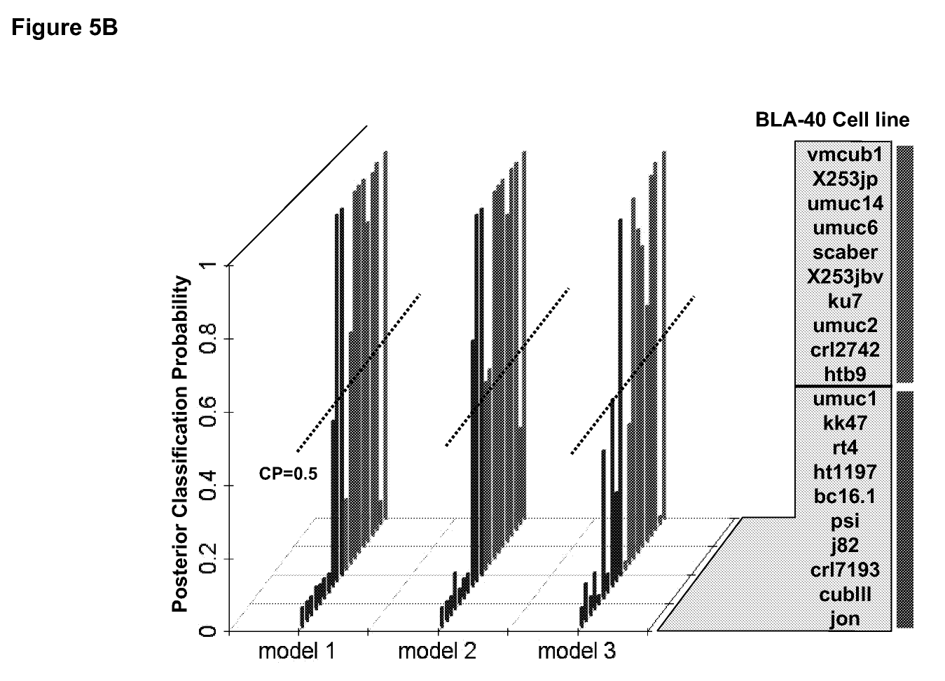

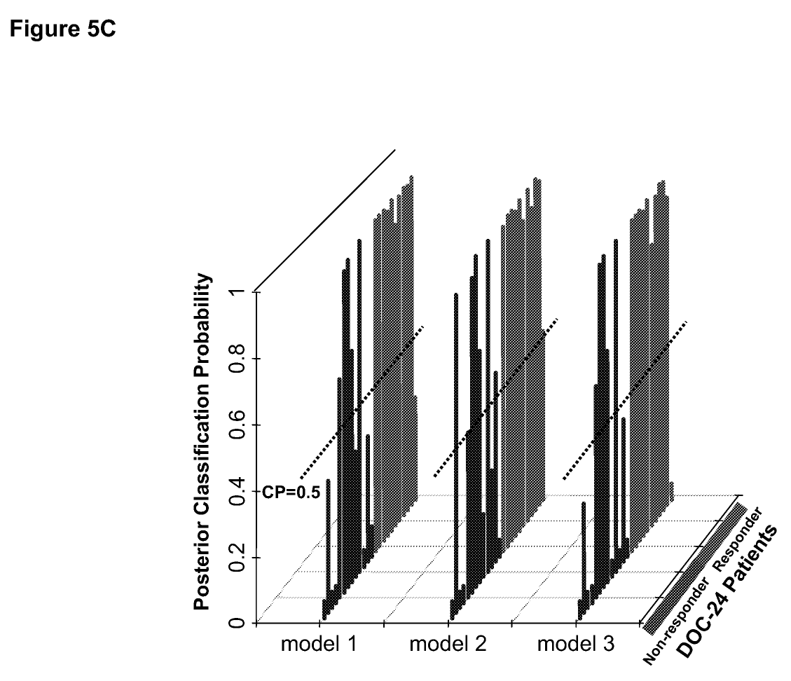

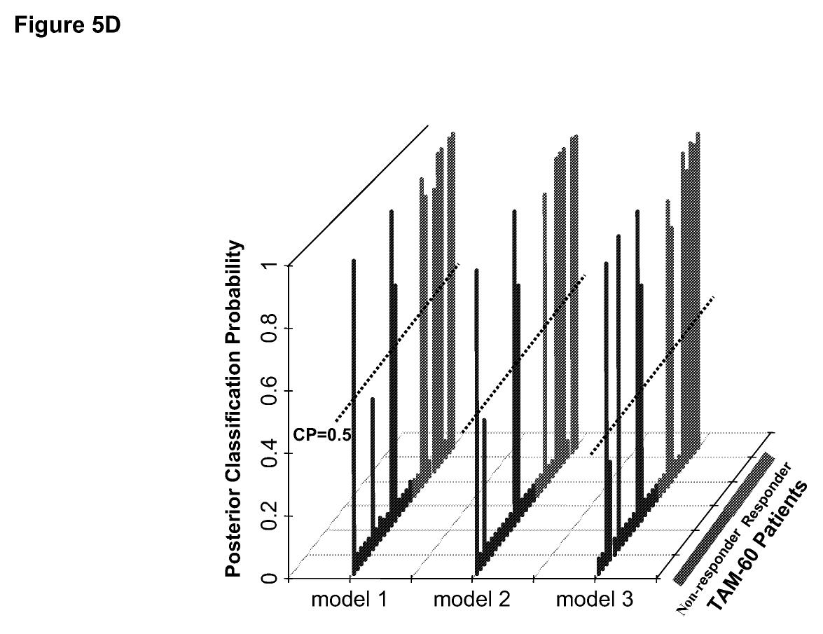

By analogy with our algorithm for bladder cancer cell lines, we first identified the drug signature genes with high degrees of co-expression between the NCI-60 and each of the clinical microarray data sets (i.e., those for the docetaxel and tamoxifen trials). We then derived the corresponding COXEN classification models based on the NCI-60 drug responses and microarray data. Predictions of response after four cycles of neoadjuvant chemotherapy with docetaxel (DOC-24) were evaluated for the 11 responder and 13 non-responder patients reported in the original study. As summarized in Table 2, the classification prediction accuracies across the top three MiPP models were uniformly 75%. The models also showed consistent prediction performance when assessed in terms of continuous variables (FIG. 5A-B for cisplatin and paclitaxel on the BLA-40, FIG. 5C for the docetaxel trial (DOC-24), and for FIG. 5D the tamoxifen trial (TAM-60)). As would be expected, the accuracy for clinical responses was lower than that for the bladder cancer cell lines, but nevertheless statistically significant (p-value=0.022). We next directly compared our MiPP predictive scores with the patients' residual tumor sizes after mathematical standardization (FIG. 3B). Given non-comparability of the scales for MiPP score and tumor size, we again used the rank-based Spearman correlation, which was significant (p-value=0.033).

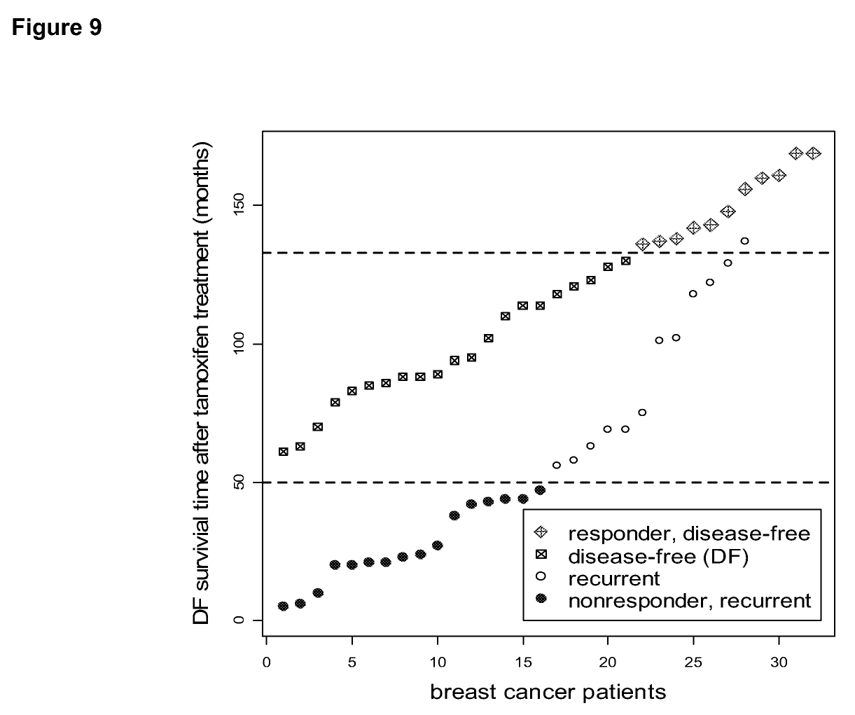

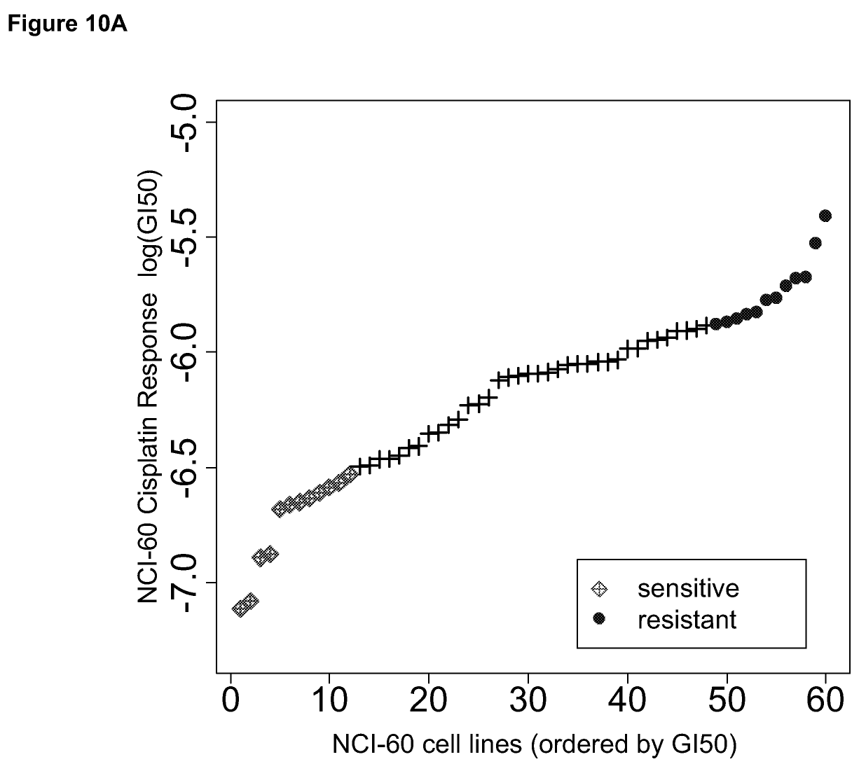

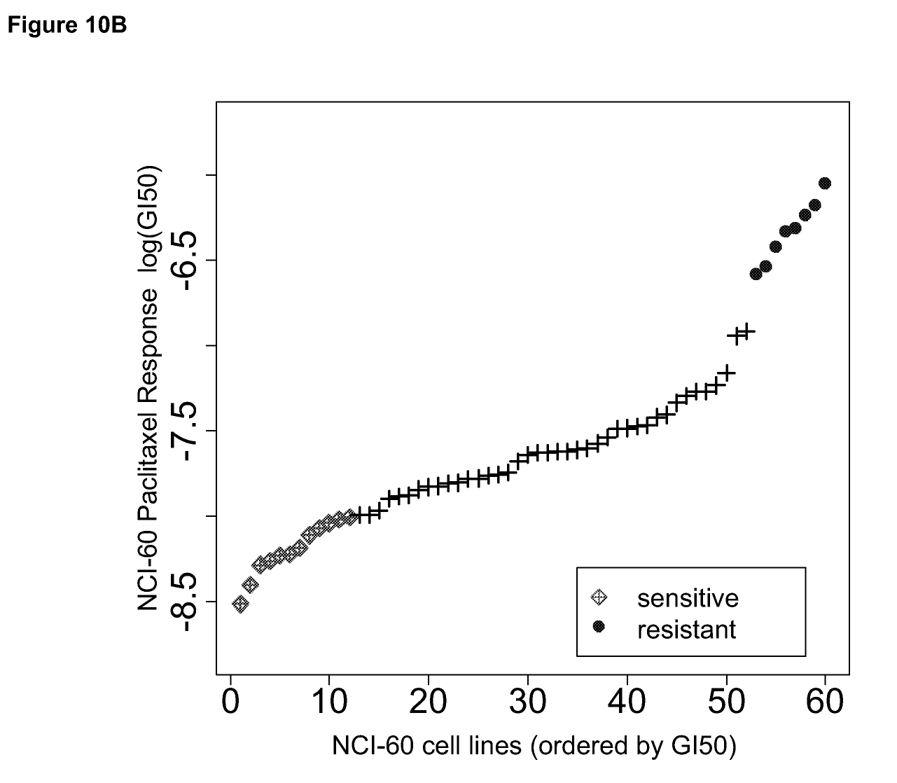

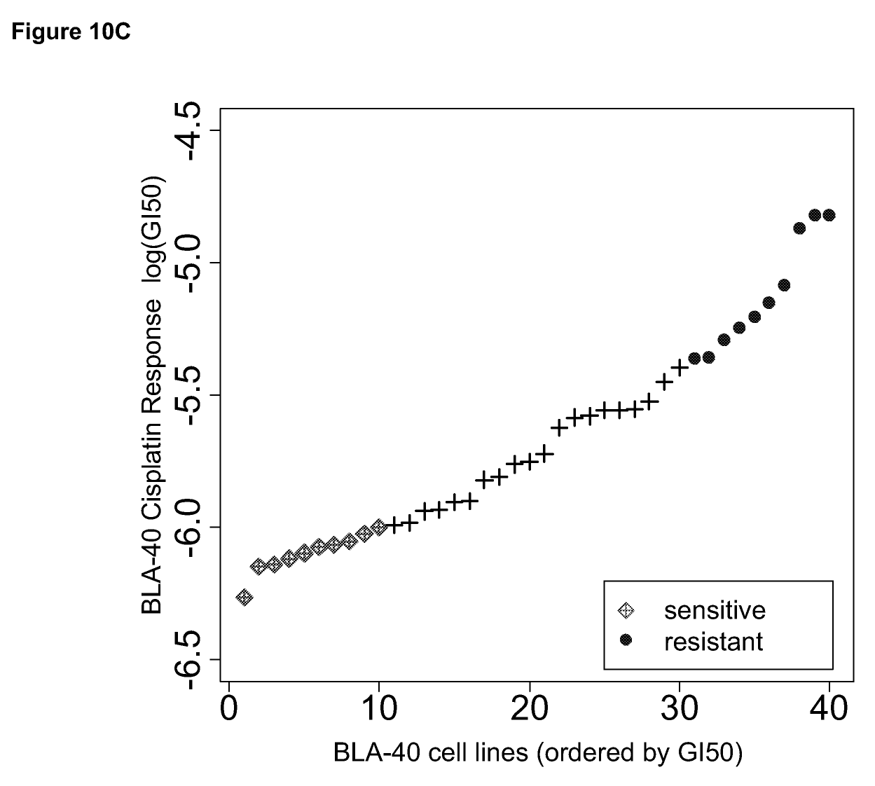

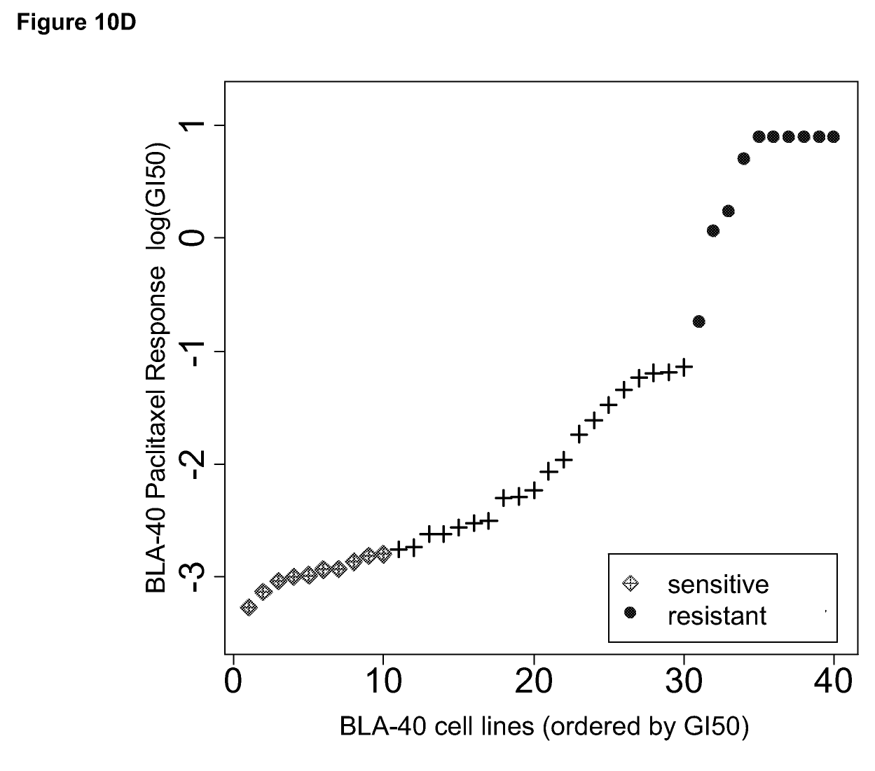

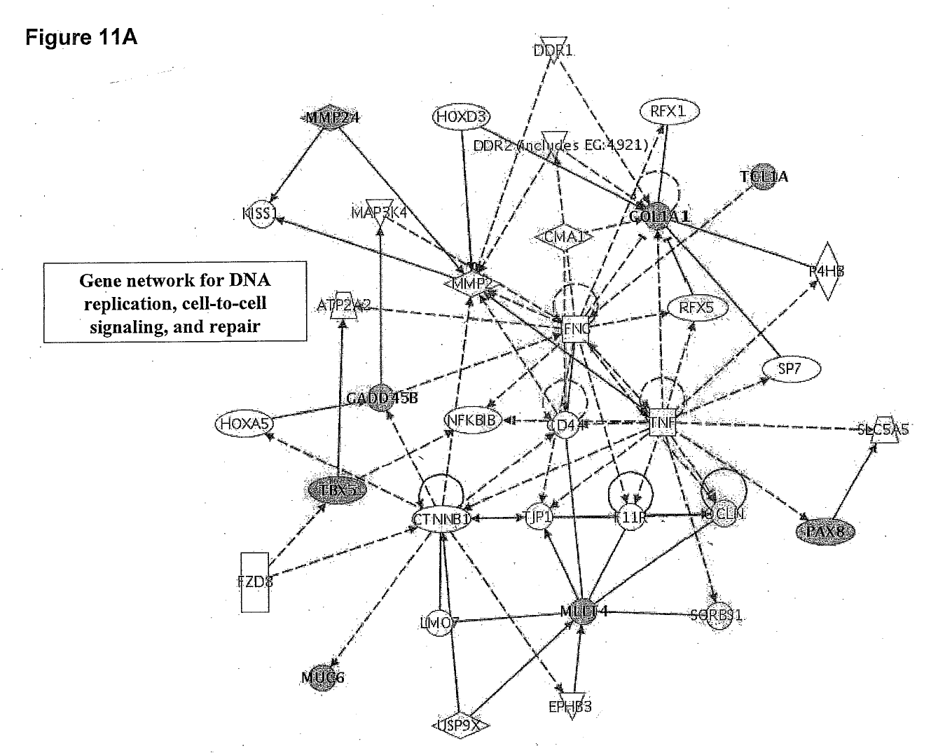

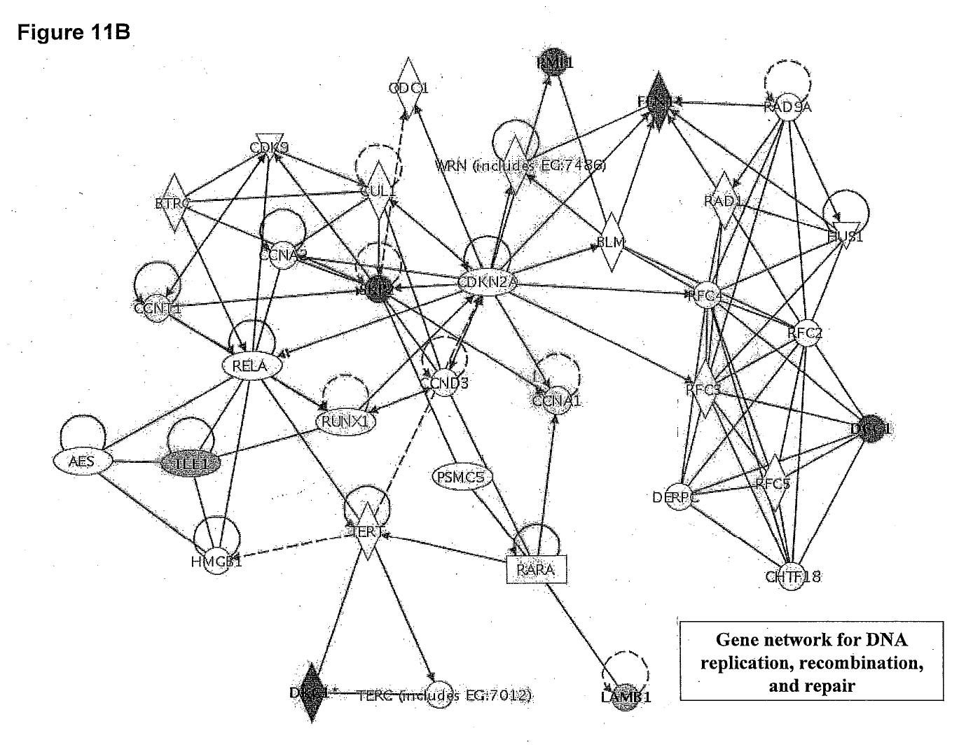

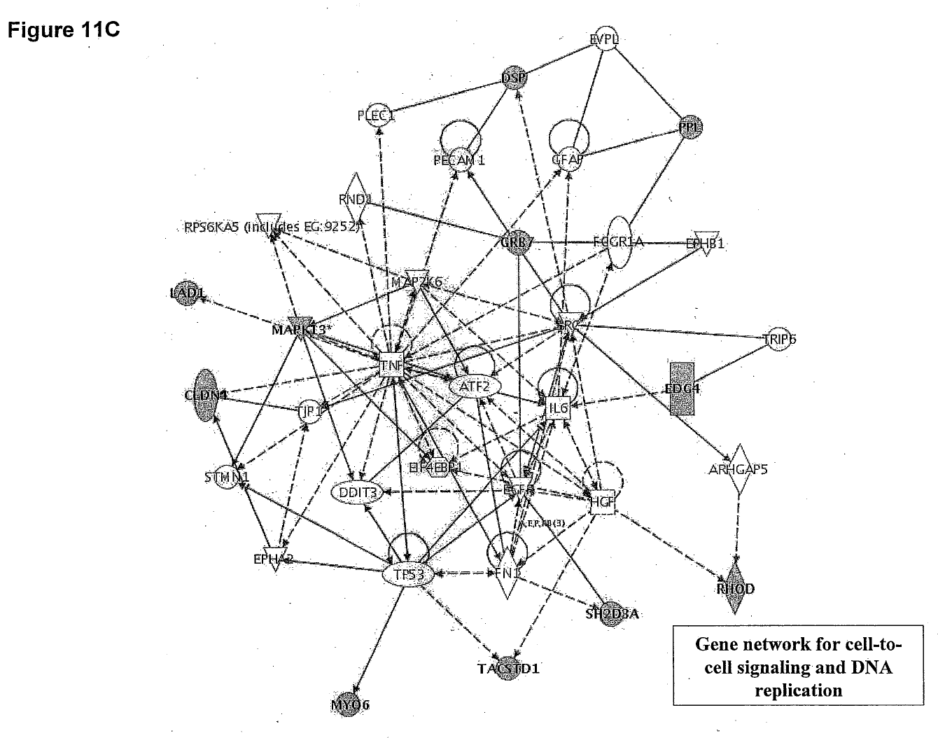

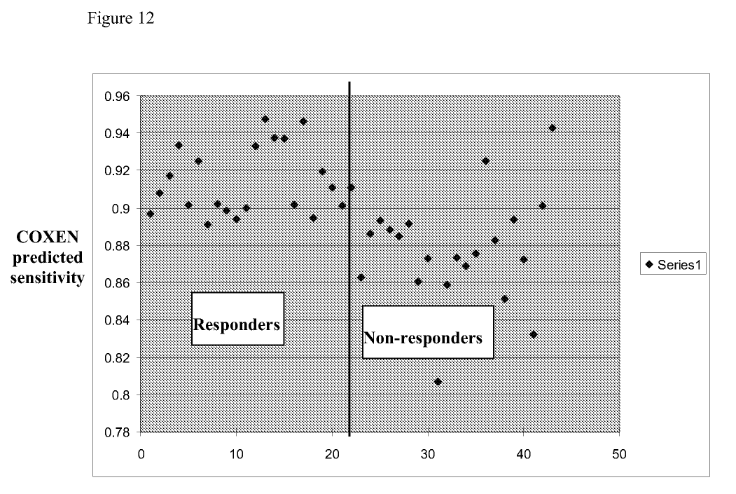

In the tamoxifen clinical trial (TAM-60), 60 postmenopausal breast cancer patients with estrogen receptor-positive tumors were treated and followed for up to 180 months. Genome-wide expression profiling was performed on the primary tumors using a customized cDNA microarray platform. The study data did not include measures of short-term tumor response but did include long-term disease-free survival and disease-recurrence times. Those data were difficult to relate directly to drug responses per se because such outcomes are likely to depend substantially on factors other than drug treatment. However, careful examination of the data indicated that patients could be classified into two distinct groups based on time to recurrence: those who recurred within a relatively short time (<50 months) after tamoxifen treatment and those who survived long-term (>130 months). Hence, we made the assumption that early-recurrence patients constituted tamoxifen non-responders and long-term survivors constituted responders (FIG. 9). From those observations, we identified 11 responders and 16 non-responders prior to, and independent of, making the COXEN predictions. Note that one would expect less, rather than more, predictive power from the algorithm insofar as factors other than response to tamoxifen confounded the classification as responders or non-responders.

The prediction accuracies across the top three MiPP prediction models averaged 71% (p-values 0.019—0.052) for responders and non-responders in the tamoxifen trial (Table 2). To examine the robustness of COXEN predictions in all 60 patients, we examined the Kaplan-Meier survival curves. In that analysis, the predicted responder group based on the top MiPP prediction model showed a significantly longer disease-free survival time (FIG. 3C) than the predicted non-responder group (p-value=0.021) 13. Overall, the prediction performance can be considered impressive given that 1) only a small proportion (about 11%) of probe sets were matched in their annotation between the Affymetrix HG-U133A and customized cDNA microarray data, and 2) we used the surrogate of disease-free survival time instead of a more conventional outcome measure (such as complete or partial remission), which would probably have related more closely to the in vitro chemosensitivity data. Finally, as for the bladder studies above, it is important to note that validations were done prospectively, without any “tuning” of the model on the basis of response data from the clinical trials.

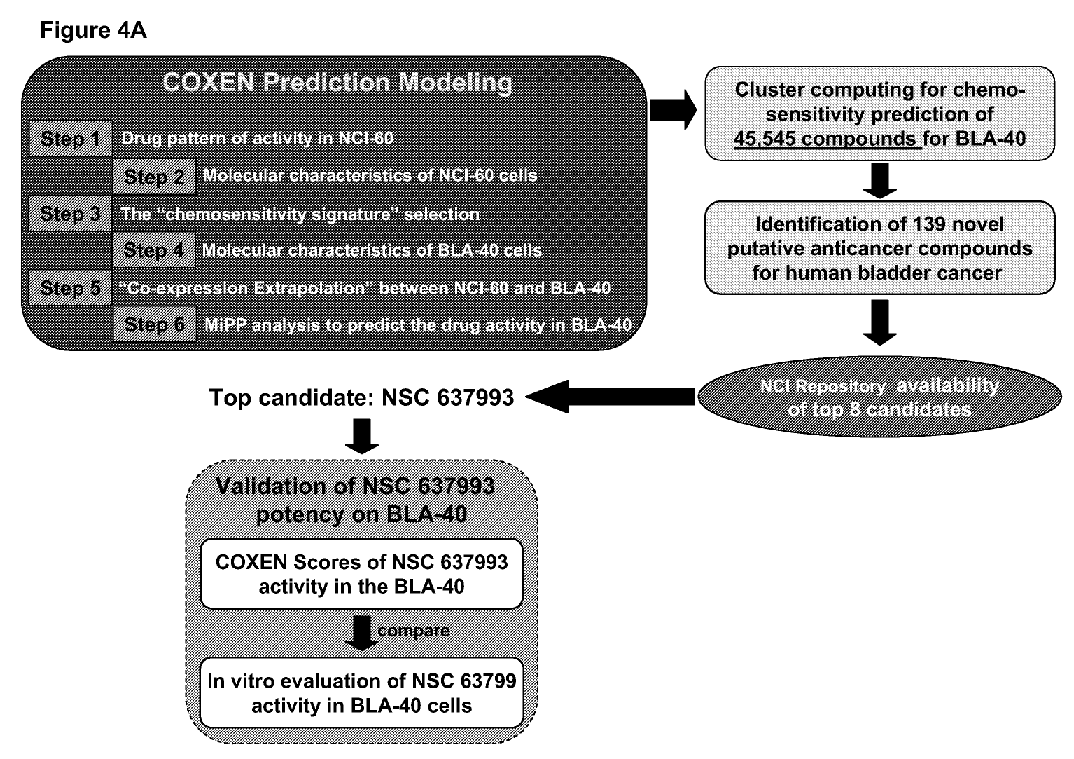

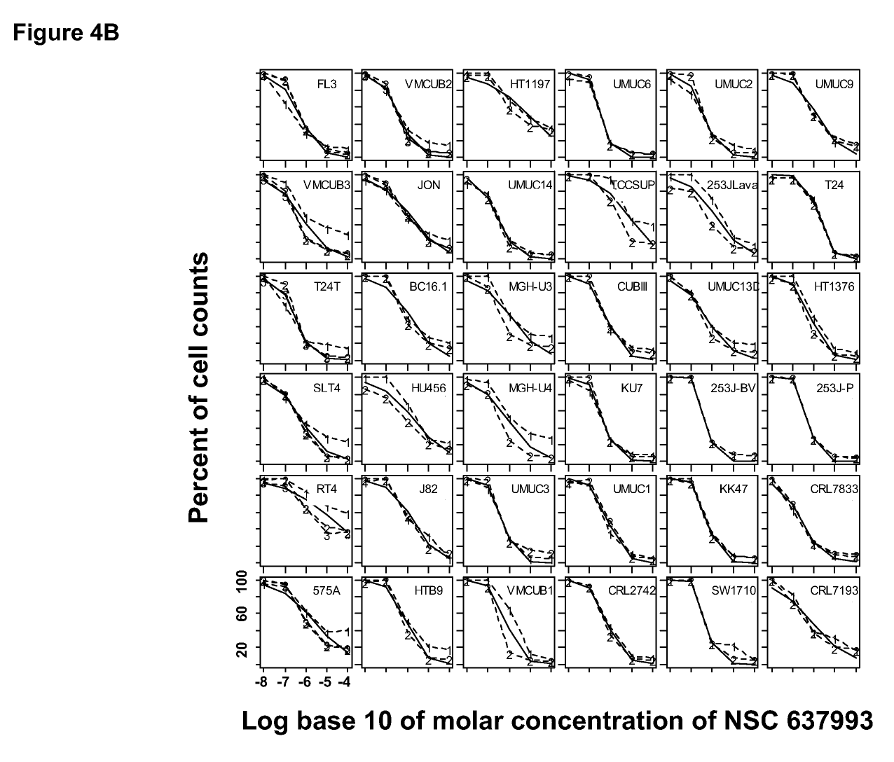

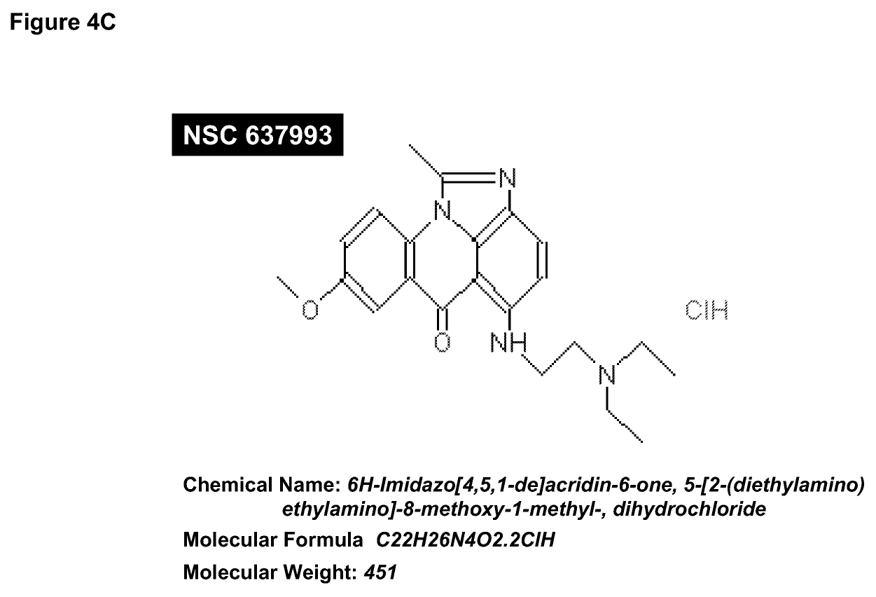

Use of COXEN for computational drug discovery Given the encouraging predictive performance of COXEN, both in vitro (for BLA-40 bladder cancer lines) and in patients (with breast cancer), we applied it in a novel way to drug discovery shown schematically in FIG. 4A. For each of the 45,545 compounds with data publically available from the DTP, we used COXEN to predict in silico chemosensitivity patterns for cells in the BLA-40 panel. The calculations for so many compounds were computer-intensive, taking 54 days (24 hrs/day) on a 32-node computer cluster at the University of Virginia. For prediction of each drug's activity in the BLA-40, we averaged the classification probabilities of the top five MiPP models identified.

In an initial screen we identified 139 compounds for which COXEN predicted 50% growth inhibitory concentrations (GI50's) for at least 35% of the BLA-40 cells. For eight of those compounds, >50% of the BLA-40 were predicted to have submicromolar GI50's. Not all of the candidate compounds were available from the DTP but, fortunately, our top hit, NSC637993 was, and we were able to assay it for growth inhibition in the BLA-40 panel. The measured GI50 values were less than 10-6M for >60% of the cell types, consistent with prediction 61.8% (FIG. 4B). Most notably, NSC637993 was more potent overall in the BLA-40 bladder cancers than in any of the organ-of-origin types included in the NCI-60 (data not shown). It was even more potent in the BLA-40 than in the NCI-60 leukemias, which are generally the most sensitive cells.

TABLE 1
Top MIPP classification models on chemosensitivity response
prediction on sensitive and resistant cell lines for cisplatin and paclitaxel.
A) Top three MiPP models and their independent-set validated
prediction performance on the NCI-60, B) Predicted and actual
performance of the models shown in (A) in the BLA-40 panel.
Table 1A

| | Predictor gene models | Mean | Prediction Accuracy |
|---|---|---|---|
| | composition | Error Rate | Mean (95% CI) |
| Cisplatin | | | |
| Model 1 | EDG4, RHOD, MYO6 | 0.044 | 0.96 (0.89, 1.00) |
| Model 2 | RHOD, MYO6 | 0.039 | 0.96 (0.88, 1.00) |
| Model 3 | DSP, RHOD, MYO6 | 0.054 | 0.95 (0.75, 0.99) |
| Paclitaxel | | | |
| Model 1 | DCC1, TLE1, KIAA0947 | 0.068 | 0.93 (0.83, 0.99) |
| Model 2 | DKC1 (201478), TLE1, | 0.046 | 0.95 (0.83, 1.00) |
| | KIAA0947, DCC1 | | |
| Model 3 | DKC1 (201479), DCC1, | 0.045 | 0.94 (0.84, 1.00) |
| | TLE1, KIAA0947 | | |

Table 1B

| | Sensitive* | Resistant* | Overall | Overall |
|---|---|---|---|---|
| | N = 10 | N = 10 | N = 20 | (p-value**) |
| Cisplatin | | | | |
| Model 1 | 9/10 | 8/10 | 85% (17/20) | 0.002 |
| Model 2 | 9/10 | 8/10 | 85% (17/20) | 0.002 |
| Model 3 | 9/10 | 8/10 | 85% (17/20) | 0.002 |
| Paclitaxel | | | | |
| Model 1 | 8/10 | 8/10 | 80% (16/20) | 0.012 |
| Model 2 | 9/10 | 7/10 | 80% (16/20) | 0.012 |
| Model 3 | 8/10 | 7/10 | 75% (15/20) | 0.041 |

**Derived by a binomial test from a null hypothesis that prediction is random
^{#}Classification of cell lines as sensitive and resistant is based on their posterior classification probabilities from each model.

TABLE 2
Evaluation of predictive performance of top three MIPP
classification models on chemotherapeutic response of the
breast cancer patients in the docetaxel (DOC-24) and
tamoxifen trials (TAM-60).

| | Responder* | Nonresponder* | Overall | Overall |
|---|---|---|---|---|
| Docetaxel | N = 11 | N = 13 | N = 24⁺ | (p-value**) |
| Model 1 | 10/11 | 8/13 | 75% (18/24) | 0.022 |
| Model 2 | 11/11 | 7/13 | 75% (18/24) | 0.022 |
| Model 3 | 10/11 | 8/13 | 75% (18/24) | 0.022 |
| | Responder\hat{ } | Nonresponder\hat{ } | Overall | Overall |
| Tamoxifen | N = 11 | N = 16 | N = 27 | (p-value**) |
| Model 1 | 7/11 | 13/16 | 74% (20/27) | 0.019 |
| Model 2 | 6/11 | 13/16 | 70% (19/27) | 0.052 |
| Model 3 | 7/11 | 12/16 | 70% (19/27) | 0.052 |

⁺correctly classified according to outcome reported in the original study¹¹
\hat{ }correctly classified according to criteria shown in FIG. 9 and described in results.
**Derived by a binomial test from a null hypothesis that such a prediction is random.
^{#}Classification of patients as responders and nonresponders is based on their posterior classification probabilities (CP) from each model, i.e., responder if CP > 0.5 and nonresponder if CP < 0.5.

Microarray Gene Expression Data on breast cancer patient populations HG-U133A GeneChip® arrays from two recent breast cancer studies (Two validation/prediction sets with 49 and 251 patients; BRE-49 and BRE-251) were used for our novel drug discovery (Farmer et al., Oncogene 24, 4660-71, 2005; Miller et al., Proc Natl Acad Sci USA 102, 13550-5, 2005). When quality control checks passed, Affymetrix GeneChip® array files of the NCI-60 and breast cancer patients were analyzed with the RMA analysis software to obtain the expression intensity values of the microarray data. The identified compounds relevant to breast cancer in particular are provided in Table 3.

Novel anticancer drug discovery for bladder cancer: The Bladder cancer drug discovery was performed using BLA-40 and our internal microarray data set of 85 human bladder cancer patients (BLA-85) (Two validation/prediction sets; Table 4).

Novel anticancer drug discovery for Prostate cancer: The prostate cancer drug discovery was performed using the data set of 88 patient samples (Table 5). Yu et al., Gene expression alterations in prostate cancer predicting tumor aggression and preceding development of malignancy, J. Clin. Oncol. 22, 2004, 2790-2799.

Novel anticancer drug discovery for melanoma: The Melanoma cancer drug discovery was performed using the data set of 70 patients (Table 6). Talantov D, Mazumder A, Yu J X, Briggs T et al. Novel genes associated with malignant melanoma but not benign melanocytic lesions. Clin Cancer Res 2005 Oct. 15; 11(20):7234-42.

Novel anticancer drug discovery for Pancreatic cancer: The pancreatic cancer drug discovery was performed using the data set of 49 patients (Table 7). Ishikawa M, Yoshida K, Yamashita Y. Ota J et al. Experimental trial for diagnosis of pancreatic ductal carcinoma based on gene expression profiles of pancreatic ductal cells. Cancer Sci 2005 July; 96(7):387-93.

TABLE 3
Compounds Identified Relevant to Breast Cancer Treatment

| | BRE-49 | BRE-251 | | Clinical |
|---|---|---|---|---|
| | predicted | predicted | Mean predicted | response |
| NSC # | response rate | response rate | response rate | rate |
| 715114 | 58.8 | 55.9 | 56.3 | |
| 710904 | 54.3 | 51.2 | 51.7 | |
| 691895 | 60.2 | 49.9 | 51.6 | |
| 170105 | 49.8 | 49.6 | 49.6 | |
| 693539 | 54.3 | 47.8 | 48.9 | |
| 607281 | 51 | 46.9 | 47.6 | |
| 682996 | 42.4 | 48.6 | 47.6 | |
| 125066 | 51.4 | 46.4 | 47.2 | |
| 643813 | 46.5 | 47.1 | 47 | |
| 19893 | 46.1 | 47 | 46.9 | 44.4 |
| 707691 | 46.9 | 46.8 | 46.8 | |
| 357777 | 47.3 | 46.5 | 46.7 | |
| 200692 | 42.4 | 45.5 | 45 | |
| 701109 | 45.7 | 44.9 | 45 | |
| 620124 | 46.9 | 43.2 | 43.8 | |
| 706233 | 39.6 | 44.4 | 43.6 | |
| 49689 | 44.1 | 43.4 | 43.5 | |
| 683140 | 44.1 | 43.4 | 43.5 | |
| 205628 | 41.6 | 42.6 | 42.5 | |
| 669793 | 35.9 | 43.7 | 42.4 | |
| 711737 | 41.2 | 42.5 | 42.3 | |
| 657028 | 44.1 | 41.7 | 42.1 | |
| 710548 | 46.9 | 41 | 41.9 | |
| 708424 | 44.9 | 40.9 | 41.5 | |
| 711022 | 48.2 | 40.2 | 41.5 | |
| 654376 | 36.7 | 41.8 | 40.9 | |
| 720704 | 43.3 | 40.2 | 40.7 | |
| 667932 | 37.6 | 41.2 | 40.6 | |
| 683648 | 40.4 | 40.3 | 40.3 | |
| 682860 | 43.3 | 39.4 | 40.1 | |
| 642061 | 44.5 | 39.1 | 40 | |
| 709361 | 38.8 | 40.2 | 40 | |
| 673190 | 42 | 39.3 | 39.7 | |
| 674493 | 39.2 | 39.8 | 39.7 | |
| 226080 | 39.2 | 39.4 | 39.4 | |
| 143095 | 38.8 | 39.4 | 39.3 | |
| 655978 | 38 | 39.5 | 39.3 | |
| 1012 | 38.8 | 39 | 38.9 | |
| 127716 | 37.1 | 39.2 | 38.9 | |
| 673844 | 42.4 | 38.1 | 38.8 | |
| 703107 | 38.8 | 38.6 | 38.7 | |
| 707083 | 40 | 38.3 | 38.6 | |
| 625987 | 40 | 38.2 | 38.5 | |
| 633713 | 40 | 38.2 | 38.5 | |
| 698791 | 42.4 | 37.7 | 38.5 | |
| 645830 | 42 | 37.7 | 38.4 | |
| 268993 | 40.8 | 37.6 | 38.1 | |
| 652886 | 36.7 | 38.3 | 38.1 | |
| 667872 | 40.4 | 37.4 | 37.9 | |
| 666227 | 39.6 | 37.2 | 37.6 | |
| 351078 | 40 | 37.1 | 37.5 | |
| 168516 | 33.5 | 38 | 37.3 | |
| 665948 | 42 | 36.3 | 37.3 | |
| 674316 | 37.6 | 36.1 | 36.3 | |
| 18268 | 38.8 | 35.8 | 36.3 | |
| 666038 | 38.8 | 35.8 | 36.3 | |
| 710204 | 39.2 | 35.6 | 36.2 | |
| 640967 | 33.1 | 36.6 | 36 | |
| 690021 | 28.6 | 37.5 | 36 | |
| 702030 | 36.7 | 35.8 | 35.9 | |
| 712206 | 40 | 35.1 | 35.9 | |
| 718020 | 38 | 35.5 | 35.9 | |
| 650565 | 39.6 | 34.9 | 35.7 | |
| 720379 | 37.1 | 35.1 | 35.5 | |
| 693544 | 35.9 | 35.1 | 35.3 | |
| 717463 | 35.5 | 35.2 | 35.3 | |
| 321803 | 35.5 | 35.1 | 35.2 | |
| 337591 | 37.1 | 34.7 | 35.1 | |
| 194350 | 39.2 | 34.2 | 35 | |
| 677734 | 36.3 | 34.7 | 35 | |
| 299879 | 42.4 | 33.5 | 34.9 | |
| 714604 | 34.3 | 35.1 | 34.9 | |
| 678007 | 37.1 | 34.3 | 34.7 | |
| 657561 | 40 | 33.3 | 34.4 | |
| 715227 | 34.7 | 34.3 | 34.4 | |
| 710557 | 38 | 33.5 | 34.3 | |
| 715147 | 38 | 33.5 | 34.3 | |
| 338304 | 31.4 | 34.7 | 34.1 | |
| 671168 | 34.7 | 34 | 34.1 | |
| 359463 | 38 | 32.9 | 33.7 | |
| 670876 | 37.6 | 33 | 33.7 | |
| 671886 | 35.9 | 33.3 | 33.7 | |
| 693119 | 33.5 | 33.8 | 33.7 | |
| 227279 | 28.2 | 34.6 | 33.5 | |
| 657346 | 40 | 32.3 | 33.5 | |
| 624851 | 36.7 | 32.8 | 33.5 | |
| 625543 | 33.5 | 33.5 | 33.5 | |
| 668296 | 36.3 | 32.9 | 33.5 | |
| 697653 | 39.2 | 32.4 | 33.5 | |
| 698685 | 38.8 | 32.4 | 33.5 | |
| 146268 | 32.7 | 33.2 | 33.1 | |
| 702322 | 34.3 | 32.8 | 33.1 | |
| 150014 | 30.6 | 33.5 | 33 | |
| 659609 | 31 | 33.4 | 33 | |
| 703136 | 33.7 | 32.9 | 33 | |
| 715599 | 31 | 33.3 | 32.9 | |
| 137049 | 33.9 | 32.4 | 32.7 | |
| 622114 | 34.7 | 32.3 | 32.7 | |
| 708423 | 36.7 | 31.9 | 32.7 | |
| 658886 | 33.9 | 32.4 | 32.6 | |
| 668301 | 31.4 | 32.8 | 32.6 | |
| 698959 | 35.5 | 32 | 32.6 | |
| 645205 | 35.9 | 31.9 | 32.5 | |
| 376266 | 32.2 | 32.5 | 32.5 | |
| 676944 | 36.3 | 31.4 | 32.2 | |
| 666388 | 35.1 | 31.6 | 32.1 | |
| 322355 | 33.9 | 31.7 | 32.1 | |
| 349051 | 33.1 | 31.8 | 32 | |
| 681279 | 33.9 | 31.6 | 32 | |
| 628562 | 31.8 | 31.9 | 31.9 | |
| 638736 | 32.2 | 31.8 | 31.9 | |
| 661580 | 33.1 | 31.6 | 31.8 | |
| 667886 | 38.4 | 30.5 | 31.8 | |
| 689529 | 30.6 | 32 | 31.8 | |
| 715669 | 31.8 | 31.8 | 31.8 | |
| 678156 | 34.7 | 30.9 | 31.5 | |
| 698177 | 31.8 | 31.4 | 31.5 | |
| 674996 | 31.4 | 31.4 | 31.4 | |
| 679024 | 31 | 31.5 | 31.4 | |
| 708390 | 33.9 | 30.9 | 31.4 | |
| 698087 | 26.5 | 32.2 | 31.3 | |
| 708387 | 35.1 | 30.5 | 31.3 | |
| 665089 | 34.7 | 30.4 | 31.1 | |
| 717519 | 31.4 | 30.9 | 31 | |
| 657025 | 40 | 29.2 | 30.9 | |
| 709137 | 31 | 30.8 | 30.9 | |
| 716871 | 34.7 | 30.1 | 30.9 | |
| 701380 | 31.4 | 30.7 | 30.8 | |
| 670694 | 35.5 | 29.8 | 30.7 | |
| 688220 | 34.3 | 30 | 30.7 | |
| 372944 | 32.2 | 30.3 | 30.6 | |
| 614554 | 38.4 | 29 | 30.5 | |
| 123127 | 35.5 | 29.6 | 30.5 | |
| 681454 | 29.4 | 30.7 | 30.5 | |
| 637399 | 30.6 | 30.4 | 30.4 | |
| 694950 | 31 | 30.2 | 30.3 | |
| 720553 | 31 | 30.2 | 30.3 | |
| 662193 | 38 | 28.6 | 30.1 | |
| 677949 | 27.3 | 30.7 | 30.1 | |
| 683367 | 26.9 | 30.7 | 30.1 | |
| 693563 | 24.5 | 31.2 | 30.1 | |
| 305819 | 31 | 29.8 | 30 | |
| 354670 | 32.2 | 29.3 | 29.8 | |
| 644751 | 32.7 | 29.2 | 29.7 | |
| 682991 | 26.5 | 30.4 | 29.7 | |
| 690268 | 34.7 | 28.6 | 29.6 | |
| 710342 | 31.4 | 29.2 | 29.6 | |
| 639521 | 35.1 | 28.3 | 29.4 | |
| 682765 | 27.8 | 29.7 | 29.4 | |
| 717473 | 30.2 | 29.2 | 29.4 | |
| 666123 | 29.8 | 29.2 | 29.3 | |
| 58575 | 35.5 | 28 | 29.3 | |
| 5550 | 33.9 | 28.3 | 29.2 | |
| 659998 | 37.1 | 27.5 | 29.1 | |
| 684836 | 33.1 | 28.2 | 29 | |

Compounds identified relevant to bladder cancer As discussed above, 139 compounds were identified using the methods of the invention which have particular relevance to bladder cancer. The compounds are summarized in Table 4.

TABLE 4
Compounds Identified Relevant to Bladder Cancer on BLA-85

| NSC | Clinical response | CP > 95% | CP > 50% |
|---|---|---|---|
| 637993 | | 61.6 | 65.5 |
| 713368 | | 56.1 | 68.9 |
| 676857 | | 56.1 | 67.1 |
| 676830 | | 51.8 | 60.5 |
| 128687 | | 51.8 | 65 |
| 645665 | | 50.8 | 54.7 |
| 679001 | | 50 | 70.8 |
| 676522 | | 50 | 57.9 |
| 382050 | | 48.9 | 56.8 |
| 676536 | | 48.4 | 58.9 |
| 236580 | | 48.4 | 61.6 |
| 634568 | | 48.2 | 66.2 |
| 682825 | | 48.2 | 64.7 |
| 678991 | | 47.9 | 59.5 |
| 740 | 30.6 | 47.8 | 57.5 |
| 699753 | | 47.6 | 65.3 |
| 172614 | | 47.6 | 56.6 |
| 19893 | 23.1 | 47.4 | 55.8 |
| 702396 | | 47.4 | 60.3 |
| 19893 | | 47.4 | 55.8 |
| 633713 | | 46.8 | 58.2 |
| 77830 | | 46.3 | 53.9 |
| 606699 | | 46.3 | 56.6 |
| 695939 | | 46.1 | 53.2 |
| 48300 | | 46.1 | 56.6 |
| 642492 | | 45.8 | 57.4 |
| 639831 | | 45.8 | 53.7 |
| 662373 | | 45.5 | 59.5 |
| 715559 | | 45.3 | 62.1 |
| 698147 | | 45.3 | 61.8 |
| 683257 | | 45.3 | 63.7 |
| 685106 | | 45 | 56.8 |
| 676832 | | 45 | 54.7 |
| 37364 | | 45 | 54.7 |
| 710560 | | 44.7 | 65 |
| 665364 | | 44.7 | 52.6 |
| 666787 | | 44.5 | 57.6 |
| 687523 | | 44.2 | 55.5 |
| 132483 | | 43.9 | 58.7 |
| 682817 | | 43.4 | 49.7 |
| 668525 | | 43.4 | 57.1 |
| 693120 | | 43.2 | 53.4 |
| 666110 | | 42.9 | 55.3 |
| 655751 | | 42.9 | 56.3 |
| 607281 | | 42.9 | 52.1 |
| 696860 | | 42.4 | 51.3 |
| 684902 | | 42.4 | 50.5 |
| 716954 | | 42.1 | 55.8 |
| 704172 | | 42.1 | 50 |
| 699756 | | 42.1 | 56.6 |
| 671902 | | 42.1 | 55 |
| 355063 | | 42.1 | 57.4 |
| 138333 | | 42.1 | 78.3 |
| 707691 | | 41.8 | 46.6 |
| 698791 | | 41.6 | 65 |
| 143095 | | 41.6 | 52.1 |
| 689138 | | 41.3 | 52.6 |
| 638304 | | 41.3 | 48.2 |
| 146268 | | 41.3 | 48.7 |
| 708496 | | 41.1 | 61.1 |
| 701373 | | 41.1 | 57.4 |
| 674130 | | 40.8 | 64.5 |
| 625502 | | 40.5 | 49.7 |
| 633258 | | 40.5 | 43.1 |
| 720135 | | 40.3 | 47.6 |
| 708387 | | 40.3 | 51.1 |
| 683922 | | 40.3 | 50.3 |
| 682991 | | 40.3 | 48.4 |
| 679024 | | 40.3 | 49.2 |
| 710557 | | 40 | 50 |
| 702435 | | 40 | 62.1 |
| 194617 | | 40 | 46.6 |
| 681632 | | 39.7 | 50.8 |
| 638498 | | 39.7 | 46.6 |
| 722308 | | 39.5 | 49.2 |
| 703126 | | 39.5 | 47.4 |
| 676944 | | 39.5 | 44.2 |
| 675223 | | 39.5 | 47.9 |
| 661580 | | 39.5 | 68.4 |
| 122301 | | 39.5 | 44.7 |
| 7365 | | 39.2 | 51.6 |
| 645392 | | 39.2 | 44.2 |
| 194350 | | 39.2 | 48.2 |
| 696923 | | 38.9 | 47.9 |
| 674233 | | 38.9 | 50 |
| 114341 | | 38.9 | 65.8 |
| 655901 | | 38.8 | 55.9 |
| 755 | | 38.7 | 57.6 |
| 680342 | | 38.7 | 53.7 |
| 667545 | | 38.7 | 51.3 |
| 666038 | | 38.7 | 50.3 |
| 302325 | | 38.7 | 48.2 |
| 643833 | | 38.5 | 52 |
| 703101 | | 38.4 | 49.5 |
| 701189 | | 38.4 | 45 |
| 698181 | | 38.2 | 53.4 |
| 696864 | | 37.9 | 48.9 |
| 690441 | | 37.9 | 59.2 |
| 651838 | | 37.6 | 45.5 |
| 372944 | | 37.6 | 46.6 |
| 710556 | | 37.4 | 51.3 |
| 666294 | | 37.4 | 48.9 |
| 347512 | | 37.3 | 52.2 |
| 720704 | | 37.2 | 52 |
| 698960 | | 37.1 | 51.8 |
| 687304 | | 37.1 | 48.9 |
| 685887 | | 37.1 | 42.9 |
| 636092 | | 37.1 | 52.4 |
| 606499 | | 37.1 | 51.6 |
| 35949 | | 37.1 | 42.6 |
| 717571 | | 36.8 | 58.9 |
| 706192 | | 36.8 | 52.6 |
| 696560 | | 36.8 | 48.7 |
| 638410 | | 36.8 | 49.2 |
| 382035 | | 36.8 | 49.2 |
| 1895 | | 36.8 | 46.1 |
| 680733 | | 36.6 | 51.3 |
| 658867 | | 36.6 | 47.1 |
| 618093 | | 36.6 | 60.5 |
| 71669 | | 36.5 | 53.3 |
| 667886 | | 36.3 | 48.2 |
| 59270 | | 36.3 | 50.8 |
| 382034 | | 36.3 | 45 |
| 329680 | | 36.2 | 59.2 |
| 640556 | | 36.1 | 53.7 |
| 639187 | | 36.1 | 53.9 |
| 637921 | | 36.1 | 45.3 |
| 676189 | | 35.8 | 42.4 |
| 671379 | | 35.8 | 47.1 |
| 665489 | | 35.8 | 57.4 |
| 703462 | | 35.5 | 45.5 |
| 684481 | | 35.5 | 55.3 |
| 366140 | | 35.5 | 45.8 |
| 153353 | | 35.5 | 49.5 |
| 10010 | | 35.5 | 54.5 |
| 693135 | | 35.3 | 56.3 |
| 644945 | | 35.3 | 53.4 |
| 715669 | | 35 | 56.8 |
| 697932 | | 35 | 46.8 |
| 681454 | | 35 | 43.2 |
| 324979 | | 35 | 48.9 |

TABLE 5
Compounds Identified Relevant to Prostate Cancer Treatment

| | Mean predicted |
|---|---|
| NSC # | response rate |
| 378475 | 31.1% |
| 668485 | 30.5% |
| 681143 | 30.5% |
| 674603 | 30.5% |
| 638440 | 30.5% |
| 67690 | 30.5% |
| 708375 | 30.5% |
| 701671 | 30.5% |
| 239375 | 30.5% |
| 322921 | 30.5% |
| 668265 | 29.9% |
| 59270 | 29.9% |
| 657749 | 29.9% |
| 714379 | 29.9% |
| 624975 | 29.9% |
| 687801 | 29.9% |
| 664213 | 29.9% |
| 686324 | 29.9% |
| 699452 | 29.9% |
| 721394 | 29.9% |
| 724440 | 29.9% |
| 118994 | 29.4% |
| 685485 | 29.4% |
| 668324 | 29.4% |
| 201434 | 29.4% |
| 349644 | 29.4% |
| 603108 | 29.4% |
| 662452 | 29.4% |
| 674620 | 29.4% |
| 740 | 29.4% |
| 211685 | 29.4% |
| 704288 | 29.4% |
| 382044 | 29.4% |
| 718650 | 29.4% |
| 637651 | 29.4% |
| 637399 | 29.4% |
| 723513 | 29.4% |
| 693633 | 29.4% |
| 726449 | 29.4% |
| 671881 | 29.4% |
| 715067 | 29.4% |
| 715175 | 29.4% |
| 648543 | 29.4% |
| 721622 | 29.4% |
| 64875 | 29.4% |
| 670558 | 29.4% |
| 684989 | 29.4% |
| 35489 | 28.8% |
| 706032 | 28.8% |
| 600392 | 28.8% |
| 349856 | 28.8% |
| 625156 | 28.8% |
| 657561 | 28.8% |
| 631306 | 28.8% |
| 645159 | 28.8% |
| 680399 | 28.8% |
| 698229 | 28.8% |
| 732827 | 28.8% |
| 661416 | 28.8% |
| 630511 | 28.8% |
| 687520 | 28.8% |
| 679749 | 28.8% |
| 683661 | 28.8% |
| 665101 | 28.8% |
| 665604 | 28.8% |
| 704341 | 28.8% |
| 691033 | 28.8% |
| 718722 | 28.8% |
| 637126 | 28.8% |
| 637462 | 28.8% |
| 626482 | 28.8% |
| 686342 | 28.8% |
| 643813 | 28.8% |
| 693714 | 28.8% |
| 669142 | 28.8% |
| 169471 | 28.8% |
| 38186 | 28.8% |
| 261045 | 28.8% |
| 710556 | 28.8% |
| 166637 | 28.8% |
| 715230 | 28.8% |
| 720199 | 28.8% |
| 670875 | 28.8% |
| 658874 | 28.2% |
| 692656 | 28.2% |
| 628910 | 28.2% |
| 706980 | 28.2% |
| 706739 | 28.2% |
| 331935 | 28.2% |
| 716182 | 28.2% |
| 716272 | 28.2% |
| 618757 | 28.2% |
| 685125 | 28.2% |
| 15889 | 28.2% |
| 729608 | 28.2% |
| 668331 | 28.2% |
| 668254 | 28.2% |
| 668264 | 28.2% |
| 201438 | 28.2% |
| 349051 | 28.2% |
| 36806 | 28.2% |
| 709079 | 28.2% |
| 680410 | 28.2% |
| 98949 | 28.2% |
| 678156 | 28.2% |
| 712206 | 28.2% |
| 712182 | 28.2% |
| 682433 | 28.2% |
| 698148 | 28.2% |
| 638410 | 28.2% |
| 159631 | 28.2% |
| 661440 | 28.2% |
| 630609 | 28.2% |
| 656954 | 28.2% |
| 687808 | 28.2% |
| 683426 | 28.2% |
| 665918 | 28.2% |
| 708550 | 28.2% |
| 704874 | 28.2% |
| 704120 | 28.2% |
| 382046 | 28.2% |
| 382049 | 28.2% |
| 691566 | 28.2% |
| 718028 | 28.2% |
| 702984 | 28.2% |
| 351105 | 28.2% |
| 693867 | 28.2% |
| 693442 | 28.2% |
| 10460 | 28.2% |
| 669995 | 28.2% |
| 727679 | 28.2% |
| 671465 | 28.2% |
| 671097 | 28.2% |
| 671118 | 28.2% |
| 671113 | 28.2% |
| 311152 | 28.2% |
| 676179 | 28.2% |
| 710393 | 28.2% |
| 699164 | 28.2% |
| 677256 | 28.2% |
| 677937 | 28.2% |
| 717093 | 28.2% |
| 632841 | 28.2% |
| 614554 | 28.2% |
| 111702 | 28.2% |
| 715971 | 28.2% |
| 715524 | 28.2% |
| 715083 | 28.2% |
| 694879 | 28.2% |
| 694501 | 28.2% |
| 138780 | 28.2% |
| 266046 | 28.2% |
| 670229 | 28.2% |
| 174121 | 27.7% |
| 54044 | 27.7% |
| 703776 | 27.7% |
| 26647 | 27.7% |
| 26382 | 27.7% |
| 204936 | 27.7% |
| 73013 | 27.7% |
| 618261 | 27.7% |
| 685981 | 27.7% |
| 613238 | 27.7% |
| 348948 | 27.7% |
| 642492 | 27.7% |
| 649565 | 27.7% |
| 668366 | 27.7% |
| 309401 | 27.7% |
| 131238 | 27.7% |
| 625154 | 27.7% |
| 663855 | 27.7% |
| 709137 | 27.7% |
| 709969 | 27.7% |
| 709925 | 27.7% |
| 4623 | 27.7% |
| 631521 | 27.7% |
| 631527 | 27.7% |
| 88054 | 27.7% |
| 662788 | 27.7% |
| 674131 | 27.7% |
| 674178 | 27.7% |
| 674913 | 27.7% |
| 680935 | 27.7% |
| 680717 | 27.7% |
| 678036 | 27.7% |
| 129957 | 27.7% |
| 707181 | 27.7% |
| 707079 | 27.7% |
| 682815 | 27.7% |
| 682689 | 27.7% |
| 310365 | 27.7% |
| 661938 | 27.7% |
| 661939 | 27.7% |
| 150446 | 27.7% |
| 656210 | 27.7% |
| 355063 | 27.7% |
| 363952 | 27.7% |
| 687803 | 27.7% |

TABLE 6
Compounds Identified Relevant to Melanoma Treatment

| NSC # | Mean predicted response rate |
|---|---|
| 241240 | 50.0% |
| 654236 | 48.6% |
| 333843 | 48.6% |
| 719738 | 48.6% |
| 665741 | 48.6% |
| 609699 | 47.1% |
| 688363 | 47.1% |
| 643027 | 47.1% |
| 708563 | 47.1% |
| 681640 | 47.1% |
| 670294 | 45.7% |
| 653620 | 45.7% |
| 235178 | 45.7% |
| 718553 | 45.7% |
| 603976 | 45.7% |
| 26074 | 45.7% |
| 634770 | 45.7% |
| 670963 | 44.3% |
| 720557 | 44.3% |
| 629286 | 44.3% |
| 671311 | 44.3% |
| 708546 | 44.3% |
| 708446 | 44.3% |
| 749 | 44.3% |
| 707040 | 44.3% |
| 374980 | 44.3% |
| 680537 | 44.3% |
| 612115 | 44.3% |
| 681226 | 44.3% |
| 658777 | 44.3% |
| 684074 | 42.9% |
| 715471 | 42.9% |
| 156216 | 42.9% |
| 722568 | 42.9% |
| 717853 | 42.9% |
| 666605 | 42.9% |
| 671165 | 42.9% |
| 38525 | 42.9% |
| 675256 | 42.9% |
| 664908 | 42.9% |
| 664173 | 42.9% |
| 672131 | 42.9% |
| 683636 | 42.9% |
| 157389 | 42.9% |
| 355256 | 42.9% |
| 681069 | 42.9% |
| 705899 | 42.9% |
| 705584 | 42.9% |
| 685529 | 42.9% |
| 269754 | 42.9% |
| 703443 | 42.9% |
| 703033 | 42.9% |
| 658296 | 42.9% |
| 720767 | 41.4% |
| 711873 | 41.4% |
| 717862 | 41.4% |
| 677959 | 41.4% |
| 699742 | 41.4% |
| 666377 | 41.4% |
| 639857 | 41.4% |
| 688500 | 41.4% |
| 669814 | 41.4% |
| 723742 | 41.4% |
| 723171 | 41.4% |
| 119875 | 41.4% |
| 718153 | 41.4% |
| 689081 | 41.4% |
| 655903 | 41.4% |
| 708564 | 41.4% |
| 667721 | 41.4% |
| 667934 | 41.4% |
| 641245 | 41.4% |
| 665349 | 41.4% |
| 714391 | 41.4% |
| 67586 | 41.4% |
| 712914 | 41.4% |
| 680338 | 41.4% |
| 674997 | 41.4% |
| 645646 | 41.4% |
| 372155 | 41.4% |
| 405995 | 41.4% |
| 642198 | 41.4% |
| 642409 | 41.4% |
| 703119 | 41.4% |
| 616356 | 40.0% |
| 695632 | 40.0% |
| 609397 | 40.0% |
| 670225 | 40.0% |
| 715565 | 40.0% |
| 673797 | 40.0% |
| 619679 | 40.0% |
| 677923 | 40.0% |
| 677200 | 40.0% |
| 666737 | 40.0% |
| 639829 | 40.0% |
| 659181 | 40.0% |
| 727730 | 40.0% |
| 669999 | 40.0% |
| 23925 | 40.0% |
| 173931 | 40.0% |
| 718516 | 40.0% |
| 655898 | 40.0% |
| 664979 | 40.0% |
| 667933 | 40.0% |
| 667948 | 40.0% |
| 641233 | 40.0% |
| 409962 | 40.0% |
| 683437 | 40.0% |
| 679678 | 40.0% |
| 687790 | 40.0% |
| 660633 | 40.0% |
| 660632 | 40.0% |
| 136476 | 40.0% |
| 656178 | 40.0% |
| 624254 | 40.0% |
| 714381 | 40.0% |
| 159065 | 40.0% |
| 712821 | 40.0% |
| 713197 | 40.0% |
| 98828 | 40.0% |
| 680223 | 40.0% |
| 290494 | 40.0% |
| 657782 | 40.0% |
| 612116 | 40.0% |
| 604976 | 40.0% |
| 681730 | 40.0% |
| 116555 | 40.0% |
| 716887 | 40.0% |
| 716296 | 40.0% |
| 716697 | 40.0% |
| 692392 | 40.0% |
| 617668 | 40.0% |
| 174589 | 40.0% |
| 670806 | 38.6% |
| 670315 | 38.6% |
| 720486 | 38.6% |
| 720765 | 38.6% |
| 715224 | 38.6% |
| 715592 | 38.6% |
| 673190 | 38.6% |
| 673788 | 38.6% |
| 166637 | 38.6% |
| 710895 | 38.6% |
| 676181 | 38.6% |
| 676591 | 38.6% |
| 644211 | 38.6% |
| 671043 | 38.6% |
| 383468 | 38.6% |
| 650771 | 38.6% |
| 123147 | 38.6% |
| 123127 | 38.6% |
| 274539 | 38.6% |
| 693443 | 38.6% |
| 2979 | 38.6% |
| 83265 | 38.6% |
| 112200 | 38.6% |
| 723518 | 38.6% |
| 119875 | 38.6% |
| 686560 | 38.6% |
| 621456 | 38.6% |
| 82151 | 38.6% |
| 689719 | 38.6% |
| 672230 | 38.6% |
| 672059 | 38.6% |
| 672058 | 38.6% |
| 672556 | 38.6% |
| 708425 | 38.6% |
| 667384 | 38.6% |
| 667924 | 38.6% |
| 665072 | 38.6% |
| 679744 | 38.6% |
| 679743 | 38.6% |
| 687106 | 38.6% |
| 157390 | 38.6% |
| 680073 | 38.6% |
| 674080 | 38.6% |
| 646860 | 38.6% |
| 90810 | 38.6% |
| 681528 | 38.6% |
| 31660 | 38.6% |
| 375726 | 38.6% |
| 106408 | 38.6% |
| 106648 | 38.6% |
| 716091 | 38.6% |
| 269753 | 38.6% |
| 692656 | 38.6% |
| 725051 | 37.1% |
| 725100 | 37.1% |
| 695788 | 37.1% |
| 654705 | 37.1% |
| 684565 | 37.1% |
| 327993 | 37.1% |
| 670323 | 37.1% |
| 670013 | 37.1% |
| 720495 | 37.1% |
| 3060 | 37.1% |
| 648583 | 37.1% |
| 694212 | 37.1% |

TABLE 7
Compounds Identified Relevant to Pancreatic Cancer Treatment

| NSC # | Mean predicted response rate |
|---|---|
| 710019 | 40.8% |
| 658857 | 40.8% |
| 733892 | 38.8% |
| 715682 | 38.8% |
| 710779 | 38.8% |
| 708416 | 38.8% |
| 698966 | 38.8% |
| 693561 | 38.8% |
| 683920 | 38.8% |
| 679495 | 38.8% |
| 668327 | 38.8% |
| 667739 | 38.8% |
| 641296 | 38.8% |
| 633530 | 38.8% |
| 606398 | 38.8% |
| 44185 | 38.8% |
| 36002 | 38.8% |
| 731130 | 36.7% |
| 726246 | 36.7% |
| 725118 | 36.7% |
| 724291 | 36.7% |
| 722974 | 36.7% |
| 717036 | 36.7% |
| 715775 | 36.7% |
| 714379 | 36.7% |
| 710352 | 36.7% |
| 709587 | 36.7% |
| 708810 | 36.7% |
| 708075 | 36.7% |
| 703548 | 36.7% |
| 701663 | 36.7% |
| 698959 | 36.7% |
| 697862 | 36.7% |
| 697218 | 36.7% |
| 695935 | 36.7% |
| 694879 | 36.7% |
| 694482 | 36.7% |
| 693565 | 36.7% |
| 689137 | 36.7% |
| 688104 | 36.7% |
| 687308 | 36.7% |
| 686403 | 36.7% |
| 685981 | 36.7% |
| 685793 | 36.7% |
| 685504 | 36.7% |
| 685227 | 36.7% |
| 683791 | 36.7% |
| 683376 | 36.7% |
| 676385 | 36.7% |
| 674603 | 36.7% |
| 673651 | 36.7% |
| 670802 | 36.7% |
| 670227 | 36.7% |
| 668331 | 36.7% |
| 667252 | 36.7% |
| 665894 | 36.7% |
| 662124 | 36.7% |
| 659332 | 36.7% |
| 659166 | 36.7% |
| 657829 | 36.7% |
| 641241 | 36.7% |
| 640071 | 36.7% |
| 633403 | 36.7% |
| 632841 | 36.7% |
| 630602 | 36.7% |
| 630004 | 36.7% |
| 626879 | 36.7% |
| 625543 | 36.7% |
| 622114 | 36.7% |
| 611271 | 36.7% |
| 382044 | 36.7% |
| 375086 | 36.7% |
| 325306 | 36.7% |
| 278571 | 36.7% |
| 248040 | 36.7% |
| 165572 | 36.7% |
| 101212 | 36.7% |
| 92937 | 36.7% |
| 90829 | 36.7% |
| 88054 | 36.7% |
| 87221 | 36.7% |
| 76712 | 36.7% |
| 38876 | 36.7% |
| 19024 | 36.7% |
| 729797 | 34.7% |
| 724350 | 34.7% |
| 724063 | 34.7% |
| 724005 | 34.7% |
| 720147 | 34.7% |
| 718519 | 34.7% |
| 717187 | 34.7% |
| 715778 | 34.7% |
| 715559 | 34.7% |
| 715176 | 34.7% |
| 713599 | 34.7% |
| 710718 | 34.7% |
| 710608 | 34.7% |
| 709971 | 34.7% |
| 709858 | 34.7% |
| 709002 | 34.7% |
| 707182 | 34.7% |
| 707040 | 34.7% |
| 706989 | 34.7% |
| 704561 | 34.7% |
| 703122 | 34.7% |
| 702115 | 34.7% |
| 701099 | 34.7% |
| 700274 | 34.7% |
| 699832 | 34.7% |
| 699726 | 34.7% |
| 699428 | 34.7% |
| 699251 | 34.7% |
| 699023 | 34.7% |
| 698678 | 34.7% |
| 698164 | 34.7% |
| 697892 | 34.7% |
| 697530 | 34.7% |
| 695938 | 34.7% |
| 695043 | 34.7% |
| 694266 | 34.7% |
| 694218 | 34.7% |
| 693714 | 34.7% |
| 693637 | 34.7% |
| 691696 | 34.7% |
| 691277 | 34.7% |
| 691250 | 34.7% |
| 689278 | 34.7% |
| 687368 | 34.7% |
| 687002 | 34.7% |
| 685826 | 34.7% |
| 685418 | 34.7% |
| 684439 | 34.7% |
| 683887 | 34.7% |
| 683830 | 34.7% |
| 683140 | 34.7% |
| 683044 | 34.7% |
| 682504 | 34.7% |
| 682138 | 34.7% |
| 681127 | 34.7% |
| 680770 | 34.7% |
| 680399 | 34.7% |
| 679742 | 34.7% |
| 679003 | 34.7% |
| 679002 | 34.7% |
| 678918 | 34.7% |
| 678501 | 34.7% |
| 677398 | 34.7% |
| 677296 | 34.7% |
| 677240 | 34.7% |
| 676496 | 34.7% |
| 675967 | 34.7% |
| 675593 | 34.7% |
| 674456 | 34.7% |
| 674215 | 34.7% |
| 673790 | 34.7% |
| 673611 | 34.7% |
| 672426 | 34.7% |
| 671814 | 34.7% |
| 671809 | 34.7% |
| 671119 | 34.7% |
| 671031 | 34.7% |
| 670314 | 34.7% |
| 669739 | 34.7% |
| 668394 | 34.7% |
| 668330 | 34.7% |
| 667707 | 34.7% |
| 667057 | 34.7% |
| 666765 | 34.7% |
| 665971 | 34.7% |
| 665804 | 34.7% |
| 665603 | 34.7% |
| 665333 | 34.7% |
| 665288 | 34.7% |
| 665079 | 34.7% |
| 664908 | 34.7% |
| 664283 | 34.7% |
| 662199 | 34.7% |
| 661238 | 34.7% |
| 659468 | 34.7% |
| 659348 | 34.7% |
| 658484 | 34.7% |
| 658144 | 34.7% |
| 658114 | 34.7% |
| 658009 | 34.7% |
| 657758 | 34.7% |
| 657174 | 34.7% |
| 650770 | 34.7% |
| 646603 | 34.7% |
| 641691 | 34.7% |
| 641297 | 34.7% |
| 639541 | 34.7% |
| 637128 | 34.7% |
| 633253 | 34.7% |
| 632877 | 34.7% |
| 627050 | 34.7% |
| 626482 | 34.7% |
| 626307 | 34.7% |
| 624659 | 34.7% |

RESULTS (COMBINATION OF AGENTS): Evaluation of In Vitro Drug Sensitivity of Human Bladder Cell Lines to Single Agents

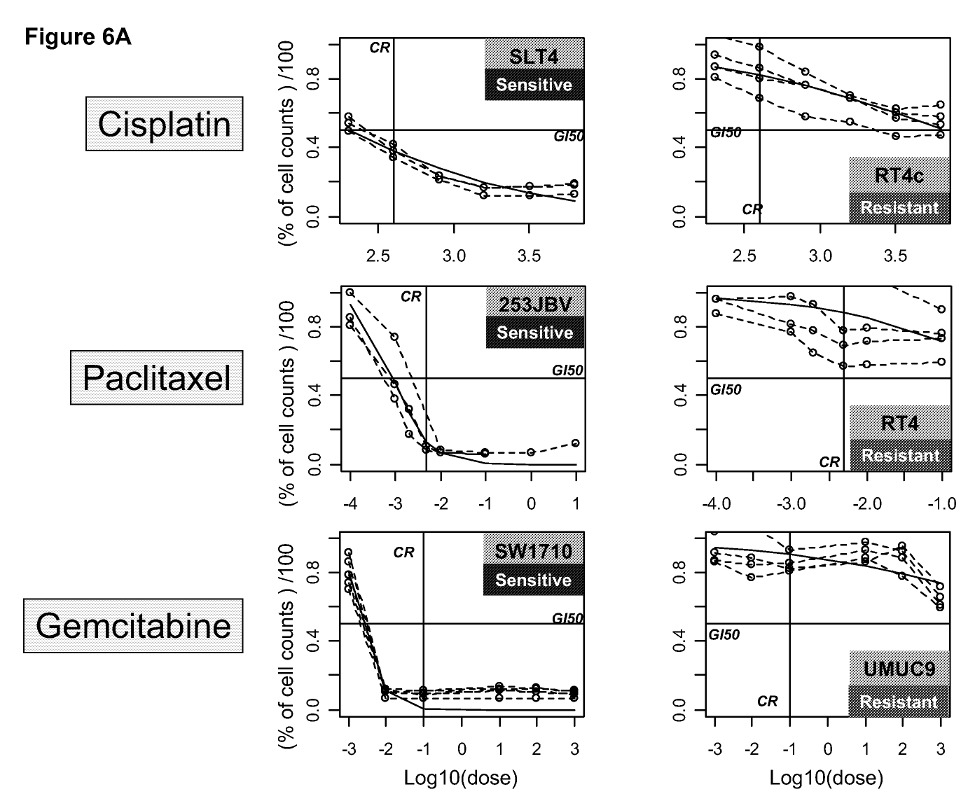

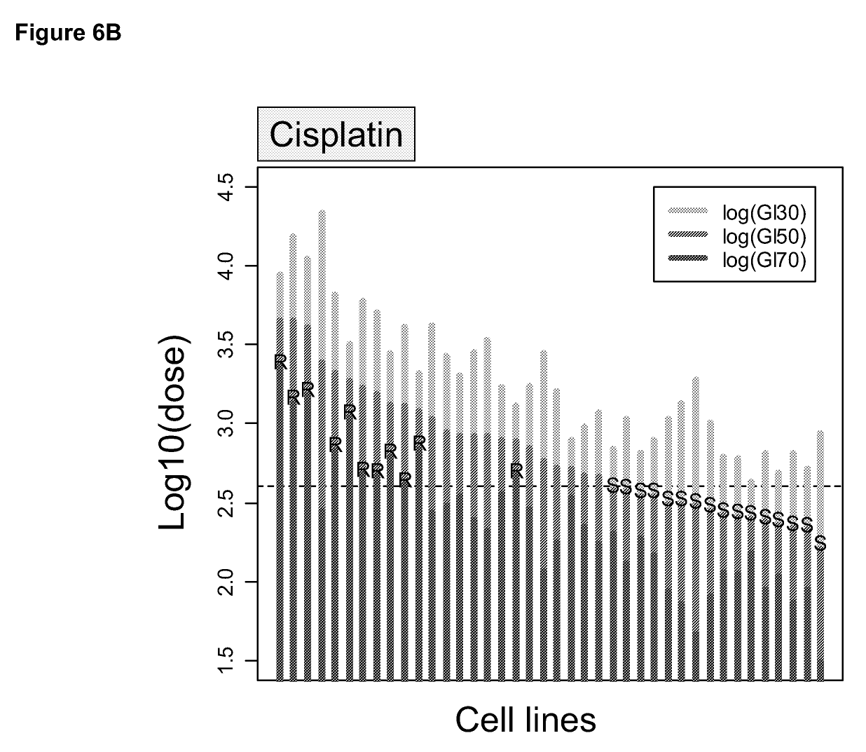

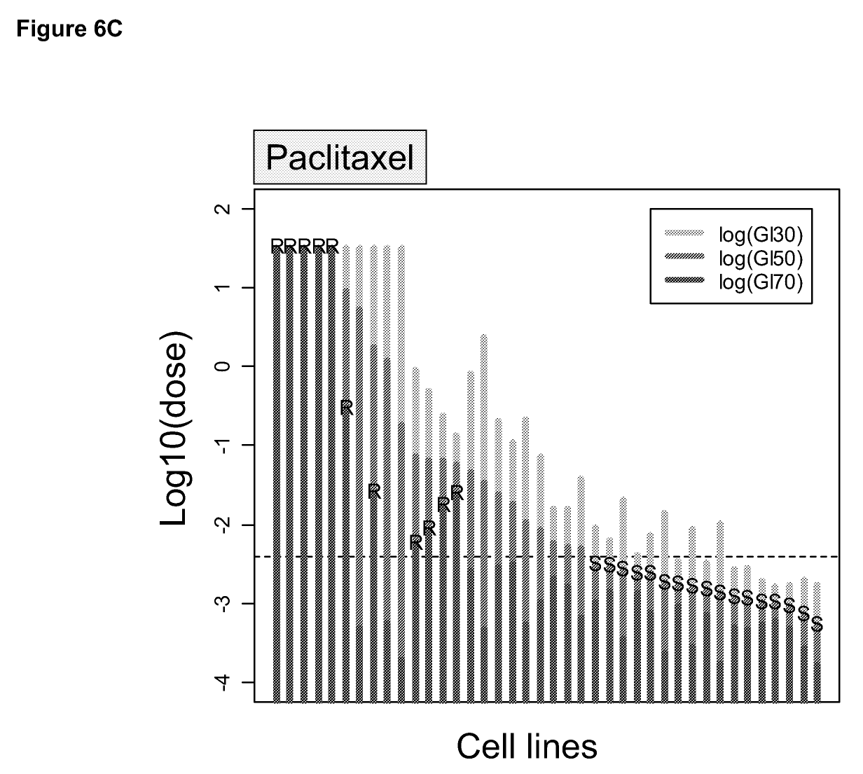

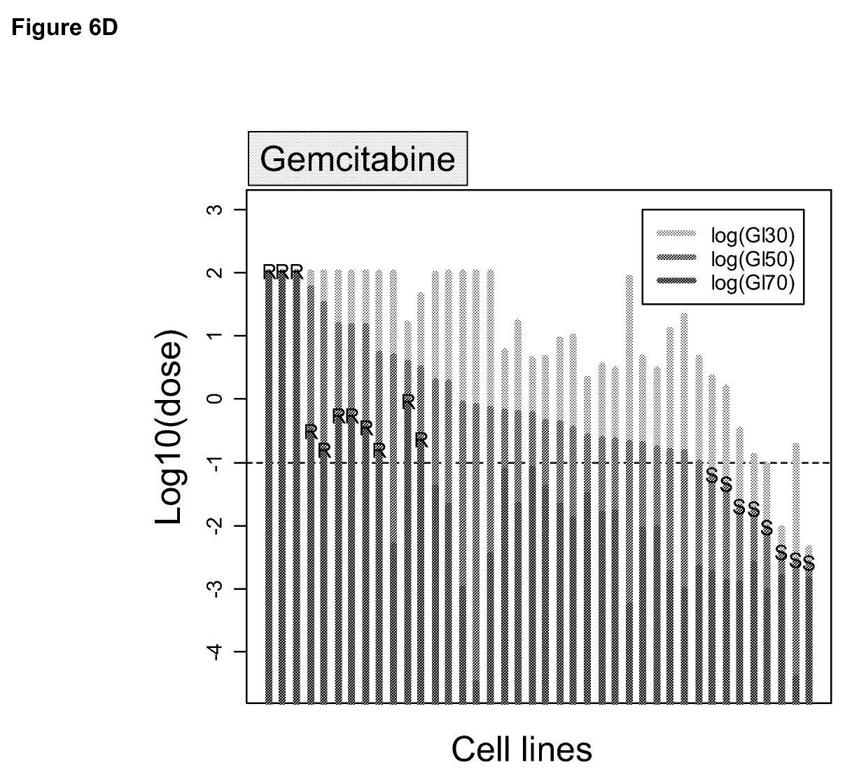

To approach the development of molecular models of chemotherapeutic sensitivity in human bladder cancer, we focused on a well-defined series of 40 urothelial cell lines for which we could measure sensitivity to relevant chemotherapeutic agents in vitro and correlate these responses with global measurements of gene expression. The in vitro sensitivity of these 40 bladder cancer cell lines to cisplatin, paclitaxel, and gemcitabine was carried out as described in Materials and Methods. Typical dose response curves for representative sensitive and resistant cell lines are shown for each agent in FIG. 6A. The cell lines were then divided into three groups, sensitive, intermediate, and resistant, based on GI estimates and the criterion dose (CR; defined in Materials and Methods). FIGS. 6B-6D show the log 10(GI30), log 10(GI50), and log 10(GI70) of the 40 cell lines for each of the agents. For cisplatin, we identified 16 sensitive and 11 resistant cell lines (FIG. 6B); 17 sensitive and 11 resistant cell lines for Paclitaxel (FIG. 6C), and 8 sensitive and 11 resistant for Gemcitabine (FIG. 6D). Cell lines that did not meet the “sensitive/resistant” criteria were excluded from further analyses. For some cell lines, log(GI) values could not be estimated due to flat response curves in nonlinear regression model fitting; thus, these cell lines' log(GI) values were thresholded at the maximum dose concentration and were classified as resistant.

Prediction Models for Individual Drug Sensitivity

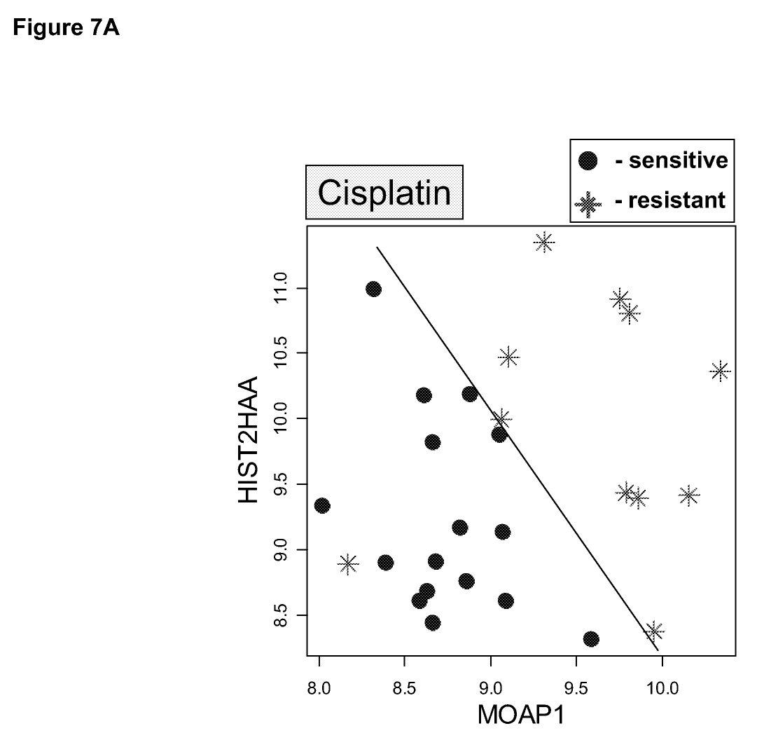

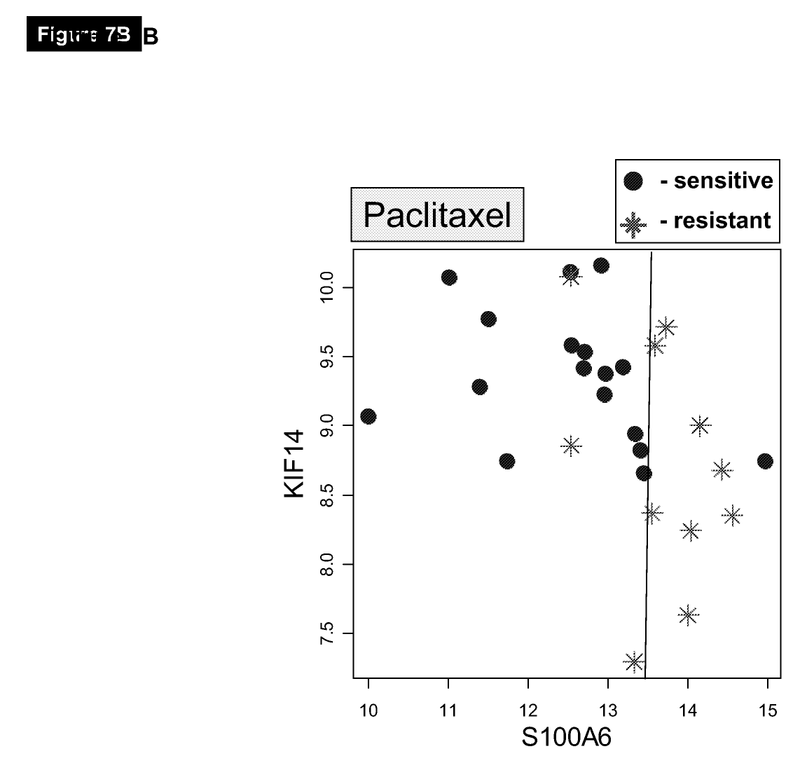

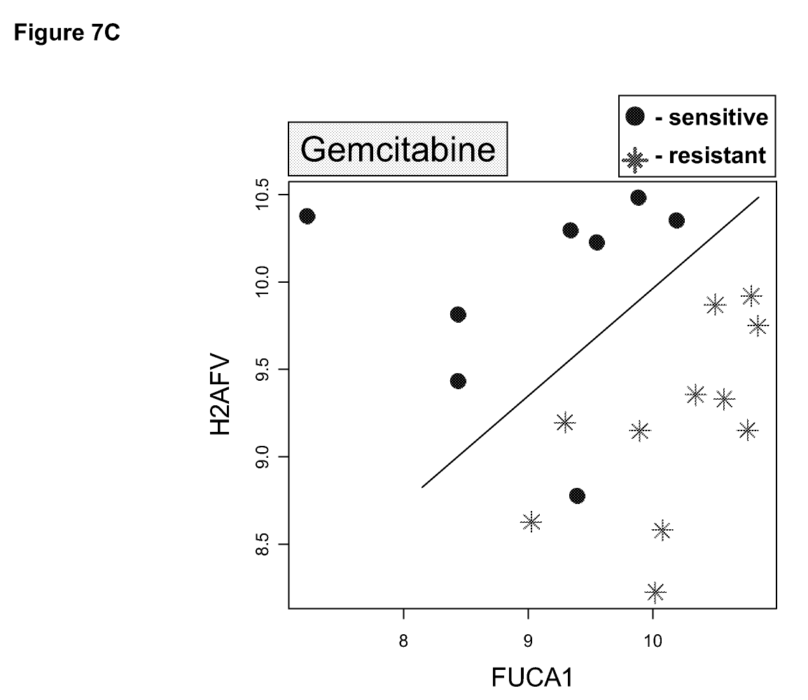

We used the MiPP approach to identify models comprised of gene transcript levels that predicted sensitivity to cisplatin, paclitaxel and gemcitabine (see Materials and Methods). For cisplatin and paclitaxel, we identified three prediction models that met the criteria for selection of sensitive and resistant cells (i.e. with the lower 5% sMiPP>0.5); for Gemcitabine, we identified only one model that met these criteria (Table 8A); The selection and order of these models were based on the 5% sMiPP, so was the order of the models. The mean sMiPPs among the three models for Cisplatin were 0.820-0.858, with mean misclassification rates of 5.4-6.9% (prediction accuracies=93.1 to 94.6%), based on independent-set cross-validation as described (13). The prediction performance of Paclitaxel models was similar to that of Cisplatin with mean misclassification rates of between 4.1-7.1% and mean sMiPPs of 0.830 to 0.910. For Gemcitabine, we identified a single model with an associated error rate of 9.6% and sMiPP of 0.742. In addition to the performance calculations above, the utility of these gene models in predicting the responsiveness of these drugs can be appreciated by plotting the expression intensities (log 2 scale) of the first two genes in each of our gene prediction models, adding each classification decision line to show the relationship with our classification modeling (FIGS. 7A-C).

Prediction Models for Combination Drug Sensitivity

Given the ability to predict single drug efficacy in vitro, we next asked whether this approach could be used to predict the efficacy of the three commonly used drug doublet combinations in the same types of cells. We applied the same basic MiPP approach, but averaged the posterior probabilities from each of the models in cases where more than one model met the CR (i.e. for paclitaxel and cisplatin) and then computed the chemosensitivity probability for a given drug. If the combined posterior probability of chemosensitivity for a drug combination was >0.75, a cell line was predicted to be sensitive to that drug combination.

We evaluated the performance of these in silico predictions by randomly selecting fifteen of the 40 bladder carcinoma cell lines, attempting to roughly balance the numbers of predicted sensitive and resistant cell lines across the three drug combinations. We used the single drug criteria dose (CR) and exposed cells to both drugs simultaneously. The growth of cell lines exposed to the drug combinations compared to control (no drug) was expected to be <55% for sensitive and >55% for resistant cell lines at these doses.

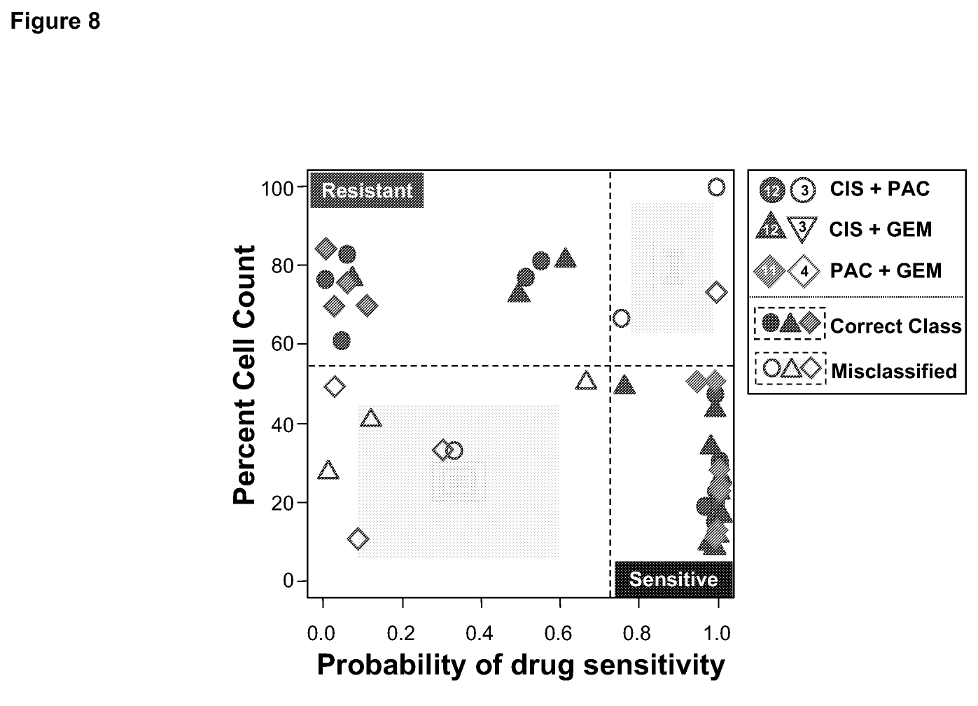

Overall, 35 of the 45 predictions were correct (binomial test p-value=0.0002, Table 8B and FIG. 8). Twelve of fifteen cell lines (80%, binomial test p-value=0.03) were predicted correctly for the Cisplatin-Paclitaxel combination. Of the three misclassified cell lines, one sensitive line was predicted as resistant, and two resistant cell lines were predicted as sensitive. For the Cisplatin-Gemcitabine combination, 12/15 lines were also predicted correctly; three sensitive cell lines were incorrectly predicted as resistant (80% accuracy, binomial test p-value=0.03). Finally, for the combination of Paclitaxel and Gemcitabine, 11/15 lines were correctly classified; three sensitive and one resistant cell lines were misclassified as resistant and sensitive, respectively (73% accuracy, binomial test p-value=0.11).

Potential Synergistic Activities with Combination Treatments

In clinical practice, combination treatments significantly outperform single-drug counterparts in treating different types of cancer, either by additive or synergistic drug action. To this end, we found that 7 of 19 (37%) cell lines that were predicted as resistant to the drug combination used were indeed sensitive to the combination when tested, even though the cells were not sensitive to the single compounds of the combination. For example, in the combination treatment of cisplatin and gemcitabine, all three misclassified cases turned out to be predicted resistant cell lines being in fact sensitive when tested. In contrast, fewer (12%: 3/26) predicted sensitive cell lines to the drug combination were found to be resistant to the combination (two-sample proportion test p=0.049).

TABLE 8A
Best gene prediction models for single drug chemosensitivity
response prediction to cisplatin, paclitaxel, and gemcitabine. Up to three models were
selected with the selection criterion 5% sMiPP > 0.5.

| | | | |
|---|---|---|---|
| Models | Probe set ID | Gene symbol | Gene title |

Cisplatin

| | | | |
|---|---|---|---|
| Model 1 | 212508_at | MOAP1 | modulator of apoptosis 1 |
| mean ER = 0.069 | 218280_x_at | HIST2H2AA | histone 2, H2aa |
| mean sMiPP = 0.858 | 222275_at | MRPS30 | mitochondrial ribosomal protein S30 |
| lower | | | |
| 5% sMiPP = 0.771 | 211573_x_at | TGM2 | transglutaminase 2 |
| Model 2 | 212508_at | MOAP1 | modulator of apoptosis 1 |
| mean ER = 0.054 | 203323_at | CAV 2 | caveolin 2 |
| mean sMiPP = 0.860 | 208885_at | LCP1 | Lymphocyte cytosolic protein 1 (L- |
| lower | | | plastin) |
| 5% sMiPP = 0.730 | | | |
| Model 3 | 211559_s_at | CCNG2 | cyclin G2 |
| mean ER = 0.066 | 212094_at | PEG10 | paternally expressed 10 |
| mean sMiPP = 0.820 | 221029_s_at | WNT5B | wingless-type MMTV integration |
| lower | | | site family, member 5B /// wingless- |
| 5% sMiPP = 0.715 | | | type MMTV integration site family, |
| | | | member 5B |

Paclitaxel

| | | | |
|---|---|---|---|
| Model 1 | 214858_at | GPC1 | Glypican 1 |
| mean ER = 0.041 | 201860_s_at | PLAT | plasminogen activator, tissue |
| mean sMiPP = 0.910 | 201317_s_at | PSMA2 | proteasome (prosome, macropain) |
| lower | | | subunit, alpha type, 2 |
| 5% sMiPP = 0.788 | 211812_s_at | B3GALT3 | UDP-Gal: betaGlcNAc beta 1,3- |
| | | | galactosyltransferase, polypeptide 3 |
| | 204557_s_at | DZIP1 | DAZ interacting protein 1 |
| Model 2 | 217728_at | S100A6 | S100 calcium binding protein A6 |
| mean ER = 0.051 | | | (calcyclin) |
| mean sMiPP = 0.877 | | | |
| lower | | | |
| 5% sMiPP = 0.770 | | | |
| | 206364_at | KIF14 | kinesin family member 14 |
| | 203741_s_at | ADCY7 | adenylate cyclase 7 |
| | 203438_at | STC2 | stanniocalcin 2 |
| | 201105_at | LGALS1 | lectin, galactoside-binding, soluble, |
| | | | 1 (galectin 1) |
| Model 3 | 206059_at | ZNF91 | zinc finger protein 91 (HPF7, |
| mean ER = 0.071 | | | HTF10) |
| mean sMiPP = 0.830 | | | |
| lower | | | |
| 5% sMiPP = 0.746 | | | |
| | 209310_s_at | CASP4 | caspase 4, apoptosis-related cysteine |
| | | | protease |
| | 213849_s_at | PPP2R2B | protein phosphatase 2 (formerly |
| | | | 2A), regulatory subunit B (PR 52), |
| | | | beta isoform |
| | 202591_s_at | SBP1 | single-stranded DNA binding |
| | | | protein 1 |

Gemcitabine

| | | | |
|---|---|---|---|
| Model 1 | 202838_at | FUCA1 | fucosidase, alpha-L-1, tissue |
| mean ER = 0.096 | | | |
| mean sMiPP = 0.742 | | | |
| lower | | | |
| 5% sMiPP = 0.582 | | | |
| | 212206_s_at | H2AFV | H2A histone family, member V |

Table 8B. Predicted sensitivity probabilities to combination therapy and validation in fifteen urothelial cancer cell lines. The growth inhibition of the combination drug treatment experiments (% of cell count in cells not exposed to drug) was obtained using the dose concentrations: Cisplatin log 10(400 ng/ml), Paclitaxel log₁₀(0.005 μM), and Gemcitabine log₁₀(0.1 μM). A cell line with the larger posterior probability (PP) is more likely to be a sensitive. Single-drug posterior probabilities were obtained by averaging posterior probabilities if there were more than one model, and the combined posterior probability is 1-Pr (Resistant by Cisplatin)×Pr (Resistant by Paclitaxel). (Predicted as Sensitive if PP>0.75 and as Resistant if PP<0.75). Predicted sensitive (denoted by S) or resistant (denoted by R) cell lines to the combination pairs of three drug treatments. * Indicates misclassified samples when compared to in vitro evaluation of drug combinations.

| | | | | % OF CELL COUNT | | | PROBABILITY | | | PREDICTION | | |
|---|---|---|---|---|---|---|---|---|---|---|---|---|
| CELL | PROBABILITY | | | CIS + | CIS + | PAC + | CIS + | CIS + | PAC + | CIS + | CIS + | PAC + |
| LINES | CIS | PAC | GEM | PAC | GEM | GEM | PAC | GEM | GEM | PAC | GEM | GEM |
| 253JBV | 0.90 | 1.00 | 0.93 | 15 | 25 | 22 | 1.00 | 0.99 | 1.00 | S | S | S |
| 253JLaval | 0.54 | 1.00 | 0.16 | 100 | 81 | 73 | 1.00 | 0.61 | 1.00 | S* | R | S* |
| 253JP | 0.98 | 1.00 | 0.93 | 20 | 16 | 27 | 1.00 | 1.00 | 1.00 | S | S | S |
| CRL7833 | 0.97 | 0.02 | 0.07 | 19 | 10 | 11 | 0.97 | 0.98 | 0.09 | S | S | R* |
| HT1197 | 0.50 | 0.03 | 0.00 | 77 | 72 | 49 | 0.51 | 0.50 | 0.03 | R | R | R* |
| HT1376 | 0.54 | 0.03 | 0.28 | 81 | 50 | 33 | 0.55 | 0.67 | 0.30 | R | R* | R* |
| HU456 | 0.95 | 0.86 | 0.63 | 48 | 34 | 51 | 0.99 | 0.98 | 0.95 | S | S | S |
| J82 | 0.33 | 0.00 | 0.99 | 32 | 43 | 51 | 0.33 | 0.99 | 0.99 | R* | S | S |
| JON | 0.02 | 0.01 | 0.10 | 61 | 41 | 70 | 0.03 | 0.12 | 0.11 | R | R* | R |
| MGHU3 | 0.01 | 0.01 | 0.01 | 76 | 28 | 84 | 0.01 | 0.01 | 0.01 | R | R* | R |
| RT4 | 0.05 | 0.00 | 0.02 | 83 | 77 | 70 | 0.05 | 0.08 | 0.02 | R | R | R |
| T24T | 1.00 | 0.99 | 0.30 | 29 | 11 | 12 | 1.00 | 1.00 | 0.99 | S | S | S |
| TCCSUP | 0.75 | 0.02 | 0.05 | 64 | 49 | 76 | 0.76 | 0.76 | 0.06 | S* | S | R |
| UMUC3 | 0.99 | 0.99 | 1.00 | 30 | 21 | 21 | 1.00 | 1.00 | 1.00 | S | S | S |
| UMUC6 | 0.90 | 0.99 | 0.91 | 22 | 8 | 13 | 1.00 | 0.99 | 1.00 | S | S | S |

TABLE 9
Validation of combination COXEN prediction. We validated
combination COXEN prediction against an independent panel of 43 lymphoma patients
treated with CHOP-like regimen for individual agents (cyclophosphamide, doxorubicin,
vincristine, and prednisone). The results of this validation are shown below in Table 9.
All single agents' prediction except for prednisone were statistically significant:
p-value = 0.006 for cyclophosphamide, 0.029 for doxorubicin, and 0.005 for
vincristine. The NCI-60 screening data of prednisone was not informative, not
showing meaningful agent activity differences (in GI50 values) on the NCI-60.
Consequently, the overall combination drug activities between responders and
non-responders were predicted without prednisone prediction, yet still statistically
extremely significant (two-sample t-test p-value = 0.0001).

| | DRUG | | | |
|---|---|---|---|---|
| | CYC(−4.0M) | DOX(−4.6M) | VIN(−3.0M) | Prob(Responder) |
| | #(identified genes) | | | |
| | 170 | 100 | 10 | |
| res | 0.52995885 | 0.46925353 | 0.58613482 | 0.896751944 |
| res | 0.59699201 | 0.475792625 | 0.5641742 | 0.907927545 |
| res | 0.51746887 | 0.574132341 | 0.59566978 | 0.916912403 |
| res | 0.50522372 | 0.579650365 | 0.67999878 | 0.933446457 |
| res | 0.40199212 | 0.565932331 | 0.62106537 | 0.901637706 |
| res | 0.47625359 | 0.53907145 | 0.68920719 | 0.92497161 |
| res | 0.49577668 | 0.544815267 | 0.52437687 | 0.890837473 |
| res | 0.49170725 | 0.533339785 | 0.586864 | 0.902004139 |
| res | 0.47334042 | 0.487905457 | 0.62314277 | 0.898361794 |
| res | 0.45937989 | 0.520329017 | 0.59161586 | 0.894097917 |
| res | 0.51749388 | 0.530175369 | 0.55900396 | 0.900029169 |
| res | 0.58938115 | 0.526878264 | 0.65494656 | 0.932965535 |
| res | 0.57365335 | 0.562384393 | 0.71971989 | 0.947706474 |
| res | 0.5888027 | 0.531835056 | 0.67397077 | 0.937236712 |
| res | 0.54872883 | 0.487732736 | 0.72775102 | 0.937063811 |
| res | 0.57079076 | 0.442411833 | 0.58975106 | 0.901818406 |
| res | 0.59725492 | 0.53012458 | 0.71526927 | 0.946117553 |
| res | 0.49704479 | 0.572847898 | 0.50916428 | 0.894549651 |
| res | 0.51356003 | 0.475781071 | 0.68326202 | 0.919231486 |
| res | 0.4879396 | 0.597663025 | 0.5680166 | 0.911002419 |
| res | 0.49451002 | 0.508401663 | 0.6025105 | 0.901224643 |
| res | 0.51127302 | 0.520646745 | 0.61881678 | 0.910699113 |
| res | 0.51429751 | 0.412163833 | 0.51849253 | 0.862523123 |
| nonres | 0.44033274 | 0.584454564 | 0.51132674 | 0.886350638 |
| nonres | 0.50807519 | 0.486511321 | 0.57678304 | 0.893096317 |
| nonres | 0.46464706 | 0.525608592 | 0.55990805 | 0.88823124 |
| nonres | 0.50642389 | 0.534843447 | 0.49850645 | 0.884862015 |
| nonres | 0.45376786 | 0.496530087 | 0.60458045 | 0.891255097 |
| nonres | 0.4510757 | 0.41595867 | 0.56445027 | 0.860365162 |
| nonres | 0.50065546 | 0.455485125 | 0.53290611 | 0.872996922 |
| nonres | 0.39164146 | 0.474331009 | 0.39639086 | 0.806968682 |
| nonres | 0.47894723 | 0.409247215 | 0.54044158 | 0.858541773 |
| nonres | 0.46422896 | 0.55151089 | 0.47203483 | 0.873136582 |
| nonres | 0.40916142 | 0.572614429 | 0.48102171 | 0.86894974 |
| nonres | 0.41690526 | 0.4423161 | 0.61742679 | 0.875593869 |
| nonres | 0.45796277 | 0.471016963 | 0.73866258 | 0.925067112 |
| nonres | 0.50618399 | 0.403055327 | 0.60209621 | 0.88270559 |
| nonres | 0.54695638 | 0.450512218 | 0.40177368 | 0.851076381 |
| nonres | 0.50185861 | 0.492390896 | 0.57890982 | 0.893522673 |
| nonres | 0.51053904 | 0.431598079 | 0.54109584 | 0.87232802 |
| nonres | 0.45294078 | 0.428993975 | 0.46304023 | 0.832267671 |
| nonres | 0.51097601 | 0.436793847 | 0.64165262 | 0.901303491 |
| nonres | 0.58403758 | 0.604706586 | 0.6520198 | 0.942782588 |
| num(res) >= 0.5 | 14 | 16 | 23 | |
| num(nonres) < 0.5 | 11 | 14 | 6 | |
| mean(res) | 0.519688 | 0.521272549 | 0.61751847 | 0.911700743 |
| mean(nonres) | 0.47786587 | 0.483423967 | 0.54875138 | 0.878070078 |
| t-test | 0.00687605 | 0.029454159 | 0.00543869 | 1.54E−04 |

DISCUSSION: Below we will discuss results using COXEN for single and combination agents. These sections are kept separate for clarity here.

DISCUSSION (SINGLE AGENT): The present invention provides a new algorithm, COXEN, for in silico prediction of chemosensitivity. Disclosed herein are illustrative studies in which COXEN was used (i) to extrapolate from chemosensitivity data on the NCI-60 cancer cell panel to an analogous cell line panel of bladder cancers, (ii) to extrapolate from the NCI-60 to clinical data on a panel of breast cancers, and (iii) to predict sensitivity of the bladder cancers to 45,545 candidate agents on the basis of NCI-60 data. Importantly, in each case the algorithm was run independently of the validating experimental results and not further tuned thereafter. We expect that it will be possible in the future to improve the algorithm and its predictions by learning from the experience gained in applications such as those described here.

In the drug discovery test case, the lead hit identified, NSC637993, was an imidazoacridinone, with structural similarities to such drug classes as the anthracyclines (e.g., doxorubicin), the anthracenediones (e.g., mitoxantrone), and the anthrapyrazoles (e.g., oxantrazole and biantrazole), which are known to intercalate in DNA and inhibit DNA topoisomerase II. An almost identical compound, C1311, exhibited significant cytotoxic activity in vitro and in vivo for a range of colon tumors (both murine and human) and is currently under clinical trials (Denbrok et al.; Hyzy et al.). COXEN might also prove useful for subsetting patients or for “personalizing” their treatment. Currently, the hope is that gene expression profiles obtained from a patient's tumor can be compared with the expression profiles from other tumors of the same organ, grade, and stage to assist in prognosis and selection of therapy. The results described here for COXEN reinforce the idea that it is best to focus on the subset of genes that constitutes a signature of drug sensitivity. Another possibility is the following: If, in the future, a drug has been used, and responses to it recorded, for one type of cancer, its utility in a second type might be predicted by COXEN if both types have been profiled at the molecular level. In other words, the first type of cancer might provide a “training set” with at least some power to predict activity in the second. That strategy would be particularly useful with respect to orphan cancers for which clinical studies are lacking and treatments are empirical. For that type of application, the COXEN discovery algorithm could be limited to drugs that are currently FDA approved for oncological applications.

Generically this approach has even wider application. For example, COXEN is potentially useful whenever one has a combination of drug sensitivity and molecular profile data on one panel of cell types (or on a panel of molecular screens) and wants to use that information to predict chemosensitivity in a panel for which there are only the molecular profile data. For the analyses described here, the essential inputs to the algorithm for each compound were (i) a vector consisting of the compound's pattern of activity against the NCI-60 cell lines; (ii) a matrix consisting of gene expression profiles of the NCI-60. More generally, any matrix of cell characteristics (e.g., protein expression, DNA copy number, occurrence of mutations, etc.) could be substituted; (iii) a matrix consisting of gene expression data for the panel for which sensitivities are to be predicted (e.g., the BLA-40 or the breast sample set). However, the two gene expression sets must include a sufficient number of genes in common. Preferably, they would have been obtained using the same microarray or other platform but, as in the clinical example here, not necessarily so.

DISCUSSION (COMBINATION AGENTS): Herein, we combined a novel mathematical approach (misclassification penalized posterior probabilities) with comprehensive gene expression profiles of 40 urothelial cell lines, to discover high-performance molecular prediction models for single and combination chemotherapeutic sensitivity. The high performance characteristics of the predictive models obtained in this study may be due to several factors. First, we used a panel of cancer cell lines derived from only one histological type, urothelial cancer. In contrast to the NCI60 cancer cell panel, which is comprised of cell lines from multiple anatomic origins, a single anatomic origin should eliminate confounding and biased gene expression signals that represent tissue-dependent sensitivity to different chemotherapy agents. Furthermore, the majority of the cell lines used in this study are derived from invasive or metastatic human urothelial tumors which represent the typical patient population that would receive systemic chemotherapy. Hence, we anticipate that these prediction models may be applicable to clinical urothelial cancer. This conclusion is supported by the observation that cisplatin, a drug used in current clinical treatment of urothelial cancer, was highly effective in our assay (i.e., 16/40 cell lines meeting the chemosensitive criterion).

To identify gene prediction models for chemosensitivity, we used the misclassification-penalized posterior (MiPP) method. Several studies have demonstrated good predictive classification of cancer subtypes and prognosis using methods that require large numbers of (>50) genes while models that are dependent on only a small number of predictive genes has been limited despite the obvious practical advantages. The MiPP method combines the best of both approaches by maintaining excellent predictive accuracy with a small set of genes that are easy to evaluate in human tumors using currently available techniques, such as real time RT-PCR. This feature is a significant advantage as we begin to prospectively evaluate these genes for their ability to predict tumor response in patients treated with drug combinations.

The approach taken here led to the identification of predictive gene models for each of the three drugs. Cisplatin model 1 is comprised of TGM2, MOAP 1, HIST2H2AA, MRPS30; Model 2 contains CAV2, LCP1, and MOAP1 and Model 3 includes CCNG2, PEG10, and WNT5B. By examining the function of the genes encompassed by these models, a common functional theme was noted, that is, their direct (TGM2, MOAP 1, and CAV2) or indirect (H1ST2H2AA, and LCP1) participation in apoptosis. Modulator of apoptosis 2 (MOAP2) is an important component of the pathway that links death receptors and the apoptotic machinery. Caveolin 2 (CAV2) is a major component of the inner surface of caveolae, and is implicated in the control of cellular growth, signal transduction, lipid metabolism, and apoptosis. LCP1 or lymphocyte cytosolic protein1 is found in hemopoietic cell lineages and also in many types of malignant human cells of non-hemopoietic origin. Cyclin G2 (CCNG2) is a member of the Cyclin family. Northern blot analysis revealed that cyclin G2 mRNA fluctuates throughout the cell cycle with peak expression in late S phase. Furthermore, cyclin G2 is induced by the DNA damaging agent actinomycin D.

Models for Paclitaxel included several genes involved in essential eukaryotic cell functions such as protein modification (PLAT), spermatogenesis and cell differentiation (DZIP1) and negative autocrine growth factor regulation (LGALS1). However, perhaps the most interesting of this group is KIF14. This gene is responsible for microtubule motor activity and is expressed at very low levels in normal tissue samples, compared to significantly increased expression in the majority of tumor samples. Its overexpression may lead to rapid mitoses, potentially leading to aneuploidy. KIF14 overexpression is most striking in retinoblastoma, lung, breast, thymus, and tumors and associated with decreased survival in lung cancer. This relationship to paclitaxel sensitivity is intriguing, since this drug promotes the assembly of microtubules from tubulin dimers and stabilizes microtubules by preventing depolymerization, thus inducing abnormal arrays of microtubules throughout the cell cycle.

Thus, we have developed and validated a novel molecular chemosensitivity prediction model for commonly used combinations of cisplatin, paclitaxel, and gemcitabine, using only the results of their individual drug responses. We believe this prediction strategy warrants prospective validation in the clinical setting and, given the parsimonious nature of the predictions shown here, should be straightforward to implement.

Supplementary Materials and Methods

NCI-60 panel and drug potency data The NCI-60 panel consists of 60 cancer cell lines across nine different types of human cancer: breast (6), colon (7), central nerve system (6) leukemia (6), lung (9), melanoma (10), ovarian (6), prostate (2), and renal (8). The in vitro drug screening potency data of NCI-60 provide information-rich pharmacological profiles of the compounds in terms of 60 potency values for each compound. The potency of each drug compound is summarized with several dose concentrations on the 60 cell lines such as GI50 (Growth Inhibition 50), the minimum dose concentration that inhibits the growth of each cell line 50% in comparison with untreated control under the in vitro 48 hr microtiter plate assay used. For this study we used the public NCI-60 drug potency database updated in September 2005, which comprises log(GI50) values on 45,545 compounds, available at the Developmental Therapeutics Programs of the US National Cancer Institute.

NCI-60 gene expression profiling Our protocols for cell culture, cell harvests, and RNA purification, and microarray studies are being described in detail elsewhere (Shankavaram, et al., manuscript in preparation). Briefly, seed cultures of the 60 cell lines were drawn from aliquoted stocks, passaged once in T-162 flasks, and monitored frequently for degree of confluence. The medium was RPMI-1640 with phenol red, 2 mM glutamine, and 5% fetal bovine serum. For compatibility with our other profiling studies, all fetal bovine serum was obtained from the same large batches as were used by DTP for the drug screen. One day before harvest, the cells were re-fed. Attached cells were harvested at ˜80% confluence, as assessed for each flask by phase microscopy. Suspended cells were harvested at ˜0.5×106 cells/mL. In pilot studies, samples of medium showed no appreciable change in pH between re-feeding and harvest, and no color change in the medium was seen in any of the flasks harvested. The time from incubator to stabilization of the preparation was kept to <1 min. Total RNA was purified using the Qiagen (Valencia, Calif.) RNeasy Midi Kit according to manufacturer's instructions. The RNA was then quantitated spectrophotometrically and aliquoted for storage at −80° C. The samples were labeled and hybridized to HG-U133A GeneChip® microarrays according to standard procedures by GeneLogic, Inc., which can be obtained at the NCI website (http://discover.nci.nih.gov/).

BLA-40 gene expression profiling Applicants recently collected 40 commonly used human bladder cancer cell lines 20, here designated the “BLA-40 cell panel.” Gene expression profiling for the BLA-40 was also carried out using HG-U133A arrays on duplicate samples generated from independent cell cultures as described 20. When the image files of the NCI-60 and BLA-40 cell lines passed quality-control checks, they were analyzed using the RMA analysis software for GeneChip® data to obtain expression levels.

Identification of gene co-expression extrapolation signatures (FIG. 2A). Starting with the set of candidate chemosensitivity genes for a given compound, we next identified a subset of those genes that showed concordant co-expression relationships between the NCI-60 and BLA-40 cancer cell line panels. To parameterize such relationships, we calculated co-expression extrapolation coefficient (CEEC), rc(j), for gene j in the following way: Using the gene expression data, we constructed two correlation matrices (of dimension n×n) for the set of n candidate chemosensitivity genes. The two correlation matrices, one for the NCI-60, the other for the BLA-40, were evaluated as U=[Uij]n×n and V=[Vij]n×n, where Uij and Vij are the correlation coefficients between genes i and j in the NCI-60 and BLA-40, respectively. Then, rc(j) is defined as:

$$rc ( j ) = \frac{{\sum }_{k = 1}^{n} ( {U}_{kj} - {\overline{U}}_{k} ) ( {V}_{kj} - {\overline{V}}_{k} )}{\sqrt{{\sum }_{k = 1}^{n} {( {U}_{kj} - {\overline{U}}_{k} )}^{2}} \sqrt{{\sum }_{k = 1}^{n} {( {V}_{kj} - {\overline{V}}_{k} )}^{2}}}$$

where Ūₖ and V̅ₖ are the mean correlation coefficients of the row-k correlation coefficient vectors for the NCI-60 and BLA-40. We used rc as a parameter that reflects the degree of co-expression extrapolation of gene k with the set of n genes between the NCI-60 and BLA-40 cell lines. If rc(j) exceeded a cut-off criterion (e.g., 98th percentile of the corresponding random distribution generated by randomly shuffling the gene identities between the two sets), gene j was selected as a gene for co-expression extrapolation between the two panels. Since gene j was selected from the set of n candidate chemosensitivity predictors, it had that pharmacological characteristic as well.

Misclassification-Penalized Posterior classification for chemosensitivity prediction The CEEC probes (e.g. Table S1A) were then used to develop chemosensitivity prediction models by searching for the most parsimonious prediction models that best classified NCI-60 cell lines as sensitive or resistant to the drug (e.g., cisplatin). For that purpose, we used the Misclassification-Penalized Posterior (MiPP) classification algorithm, which we have described previously and briefly summarized here. In brief, MiPP is based on stepwise incremental classification modeling and double cross-validation of model performance. The first cross-validation is based on random splitting of the whole data set into a training set and an independent test set for external model validation; the second is an n-fold cross-validation on the training set in order to avoid the pitfalls of a large-screening search and to obtain the most parsimonious optimal prediction model(s). Multiple independent splits of the training and test set combinations are generated. Those independent splits result in multiple prediction models. The multiple models are then re-evaluated using a large number (e.g., 100) of random splits of test and training sets to obtain their objective prediction accuracy confidence bounds. From that confidence interval evaluation on the prediction performance, together with mean misclassification error rates (ER), were obtained for each of the candidate prediction models. The final prediction of a cell line as “sensitive” or “resistant” was based on the cell's (posterior) classification probability of being sensitive from (3-5) top prediction models based on these confidence bounds away from 0.5, i.e. random coin tossing. It turns out that MiPP is particularly useful in our COXEN algorithm since it searches for the most parsimonious gene prediction models, especially based on the small number of co-expression extrapolated genes between the NCI-60 and each of target validation sets by efficiently utilizing non-redundant predictive information from the candidate modeling genes. The open-source MiPP package in R is available at the Bioconductor website (www.bioconductor.org). See the original studies for technical details.

Hierarchical clustering based on CEEC signatures To examine the overall expression patterns of the CEEC genes, we used those genes to co-cluster 22 the combined microarray data of the NCI-60 and the BLA-40 cells, or breast cancer patients, that were sensitive and resistant, or responsive and non-responsive, to each treated compound. As shown in FIG. 2C for cisplatin between the NCI-60 and the BLA-40, the cells clustered largely according to their sensitivity or resistance, not according to their organ of origin or whether they were from the NCI-60 or BLA-40 panel. That visual result strongly indicates that the genes picked out to form the CEEC signature are better markers for response to cisplatin than they are to the other variables, such as histological subtype for example. In stark contrast, the NCI-60 and BLA-40 cell types separate almost completely, irrespective of cisplatin response, when they were hierarchically clustered on the basis of gene profiles not selected with relation to drug sensitivity. This is shown in FIG. 2B where clustering was performed on the basis of the top 50 differentially expressed genes. Results similar to those in FIGS. 2B and C were obtained for paclitaxel on the BLA-40 cell lines (FIG. 2D-E) and the docetaxel (DOC-24) and tamoxifen (TAM-60) clinical trials (data not shown).

Discovery of novel candidate anticancer compounds from the NCI-60 screening data We applied COXEN in a novel drug discovery capacity for human bladder cancer since we would need to evaluate a hit to validate any findings. Using the BLA-40 panel for such screening, we repeated all the steps shown above by 1) identifying differentially expressed probes between each drug's sensitive and resistant cell lines of NCI-60 for the entire 45,545 anticancer compounds available in the NCI-60 public drug database (updated in September 2005), 2) discovering co-expression extrapolated signatures between NCI-60 and BLA-40 panels for every one of these compounds, 3) developing MiPP prediction models of each compound on the NCI-60, and 4) predicting in silico chemosensitivity of the BLA-40 panel for each of these compounds (FIG. 4A). For this large-screening discovery we developed an automated computing program in order to screen the candidate compounds efficiently. This computational automation required some additional steps: 1) evaluation of drug potency by examining each drug's (ordered) log(GI50) values and 2) calculation of average drug response rates on the BLA-40 cell lines from the top five identified MiPP models. For this intensive computation, a cluster computer with customized parallel programming was used for 54 days (24 hrs/day) on a 32-node cluster computer, with each node comprised of an Xserve G5 2 GHz CPUs with 8 GB memory on Mac OS X 10.3.8 at the University of Virginia. Those selected were further ranked by the predicted proportions of sensitive cell lines in the MiPP chemosensitivity prediction models.

Supplementary Tables

TABLE S1
Co-expression extrapolation signature probes for
chemosensitivity prediction of cisplatin and paclitaxel between NCI-60 and BLA-40
panels. 18 probes for cisplatin and 13 for paclitaxel identified as a function of significant
differential expression between NCI-60 sensitive and resistant cell lines and with their high
co-expression extrapolation coefficients between NCI-60 and BLA-40 cell line panels.

| Affymetrix | Gene | Locus | Gene acc. | |
|---|---|---|---|---|
| ID | symbol | ID | number | Description |
| Cisplatin | | | | |
| 200606_at | DSP | 1832 | NM_004415 | Desmoplakin |
| 201428_at | CLDN4 | 1364 | NM_001305 | claudin 4 |
| 201839_sat | TACSTD1 | 4072 | NM_002354 | tumor-associated calcium signal |
| | | | | transducer 1 |
| 203287_at | LAD1 | 3898 | NM_005558 | ladinin 1 |
| 203407_at | PPL | 5493 | NM_002705 | Periplakin |
| 203713_s_at | LLGL2 | 3993 | NM_004524 | lethal giant larvae homolog 2 |
| | | | | (Drosophila) |
| 205709_s_at | CDS1 | 1040 | NM_001263 | CDP-diacylglycerol synthase 1 |
| 206722_s_at | EDG4 | 9170 | NM_004720 | lysophosphatidic acid G-protein- |
| | | | | coupled receptor, 4 |
| 209873_s_at | PKP3 | 11187 | AF053719 | Plakophilin 3 |
| 210058_at | MAPK13 | 5603 | BC000433 | mitogen-activated protein kinase 13 |
| 210059_s_at | MAPK13 | 5603 | BC000433 | mitogen-activated protein kinase 13 |
| 210480_s_at | MYO6 | 4646 | U90236 | myosin VI |
| 210761_s_at | GRB7 | 2886 | AB008790 | growth factor receptor-bound protein 7 |
| 218780_at | HOOK2 | 29911 | NM_013312 | hook homolog 2 (Drosophila) |
| 218966_at | MYO5C | 55930 | NM_018728 | myosin VC |
| 219395_at | RBM35B | 80004 | NM_024939 | RNA binding motif protein 35A |
| 219513_s_at | SH2D3A | 10045 | NM_005490 | SH2 domain containing 3A |
| 31846_at | RHOD | 29984 | AW003733 | ras homolog gene family, member D |
| Paclitaxel | | | | |
| 201478_s_at | DKC1 | 1736 | U59151 | dyskeratosis congenita 1, dyskerin |
| 201479_at | DKC1 | 1736 | NM_001363 | dyskeratosis congenita 1, dyskerin |
| 203221_at | TLE1 | 7088 | AI758763 | Transducin-like enhancer of split 1 |
| 203625_xat | SKP2 | 6502 | BG105365 | S-phase kinase-associated protein 2 |
| | | | | (p45) |
| 203895_at | PLCB4 | 5332 | AL535113 | phospholipase C, beta 4 |
| 203896_s_at | PLCB4 | 5332 | NM_000933 | phospholipase C, beta 4 |
| 204767_s_at | FEN1 | 2237 | BC000323 | flap structure-specific endonuclease 1 |
| 204768_s_at | FEN1 | 2237 | NM_004111 | flap structure-specific endonuclease 1 |
| 209654_at | KIAA0947 | 23379 | BC004902 | NA |
| 211651_s_at | LAMB1 | 3912 | M20206 | laminin, beta 1 |
| 213918_s_at | NIPBL | 25836 | BF221673 | Nipped-B homolog (Drosophila) |
| 218979_at | C9orf76 | 80010 | NM_024945 | chromosome 9 open reading frame |
| | | | | 76 |
| 219000_s_at | DCC1 | 79075 | NM_024094 | NA |

Table S2. Co-expression extrapolation signature probes for chemosensitivity prediction of paclitaxel and tamoxifen between the NCI-60 panel and breast cancer tissues. Probes identified as a function of significant differential expression between NCI-60 responder and nonresponder cell lines, and then with their high co-expression extrapolation coefficients between NCI-60 and each of the two patient populations from the docetaxel (14 probes) and tamoxifen (8 probes) breast cancer clinical trials.

TABLE S2
Co-expression extrapolation signature probes for
chemosensitivity prediction of paclitaxel and tamoxifen between the NCI-60 panel and
breast cancer tissues. Probes identified as a function of significant differential expression
between NCI-60 responder and nonresponder cell lines, and then with their high co-
expression extrapolation coefficients between NCI-60 and each of the two patient
populations from the docetaxel (14 probes) and tamoxifen (8 probes) breast cancer clinical trails.

| Affymetrix | Gene | Locus | Gene acc. | |
|---|---|---|---|---|
| ID | symbol | ID | Number | Description |
| Paclitaxel* | | | | |
| 211915_s_at | TUBB4Q | 56604 | U83110 | tubulin, beta polypeptide 4, member Q |
| 216022_at | WNK1 | 65125 | AL049278 | WNK lysine deficient protein kinase 1 |
| 208387_s_at | MMP24 | 10893 | NM_006690 | matrix metallopeptidase 24 (membrane- |
| | | | | inserted) |
| 202312_s_at | COL1A1 | 1277 | NM_000088 | collagen, type I, alpha 1 |
| 210738_s_at | SLC4A4 | 8671 | AF011390 | solute carrier family 4 |
| 214133_at | MUC6 | 4588 | AI611214 | mucin 6, gastric |
| 209995_s_at | TCL1A | 8115 | BC003574 | T-cell leukemia/lymphoma 1A |
| 214589_at | FGF12 | 2257 | AL119322 | fibroblast growth factor 12 |
| 209552_at | PAX8 | 7849 | BC001060 | paired box gene 8 |
| 204505_s_at | EPB49 | 2039 | NM_001978 | erythrocyte membrane protein band 4.9 |
| | | | | (dematin) |
| 212974_at | DENND3 | 22898 | AI808958 | DENN/MADD domain containing 3 |
| 215904_at | MLLT4 | 4301 | AL049698 | myeloid/lymphoid or mixed-lineage |
| | | | | leukemia |
| 213560_at | GADD45B | 4616 | AV658684 | growth arrest and DNA-damage-inducible, |
| | | | | beta |
| 211886_s_at | TBX5 | 6910 | U80987 | T-box 5 |
| Tamoxifen | | | | |
| 200970_s_at | SERP1 | 27230 | AL136807 | NA |
| 201632_at | EIF2B1 | 1967 | NM_001414 | eukaryotic translation initiation factor 2B, |
| | | | | subunit 1 alpha |
| 204326_x_at | MT1L | 4500 | NM_002450 | metallothionein 1L |
| 206664_at | SI | 6476 | NM_001041 | sucrase-isomaltase (alpha-glucosidase) |
| 208581_x_at | MT1X | 4501 | NM_005952 | metallothionein 1X |
| 208869_s_at | GABARAPL1 | 23710 | AF087847 | GABA(A) receptor-associated protein like 1 |
| 210907_s_at | PDCD10 | 11235 | BC002506 | programmed cell death 10 |
| 212730_at | DMN | 23336 | AK026420 | desmuslin |

Rationale for using paclitaxel instead of docetaxel is explained in the text

The disclosures of each and every patent, patent application, and publication cited herein are hereby incorporated by reference herein in their entirety.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Accordingly, the present invention is not intended to be limited to the embodiments shown herein but is to be accorded the widest scope consistent with the principles and novel features disclosed herein.

What is claimed is:

1. A method for predicting the activity of at least one agent, comprising:
(a) determining an agent's pattern of activity against a 1^{st} cell set (CS-1), wherein this activity determination shows which cells are sensitive and resistant to the agent;
(b) measuring a set of molecular characteristics (MC-1) for each cell represented in CS-1;
(c) selecting a subset of molecular characteristics (MC-2) from MC-1 for each cell represented in CS-1, each subset comprising: those molecular characteristics that most accurately predict the agent's activity against each cell represented in CS-1;
(d) measuring the same set of molecular characteristics (MC-3) as MC-1 for each cell represented in a 2^{nd} cell set (CS-2), wherein CS-2 contains cells that differ from those of CS-1;
(e) identifying a set of molecular characteristics (MC-4) that is a subset of MC-2 and MC-3, wherein MC-4, comprises: a set of molecular characteristics concordant to sets MC-2 and MC-3; and,
(f) predicting the agent's activity against each cell represented in CS-2, comprising: using a multivariate classification algorithm that compares the agent's determined activity against CS-1 with MC-4.

2. The method of claim 1, wherein step (f), comprises:
(f-i) prior to predicting the agent's activity against CS-2, using a multivariate algorithm to reduce the number of molecular characteristics of MC-4 to form MC-4A, comprising: evaluating different combinations and selecting the best combinations of the molecular characteristics in MC-4 with a multivariate classification algorithm for their overall prediction performance of the agent's activity against CS-1, or alternatively, combining the information in MC-4 with a multivariate dimension reduction algorithm to form MC-4A; and,
(f-ii) predicting the agent's activity against each cell represented in CS-2, comprising: using a multivariate classification algorithm that compares the agent's determined activity against CS-1 with MC-4A.

3. The method of claim 2, wherein the activity against CS-2 is estimated by observing how closely the molecular characteristics MC-4A of each cell in CS-2 match, in terms of the presence and expression levels of the same characteristics, the molecular characteristics MC-4A of the sensitive and resistant cells in CS-1.

4. The method of claim 1, wherein the method further comprises: replacing (f) with at least the following:
(g) measuring a set of molecular characteristics (MC-5) for each cell represented in a 3 cell set (CS-3), wherein CS-3 contains cells that differ from those of CS-1 and CS-2; and;
(h) identifying a set of molecular characteristics (MC-6) that is a subset of MC-2 and MC-5, wherein MC-6, comprises: a set of molecular characteristics concordant to sets MC-2 and MC-5;
(i) identifying a set of molecular characteristics (MC-7) that is a subset of concordant sets MC-4 and MC-6, wherein MC-7, comprises: a set of molecular characteristics common to sets MC-4 and MC-6;
(j) predicting the agent's activity against each cell represented in CS-2 and CS-3, comprising: using a multivariate classification algorithm that compares the agent's determined activity against CS-1 with MC-7.

5. The method of claim 4, wherein step (j), comprises:
(j-i) prior to predicting the agent's activity against CS-2 and CS-3, using a multivariate algorithm to reduce the number of molecular characteristics of MC-7 to form MC-7A, comprising: evaluating different combinations and selecting the best combinations of the molecular characteristics in MC-7 with a multivariate classification algorithm for their overall prediction performance of the agent's activity against CS-1, or alternatively, combining the information in MC-7 with a multivariate dimension reduction algorithm to form MC-7A; and,
(j-ii) predicting the agent's activity against each cell represented in CS-2 and CS-3, comprising: using a multivariate prediction algorithm that compares the agent's determined activity against CS-1 with MC-7A.

6. The method of claim 4, wherein the agent is from NCI-60 anticancer drug screening database.

7. The method of claim 5, wherein the activity against CS-2 and CS-3 is estimated by observing how closely the molecular characteristics MC-7A of each cell in CS-2 and CS-3 match, in terms of the presence and expression levels of the same characteristics, those of sensitive and resistant cells in CS-1.

8. The method of claim 1, wherein the activity determined is the agent's cytostaticability (growth inhibition) and/or cytotoxicity (cell death) against each cell type in CS-1.

9. The method of claim 1, wherein each cell set is a cancer cell set and the activity being tested is anti-cancer activity.

10. The method of claim 1, wherein CS-1 is a panel of cancer cells.

11. The method of claim 10, wherein the panel of cancer cells is the NCI-60 panel.

12. The method of claim 1, wherein CS-2 is a set of cells derived from human laboratory cell lines.

13. The method of claim 12, wherein the human laboratory cell lines are cancer cell or endothelial cell lines.

14. The method of claim 4, wherein CS-3 is a set of cells derived from human tissue samples.

15. The method of claim 12, wherein CS-3 is a set of cancer cells derived from human tissue samples of the same type of cancer as that of CS-2.

16. The method of claim 1, wherein the molecular characteristics are selected from (i) profiling of gene expression, (ii) profiling of SNPs (single nucleotide polymorphisms), (iii) profiling of protein expression.

17. The method of claim 16, wherein the molecular characteristics are mRNA expression profiles.

18. A method for selecting a patient-specific API, comprising:
(a) determining each API's pattern of activity against a 1^{st} cell set (CS-1), wherein this activity determination shows which cells are sensitive and resistant to the API;
(b) measuring a set of molecular characteristics (MC-1) for each cell represented in CS-1;
(c) selecting a subset of molecular characteristics (MC-2) from MC-1 for each cell represented in CS-1, each subset comprising: those molecular characteristics that most accurately predict the API's activity against each cell represented in CS-1;
(d) measuring a set of molecular characteristics (MC-3) for a patient's tissue sample (TS-1), wherein the patient is in need of therapy;
(e) identifying a set of molecular characteristics (MC-4) that is a subset of MC-2 and MC-3, wherein MC-4, comprises: a set of molecular characteristics concordant to sets MC-2 and MC-3;
(f) using a multivariate classification algorithm to reduce the number of molecular characteristics of MC-4 to form MC-4A, comprising: evaluating different combinations and selecting the best combinations of the molecular characteristics in MC-4 with a multivariate classification algorithm for their overall prediction performance of the API's activity against CS-1, or alternatively, combining the information in MC-4 with a multivariate dimension reduction algorithm to form MC-4A; and,
(g) creating prediction models, comprising: using a multivariate classification algorithm to predict each API's activity against CS-1 with MC-4A;
(h) predicting each API's activity against TS-1 using MC-4A in the prediction models.

19. The method of claim 18, wherein the activity against TS-1 is estimated by observing how closely the molecular characteristics MC-4A of each cell in TS-1 match, in terms of the presence and expression levels of the same characteristics, those of sensitive and resistant cells in CS-1.

20. The method of claim 18, wherein CS-1 corresponds to the set of NCI-60 cancer cell lines or a similar set of cancer cell line panels.

21. The method of claim 18, wherein CS-1 corresponds to a set of patients and the data for (a) and (b) are collected from the response data and patient microarray data of the patients.

22. The method of claim 21, wherein the patient response data and microarray data are from patients who have received therapy for a cancer or other disease.

23. The method of claim 18, further comprising:
(i) repeating steps (a)-(h) for a group of APIs resulting in a data set of each API's activity against TS-1 as well as a sensitivity and resistance characteristics against CS-1;
(j) selecting first set of combinations of at least 2 APIs by comparing their predicted activities against TS-1 with their known molecular mechanisms and toxicities to arrive at highly active combinations whose expected toxicity levels are tolerable to the patient;
(k) selecting a second set of combinations, wherein the second set if a subset of the first set of combinations, the second set being selected by choosing those combinations whose individual API sensitivity and resistance characteristics are the least correlated;
(l) predicting the combined activities of the second set of combinations of APIs in two ways, (I) assuming those APIs' activities are independent or (II) assuming their activities are correlatively additive on the basis of the sensitive and resistance characteristics on CS-1.

24. A method of treating cancer, comprising: administering a therapeutically effective amount of a compound of Table 3, 4, 5, 6, or 7 or a pharmaceutically acceptable salt thereof, wherein the cancer is selected from breast, bladder, prostate, melanoma, and pancreatic.
